(19) United States
(12) Patent Application Publication
(10) Pub. No.: US 2024/0053337 A1
(43) Pub. Date: Feb. 15, 2024

(54) COMPOSITIONS AND METHODS FOR SINGLE CELL ANALYTE DETECTION AND ANALYSIS

(71) Applicant: 10x Genomics, Inc., Pleasanton, CA (US)

(72) Inventor: Wyatt James MCDONNELL, Pleasanton, CA (US)

(21) Appl. No.: 18/486,264

(22) Filed: Oct. 13, 2023

Related U.S. Application Data
(63) Continuation of application No. PCT/US2022/024652, filed on Apr. 13, 2022.
(60) Provisional application No. 63/174,940, filed on Apr. 14, 2021.
(60) Provisional application No. 63/254,019, filed on Oct. 8, 2021.

(51) Int. Cl. G01N 33/569; G01N 33/53; G01N 33/68

(52) U.S. Cl. CPC G01N 33/56972; G01N 33/5308; G01N 33/6863; G01N 2458/10

(57) ABSTRACT

The present disclosure generally relates to compositions and methods for secreted analyte detection and analysis. The analytes detected and analyzed are mapped to a cell or nucleus having secreted the analyte and are useful to characterize the cell or nucleus. The ability to understand analyte secretion events has implications for the improvement of drug delivery, cell profiling, and diagnostic development.

### CROSS-REFERENCE TO RELATED APPLICATIONS

This application claims benefit of priority to U.S. Provisional Application Ser. No. 63/174,940 filed Apr. 14, 2021, and U.S. Provisional Application Ser. No. 63/254,019 filed Oct. 8, 2021, both of which are herein incorporated by reference in their entireties.

### BACKGROUND

The advent of reporter oligonucleotides in microfluidic systems has enabled the profiling of hundreds of cellular antigens. The profiling of cellular antigens can be combined with assays that detect and quantify other cellular information such as transcriptome/gene expression, immune receptor sequencing, chromatin accessibility and other features.

To profile cellular antigens, antibodies with attached reporter oligonucleotides are typically used as reagents. The antibodies bind their cellular antigens and the attached reporter oligonucleotides are captured and utilized in downstream reactions for antigen detection.

There is a need to improve these existing reagents and methods that will reduce their cost, increase their sensitivity and add to their functionality. Such improvements have implications for drug delivery, drug development and cell profiling.

### SUMMARY

Provided herein are, inter alia, methods and compositions useful for identifying or characterizing a cell or a nucleus as secreting an analyte, characterizing a cell or a nucleus as having an analyte secretion profile, identifying a cell or a nucleus as a cell or a nucleus of interest, or enriching a cell or a nucleus as a cell or a nucleus of interest.

In an aspect, the description provides for a method of identifying or characterizing a cell or a nucleus as secreting an analyte. In the method, a partition is provided. The partition includes a cell or a nucleus, a plurality of nucleic acid barcode molecules comprising a partition-specific barcode sequence and a reporter agent for detecting the analyte. The reporter agent includes a first aptamer that comprises: a first domain that binds the analyte; a second domain that includes a reporter oligonucleotide; and a third domain which is responsive to the analyte binding the first domain. A barcoded nucleic acid molecule comprising a sequence of the reporter oligonucleotide or reverse complement thereof and the partition-specific barcode sequence or reverse complement thereof is generated. The barcoded nucleic acid molecule may be generated in the partition.

In another aspect, the description provides for a method of identifying or characterizing a cell or a nucleus as secreting an analyte. In the method, a partition is provided. The partition includes a cell or a nucleus, a detectable moiety and a reporter agent for detecting the analyte. The reporter agent includes: (i) a first domain that binds the analyte; and (ii) a second domain responsive to the analyte binding the first domain. The second domain includes a subdomain capable of uptake of the detectable moiety in response to the analyte binding to the first domain. The uptake of the detectable moiety by the subdomain is detected.

In yet another aspect, the disclosure provides for a method of identifying or characterizing a cell or a nucleus as secreting an analyte. In the method, a partition is provided. The partition includes a cell or a nucleus, a detectable moiety, and a reporter agent for detecting the analyte. The reporter agent includes a first aptamer. The first aptamer has a first domain that binds to the analyte and a second domain responsive to the analyte binding the first domain. The second domain has a subdomain capable of release of the detectable moiety in response to the analyte binding the first domain. Release of the detectable moiety by the subdomain is detected.

In yet a further aspect, the disclosure provides a method of identifying or characterizing a cell or a nucleus as secreting an analyte. In the method, a partition is provided. The partition includes a cell or a nucleus, a plurality of nucleic acid barcode molecules that include a partition-specific barcode sequence, a first aptamer and a second aptamer. The first aptamer includes a first reporter oligonucleotide, a first linking sequence (e.g., a first ligation handle sequence) and a lipophilic moiety that embeds the first aptamer in the cell or nuclear membrane. The second aptamer includes a second reporter oligonucleotide, a second linking sequence (e.g., ligation handle sequence) and a domain that binds the analyte. The first reporter oligonucleotide has a first reporter barcode sequence specific to the first aptamer and a first capture sequence. The second reporter oligonucleotide has a second reporter barcode sequence specific to the second aptamer and a second capture handle sequence. The first and the second capture handle sequences are configured to couple to a capture sequence comprised in a first of the plurality of nucleic acid barcode molecules. In the method, the partition is subject to conditions sufficient (i) to embed the lipophilic moiety in the cell membrane of the cell or nuclear membrane of the nucleus, (ii) to link the first aptamer and second aptamer via the first and second linking sequences, and (iii) to bind a secreted analyte from the cell or the nucleus via the domain of the second aptamer.

In yet a further aspect, the disclosure provides a method of identifying or characterizing a cell or a nucleus as secreting an analyte. In the method, a reaction mixture, or a portion thereof, is partitioned into a plurality of partitions. The reaction mixture includes a plurality of cells or nuclei, and a reporter agent that includes first and second aptamers. The first aptamer of the reporter agent includes a first reporter oligonucleotide, a first linking sequence (e.g., a first ligation handle sequence) and a lipophilic moiety that embeds the first aptamer in the cell or nuclear membrane. The second aptamer of the reporter agent includes a second reporter oligonucleotide, a second linking sequence (e.g., ligation handle sequence) and a domain that binds the analyte. The first and second linking sequences link the first and the second aptamers. The first reporter oligonucleotide has a first reporter barcode sequence specific to the first aptamer and a first capture sequence. The second reporter oligonucleotide has a second reporter barcode sequence specific to the second aptamer and a second capture handle sequence. The reaction mixture includes a cell or nucleus of the plurality of cells or nuclei coupled to the reporter agent bound to the analyte as a result of (i) the lipophilic moiety of the first aptamer having embedded in the membrane of the cell or nucleus and (ii) the secreted analyte having bound to the domain that binds the analyte of the second aptamer. The partitioning provides a partition of the plurality of partitions that includes (i) the cell or nucleus coupled to the reporter agent bound to the analyte and (ii) a plurality of nucleic barcde molecules that have a partition-specific barcode sequence. Barcoded nucleic acid molecules are generated. The barcoded nucleic acid molecules have a sequence of: (i) the first reporter oligonucleotide or a reverse complement thereof and the partition-specific barcode sequence or a reverse complement thereof and/or (ii) the second reporter oligonucleotide or a reverse complement thereof and the partition-specific barcode sequence or a reverse complement thereof.

In a further aspect, the disclosure provides a partition. The partition includes a cell or a nucleus and a reporter agent. The reporter agent includes a first aptamer. The first aptamer includes a first domain that binds an analyte and a second domain responsive to the analyte binding the first domain.

In another aspect, the disclosure provides a kit. The kit includes instructions for use. The kit also includes a reporter agent that includes a first aptamer. The first aptamer includes a first domain that binds an analyte and second domain response to the analyte binding to the first domain. The kit is for detecting secretion of the analyte by a cell or a nucleus, or characterizing an analyte secretion profile of a cell or a nucleus, or identifying a cell or a nucleus as a cell or a nucleus of interest, or enriching for a cell or a nucleus as a cell or a nucleus of interest.

The foregoing is merely a summary and is illustrative only. It is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 shows an example of a reporter agent comprising a first aptamer, the first aptamer including: a domain comprising reporter oligonucleotide (110); a domain capable binding a secreted analyte (115); and a domain responsive to the analyte binding to the domain capable of binding the secreted analyte (120) which includes a subdomain (105) capable of uptake or release of a detectable moiety (106).

FIG. 2 shows an example of a reporter agent comprising a first aptamer (0201) and a second aptamer (0200) coupled (or tethered) to a cell (250). The first aptamer (0201) includes a domain comprising reporter oligonucleotide (0211); a domain capable binding a secreted analyte (0216); and a domain responsive to the analyte binding to the domain capable of binding the secreted analyte (0221) which includes a subdomain (0206) capable of uptake or release of a detectable moiety. The second aptamer (0200) includes a domain comprising reporter oligonucleotide (0210) and is conjugated to lipophilic moiety (0220). The first and the second aptamers are linked (e.g., ligated) at their respective linking sequences (240) (e.g., their respective ligation handle sequences).

FIG. 3 shows an example of culturing cells (300) that are secreting analytes (310).

FIG. 4 is a schematic example of a first reporter agent (410) binding an analyte (420) secreted by a cell (430).

FIG. 5 shows an example of a cell (505) coupled to a reporter agent that includes a first (550) and second (560) aptamers. The reporter agent's first aptamer (550) is shown binding to a secreted analyte (520) and as being coupled to the second aptamer (530) via linked or ligated first and second linking sequences (540) (e.g., ligation handle sequences). The reporter agent's second aptamer (530) is shown as being coupled to the surface of the cell (505) secreting the analyte (520), e.g., by a lipophilic moiety and/or separate binding site capable of binding a protein on the cell's surface (560).

FIG. 6 provides an example workflow that may utilize a reporter agent comprising an aptamer for detection and analysis of a cell secreted analyte.

FIG. 7 provides exemplary workflows that utilize a reporter agent comprising a first aptamer comprising a domain capable or uptake or release of a detectable moiety in response to binding an analyte and a domain comprising a reporter oligonucleotide in a method for detecting and analyzing analyte secretion by a cell.

FIG. 8A shows an example of a cell (801) forming a cell bead (804).

FIG. 8B shows an example of culturing cells (801) within cell beads (804) to allow or induce secretion of analytes (805).

FIG. 8C shows an example of a reporter agent comprising a first aptamer specific for an analyte (809) and the first aptamer specific for an analyte while bound to the analyte (811) and coupled to a polymer backbone (804).

FIG. 8D shows an example of a hydrogel matrix (824; e.g., a bead) coupled to a reporter agent comprising a first aptamer (814), in which the first aptamer comprises a domain comprising a reporter oligonucleotide (815), and comprises a domain bound to a secreted analyte (825; e.g., cytokine).

FIG. 9 shows an example of a partitioned cell (902) and partitioned cell bead (904).

FIG. 10A shows an example of a barcoded bead that may be used in a partition to couple to a barcode (1002; e.g., a partition-specific barcode) to one or more analytes (e.g., secreted analytes such as cytokines (1006), mRNAs (1004), etc.) of a single cell, thereby associating said one or more analytes with the single cell.

FIG. 10B shows an illustration of the conversion of barcoded analytes into sequencing libraries.

FIG. 11 shows an example of possible simultaneous measurements of secreted analytes, mRNAs and surface proteins of a cell.

FIG. 12 shows an exemplary microfluidic channel structure for partitioning individual biological particles in accordance with some embodiments of the disclosure.

FIG. 13 shows an exemplary microfluidic channel structure for the controlled partitioning of beads into discrete droplets.

FIG. 14 shows an exemplary microfluidic channel structure for delivering barcode carrying beads to droplets.

FIG. 15 shows an exemplary barcode carrying bead.

FIG. 16 illustrates another example of a barcode carrying bead.

FIG. 17 schematically illustrates an example microwell array.

FIG. 18 schematically illustrates an example workflow for processing nucleic acid molecules.

FIG. 19 schematically illustrates examples of labelling agents.

FIG. 20 depicts an example of a barcode carrying bead.

FIGS. 21A, 21B, 21C and 21D schematically depict an example workflow for processing nucleic acid molecules.

FIG. 22 depicts a block diagram illustrating an example of a computing system, in accordance with some example embodiments.

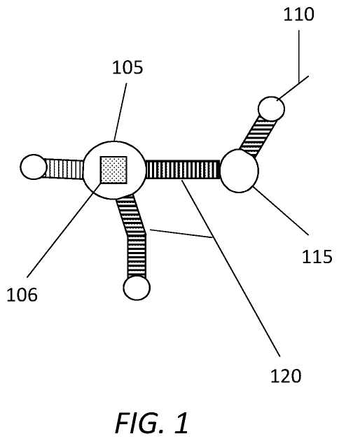

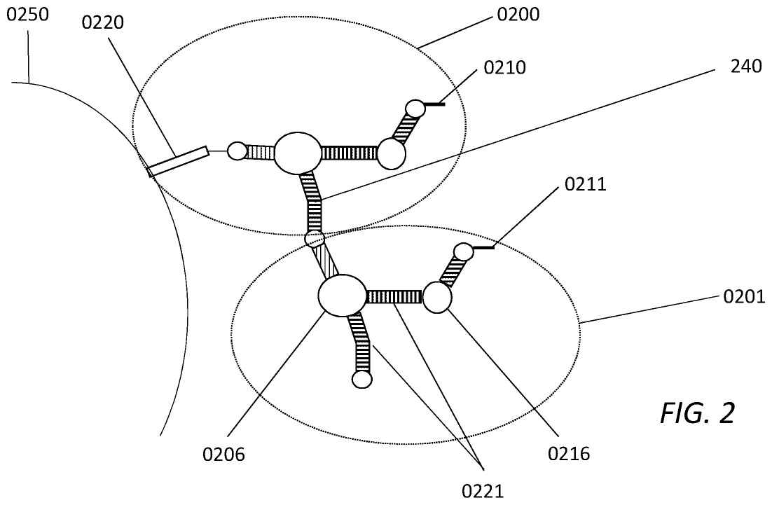

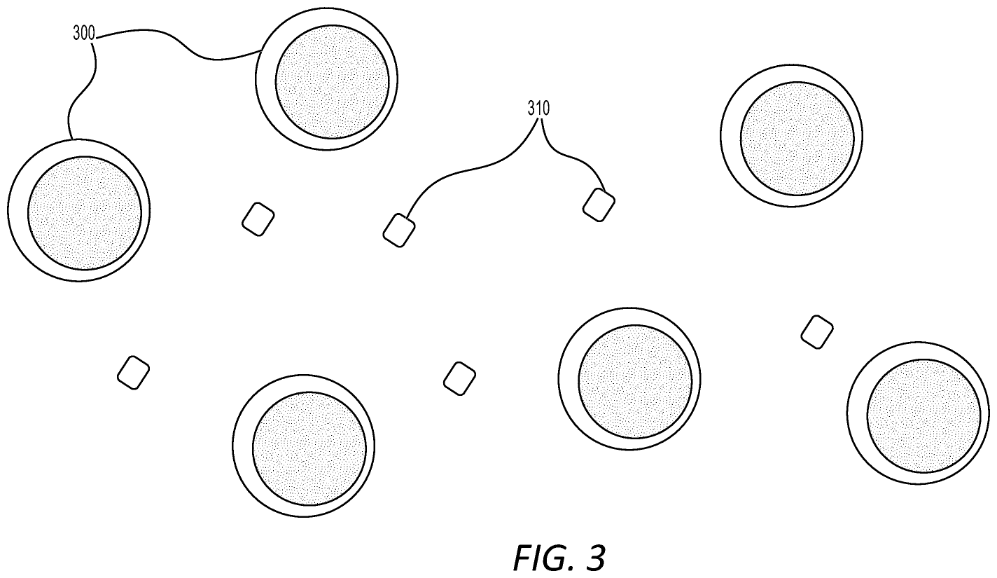

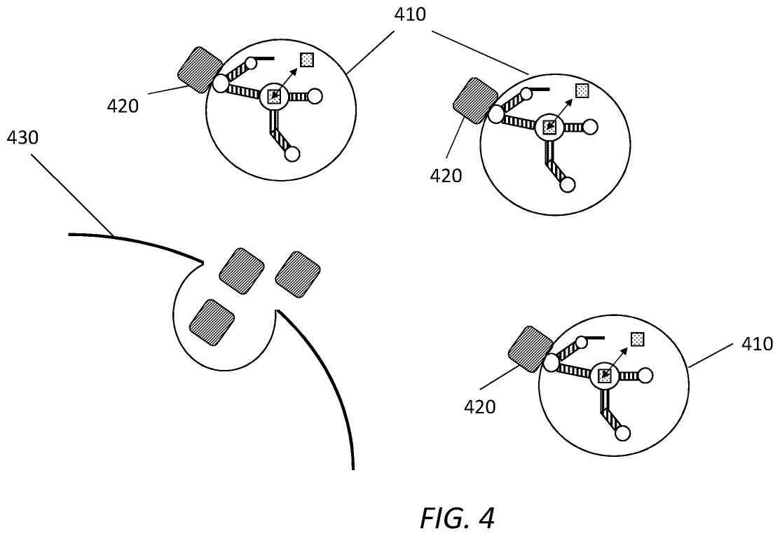

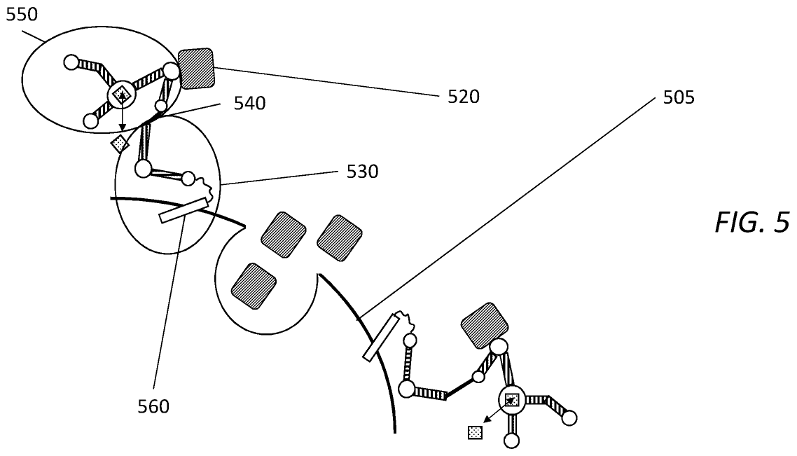

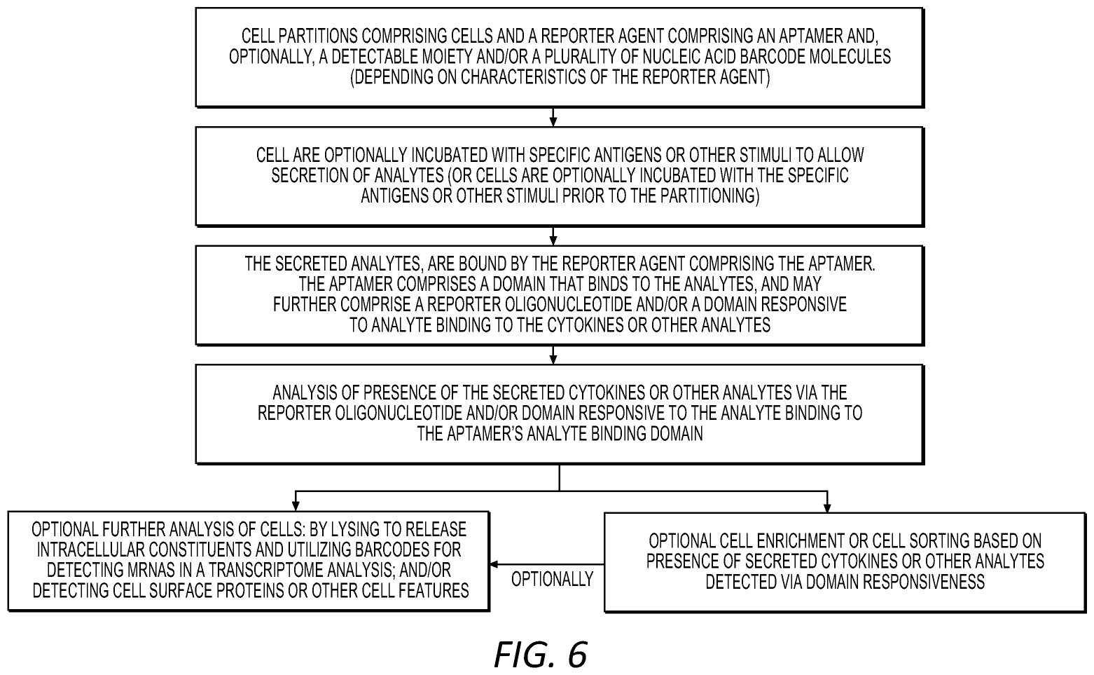

### DETAILED DESCRIPTION OF THE DISCLOSURE

The present disclosure generally relates to, inter alia, compositions, methods, partitions and kit for detecting secretion of an analyte by a cell or a nucleus, or characterizing an analyte secretion profile of a cell or a nucleus, characterizing or identifying a cell or a nucleus as secreting an analyte or identifying a cell or a nucleus as a cell or a nucleus of interest, or enriching for a cell or nucleus as a cell or nucleus of interest. In some embodiments, the compositions, methods, partitions and kits are for detecting secretion of an antibody, or an antigen-binding fragment thereof, by an antibody-secreting cell. Such methods may further characterize the secreted antibodies, or antigen-binding fragments thereof.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B. V., the disclosures of which are incorporated herein by reference

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, a “subject” or an “individual” includes animals, such as human (e.g., human individuals) and non-human animals. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, reptiles, etc. Animals may include, but are not limited to, farm animals, sport animals, and pets. A subject can be a healthy individual, an asymptomatic individual, an individual that has or is suspected of having a disease (e.g., cancer or infection), an individual having a pre-disposition to a disease, an individual that is in need of therapy for a disease, or an individual who has recovered from a disease. In any event, the subject may have been exposed to an antigen characteristic of the disease, such as an antigen capable of producing an antibody immune response associated with the disease. A subject can be a patient. A subject can be a microorganism or microbe (e.g., bacteria, fungi, archaea, viruses).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more nuclei. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitate identification and/or quantification of individual sequencing-reads. In some embodiments, a barcode can be configured for use as a fluorescent barcode. For example, in some embodiments, a barcode can be configured for hybridization to fluorescently labeled oligonucleotide probes. In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotide sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The terms “coupled,” “linked,” “conjugated,” “associated,” “attached,” “connected” or “fused,” as used herein, may be used interchangeably herein and generally refer to one molecule (e.g., polypeptide, receptor, analyte, etc.) being attached or connected (e.g., chemically bound) to another molecule (e.g., polypeptide, receptor, analyte, etc.).

The term “analyte,” in a general sense, refers to a species of interest for detection. An analyte may be biological analyte, such as a nucleic acid molecule or protein, or any appropriate molecule. An analyte may be a secreted analyte, a soluble analyte, and/or an extracellular analyte. It may be intracellular, extracellular, or in association with the cell membrane or nuclear membrane.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Methods of the Disclosure

### Methods for Identifying or Characterizing a Cell as Secreting an Analyte

As described in more detail below, one aspect of the disclosure relates to new approaches and methods for the identification and characterization of a cell or a nucleus as secreting an analyte. The methods for the identification and characterization of the cell as secreting an analyte may detect secretion of the analyte by a cell, characterize the analyte secretion profile of a cell, characterize or identify a cell as secreting the analyte, identify a cell as a cell of interest, or enrich for a cell as a cell of interest. The disclosure also relates to the identification and characterization of a nucleus as secreting an analyte, wherein the nucleus is an isolated nucleus from a cell. Analytes that are transported out of the nucleus via the nuclear membrane are analogous to analytes that are secreted out of a cell via the cell membrane.

In the methods, the secreted analyte may, for example, be a peptide, a nucleic acid, or a lipid molecule analyte. If the secreted analyte is a peptide, it may be a cytokine or a growth factor. For instance, if the secreted analyte is a cytokine, it may be tumor necrosis factor (TNF)α, cluster of differentiation (CD)27, CD30, CD40, interferon (IFN)-α, IFN-β, IFN-γ, IFN-α, IFN-β, IFN-γ, interleukin (IL)-1, IL-2, IL-4, IL-6, IL-10, IL-13, IL-17RA, IL-17RB, IL-17RC, IL17RD, IL-17RE, IL-22, macrophage (M)-colony stimulating factor (CSF), granulocyte (G)M-CSF, or a chemokine. If the secreted analyte is a growth factor, the secreted analyte may be insulin growth factor (IGF), epidermal growth factor (EGF), platelet-derived growth factor (PDGF) or fibroblast growth factor (FGF). If the secreted analyte is a peptide it may also be a hormone or any other analyte secreted in association with a disease, such as inflammatory disease, cancer, or infectious disease. The secreted analyte may also be any analyte that may be expected to be associated with a cell of a particular type that would be useful for its selection and/or enrichment relative to or from other cell types. In some embodiments, if the analyte is a peptide, it is not an antibody, antibody fragment or secreted fragment of a B or T cell receptor. In other embodiments, if the analyte is a peptide, it is an antibody or antigen-binding fragment thereof. Furthermore, if the secreted analyte is a nucleic acid, it may be an RNA, and may be a microRNA. If the secreted analyte is a lipid, it may be a lipid associated with a vesicle, microvesicle or exosome.

In the methods provided herein, a partition may be provided. The partition may also be referred to as a compartment which maintains separation of its own contents from the contents of other partitions. The partition may be a well, a microwell, a flow cell, or a droplet.

The partition may include a cell or a nucleus. The cell included in the partition may be any type of cell, e.g., an immune cell or a tumor. The cell may be one whose secretion profile is to be understood, or it may be a cell suspected of secreting the analyte, or it may be a cell that is to be identified for selection or enrichment if it secretes the analyte. If the cell is an immune cell, it may be a T cell, a B cell, or a dendritic cell. If the cell is an immune cell, it may be suspected of being dysregulated, in the case of a cell of an individual having an inflammatory or autoimmune disease. If the cell is a tumor cell, it may be a lung cancer, pancreatic cancer, liver cancer, breast cancer, prostate cancer or skin cancer cell. The cell may also be an engineered cell or a genetically manipulated cell, e.g., a cell engineered or genetically manipulated to secrete an antibody or antigen-binding fragment thereof. The partition may include a nucleus (instead of the cell) isolated from any of the types of cells described in the preceding disclosure.

Furthermore, the cell may have been obtained from a sample of cells from a subject suspected of having, who has, or may be predisposed to, an infectious, inflammatory, autoimmune or cancerous disease or disorder. The cell from the sample of the subject may have been obtained by biopsy, core biopsy, needle aspirate, or fine needle aspirate. The sample may be a fluid sample, such as a blood sample, urine sample, or saliva sample. The sample may be a plasma or serum sample. Nuclei may be isolated from any of the types of cells described in the preceding disclosure and processed according to the methods described herein.

The sample of the subject may have undergone processing steps so as to arrive at the cell for inclusion in the partition. The processing steps may include steps such as filtration, selective precipitation, purification, centrifugation, agitation, heating, and/or other processes. For example, a sample may be filtered to remove a contaminant or other materials. In some cases, cells and/or cellular constituents of a sample can be processed to separate and/or sort cells of different types. A separation process may be a positive selection process, a negative selection process (e.g., removal of one or more cell types and retention of one or more other cell types of interest), and/or a depletion process (e.g., removal of a single cell type from a sample, such as removal of red blood cells from peripheral blood mononuclear cells).

In the methods provided herein, the provided partition, in addition to the cell, includes a reporter agent. The reporter agent may be for detecting the secreted analyte and may comprise a first aptamer. The first aptamer may include at least two domains. The first aptamer's first domain may bind the analyte. The first aptamer's second domain may comprise: (a) a reporter oligonucleotide or (b) may be responsive to the analyte binding to the first domain. In some embodiments, the first aptamer may include at least three domains: (a) a first domain that binds the analyte, (b) a second domain that comprises a reporter oligonucleotide; and (c) a third domain responsive to analyte binding to the first domain. It will be understood that the cell may have been incubated with the reporter agent prior to being provided in the partition or may first be exposed to the reporter agent once provided in the partition. The partition may include a nucleus (instead of the cell) isolated from the cell described in the preceding disclosure.

If the partition comprises the cell and the reporter agent that: (i) comprises a first aptamer comprising a first domain that binds to the analyte and a second domain comprising a reporter oligonucleotide; or (ii) comprises a first aptamer comprising a first domain that binds to the analyte, a second domain comprising a reporter oligonucleotide and a third domain responsive to the analyte binding to the first domain—then the partition may further comprise a plurality of nucleic acid barcode molecules. The partition may include a nucleus (instead of the cell) isolated from the cell described in the preceding disclosure.

Barcoded nucleic acid molecules may be generated. The barcoded nucleic acid molecules, if generated, may include a sequence of the reporter oligonucleotide or a reverse complement thereof and the partition-specific barcode sequence or reverse complement thereof. The barcoded nucleic acid molecules may be generated in the partition. Alternatively, the barcoded nucleic acid molecules may be generated following (i) coupling of capture sequence(s) of nucleic acid barcode molecule(s) of the plurality nucleic acid barcode molecules to the capture handle sequence(s) of reporter oligonucleotide(s) in their provided partitions and (ii) pooling of the nucleic acid barcode molecule(s) coupled to the capture handle sequences(s) of the reporter oligonucleotide(s) from a plurality of partitions, (e.g., such that the barcoded nucleic acid molecules may be generated in bulk).

If the partition comprises the cell and the reporter agent that: (i) comprises a first aptamer comprising a first domain that binds to the analyte and a second domain responsive to the analyte binding to the first domain or (ii) comprises a first aptamer comprising a first domain that binds to the analyte, a second domain responsive to the analyte binding to the first domain and a third domain comprising a reporter oligonucleotide—then the partition may further include a detectable moiety. Aptamers having domains, or subdomains, responsive to analyte binding can include aptamers that, in response to binding an analyte at the analyte binding domain, undergo a conformational change. The conformational change, in turn, may cause the aptamer to release or take up a dye, e.g. fluorescent dye. The release or uptake of the dye, resulting from the conformational change, e.g., responsive to the analyte binding to the aptamer's analyte binding domain, results in the detectable signal. Examples of aptamers having domains or subdomains responsive to analyte binding, e.g, that undergo a conformational change upon analyte binding that results in detectable uptake or release of a dye, include spinach aptamers, broccoli aptamers, mango aptamers, corn aptamers, and variants thereof, e.g. baby spinach or spinach 2 aptamers. Responsive domains of these example aptamers may be included or engineered, with or without modification, as responsive domains in the aptamers of the reporter agents herein. Responsive domains of these example aptamers may further be used to engineer additional responsive domains that uptake or release new, or further, dyes.

In certain embodiments provided herein, a first aptamer including a domain that is responsive to an analyte binding its first domain may include a subdomain capable of uptake of the detectable moiety upon the analyte binding to the first aptamer's first domain. In other embodiments, a first aptamer including a domain that is responsive to analyte binding its first domain may include a subdomain capable of release of the detectable moiety upon the analyte binding to its first domain. The partition may include a nucleus (instead of the cell) isolated from the cell described in the preceding disclosure.

In methods in which a partition is provided wherein the reporter agent has a domain responsive to the analyte binding to the first, e.g. analyte binding, domain, a detectable signal may be detected from the detectable moiety's uptake or release from the reporter agent. The detectable moiety's detectable signal may be detectable upon release or uptake from the reporter agent. The detectable moiety's detectable signal may be amplified upon uptake or release from the reporter agent. The detectable moiety may be any suitable moiety including a fluorophore, chromophore, heavy metal, radionuclide or any combination thereof. Examples of detectable moieties that can be utilized herein are 3,5-difluoro-4-hydroxybenzylidene imidazolinone (DFHBI), thiazole orange, 3,5-difluoro-4-hydroxynenzylidene imidazolino-2-oxime (DFHO), cyanine (Cy3, Cy5), fluorescein isothiocyanate (FITC), phycoerythrin (PE), allophycocyanin (APC), ALexa Four, and DyLight.

The first aptamer of the reporter agent provided in the partition may additionally be coupled to a lipophilic moiety and/or be capable of binding a cell surface protein, (such as cytoskeleton protein, CD protein or beta actin) or a nuclear membrane protein. If the first aptamer of the reporter agent is coupled to the lipophilic moiety and/or is bound to a cell surface protein or nuclear membrane protein, the first aptamer may be capable of, and may, embed in the cell or nuclear membrane.

The reporter agent, for detecting the analyte in the provided partitions of the methods herein, may also include a second aptamer. In such embodiments, the first aptamer may further include a first linking sequence (e.g., a ligation handle sequence) and the second aptamer may include a second linking sequence (e.g., ligation handle sequence). In such an embodiment, the first linking sequence (e.g., first ligation handle sequence) may be capable of being linked or ligated to the second linking sequence (e.g., second ligation handle sequence), e.g., by complementary base pairing, to thereby link (e.g., ligate) the first aptamer to the second aptamer. The second aptamer may further include a second reporter oligonucleotide and a lipophilic moiety, e.g., to embed the second aptamer in the cell or nuclear membrane. In lieu of, or in addition to, the lipophilic moiety, the second aptamer may be capable of binding a cell surface protein, such as a cytoskeleton protein, a CD protein or beta actin, or a nuclear membrane protein. Linking of the first and second linking sequences may be performed by a number of techniques including, but not limited to, ligation using linking sequences with complementary base pairing or click chemistry using linking sequences having functional groups that can selectively react with each other in an aqueous condition.

Examples of suitable click chemistry reactions can be the Huisgen 1,3-dipolar cycloaddition of an azide and an alkynes, i.e., Copper-catalyzed reaction of an azide with an alkyne to form a 5-membered heteroatom ring called 1,2,3-triazole. The reaction can also be known as a Cu(I)-Catalyzed Azide-Alkyne Cyclo addition (CuAAC), a Cu(I) click chemistry or a Cur click chemistry. Catalyst for the click chemistry can be Cu(I) salts, or Cu(I) salts made in situ by reducing Cu(II) reagent to Cu(I) reagent with a reducing reagent (Pharm Res. 2008, 25(10): 2216-2230). Known Cu(II) reagents for the click chemistry can include, but are not limited to, Cu(II)-(TBTA) complex and Cu(II) (THPTA) complex. TBTA, which is tris-[(1-benzyl-1H-1,2,3-triazol-4-yl)methyl]amine, also known as tris-(benzyltriazolylmethyl)amine, can be a stabilizing ligand for Cu(I) salts. THPTA, which is tris-(hydroxypropyltriazolylmethyl)amine, can be another example of stabilizing agent for Cu(I). Other conditions can also be accomplished to construct the 1,2,3-triazole ring from an azide and an alkyne using copper-free click chemistry, such as by the Strain-promoted Azide-Alkyne Click chemistry reaction (SPAAC, see, e.g., Chem. Commun., 2011, 47:6257-6259 and Nature, 2015, 519(7544):486-90), each of which is entirely incorporated herein by reference for all purposes.

In methods in which the provided partition includes a cell or a nucleus and a reporter agent comprising a first aptamer having a domain including a first reporter oligonucleotide and second aptamer having a domain including a second reporter oligonucleotide, a first and a second barcoded nucleic acid molecule may be generated.

A reporter oligonucleotide, e.g., first or second reporter oligonucleotide, may include: (i) a reporter barcode sequence and (ii) a capture handle sequence. The reporter barcode sequence for each reporter oligonucleotide may be a sequence specific to the aptamer, e.g., the first or second aptamer, in which it is included. For example, if the reporter oligonucleotide is included in a domain of a reporter agent's first aptamer, and the first aptamer is capable of binding to the secreted analyte, then the first reporter oligonucleotide's reporter barcode sequence may specific to the first aptamer, e.g., specific to an aptamer capable of binding to the analyte. In another example, if the reporter oligonucleotide is included in a domain of a reporter agent's second aptamer, and the second aptamer is capable of being linked or ligated to the first aptamer for embedding the reporter agent in the cell or nuclear membrane, then the second reporter oligonucleotide's reporter barcode sequence may specific to the second aptamer, e.g., specific to an aptamer capable of embedding the cell or nuclear membrane. The capture handle sequence, also included in each reporter oligonucleotide with the reporter barcode sequence, may couple to a handle sequence of a first of the plurality of nucleic acid barcode molecules included in the provided partition. The capture handle sequence of the reporter oligonucleotides may couple to the handle sequence of the first of the plurality of nucleic acid barcode molecules, e.g., by complementary base pairing.

In embodiments of the methods in which first and second barcoded nucleic acid molecules are generated, the first barcoded nucleic acid molecule may comprise a sequence of the first reporter oligonucleotide (e.g., reporter barcode sequence specific for the first aptamer, which may be the aptamer capable of binding the secreted analyte or embedding the reporter agent in the cell or nuclear membrane) or a reverse complement thereof and the partition-specific barcode sequence or reverse complement thereof. In embodiments of the methods in which first and second barcoded nucleic acid molecules are generated, the second barcoded nucleic acid molecule may comprise a sequence of the second reporter oligonucleotide (e.g., reporter barcode sequence specific for the second aptamer, which may be capable of the other of binding the secreted analyte or embedding the reporter agent in the cell or nuclear membrane) or a reverse complement thereof and the partition-specific barcode sequence or reverse complement thereof. In such embodiments, if the first and the second barcoded nucleic acid molecules are at approximately a 1:1 ratio, the analyte may be determined to be embedded in the cell or nuclear membrane via the reporter agent. An approximate ratio of 1:1 may be a ratio which the first and the second barcoded nucleic acid molecules are within 80%, 85%, 90%, 95%, 96%, 97%, 98%, or 99% of one another.

It will be understood that in any of the methods in which barcoded nucleic acid molecules are generated, the barcoded nucleic acid molecules may include sequences in addition to sequences of a reporter oligonucleotide or a reverse complement thereof and partition-specific barcode sequence or reverse complement thereof. The barcoded nucleic acid molecules may also include, for example, a unique molecular identifier sequence, or a priming sequence that may originate from the reporter oligonucleotide or one of the plurality of nucleic acid barcode molecules.

In any of the methods in which barcoded nucleic acid molecules are generated, additional operations may be performed using the barcoded nucleic acid molecules. For instance, in one operation, the sequence of the barcoded nucleic acid molecules may be determined. Sequencing may be performed by any of a variety of approaches, systems, or techniques, including next-generation sequencing (NGS) methods. Sequencing may be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, and SMRT® sequencing. Further, sequence analysis of the nucleic acid molecules can be direct or indirect. Thus, the sequence analysis can be performed on a barcoded nucleic acid molecule or it can be a molecule which is derived therefrom (e.g., a complement thereof).

Other examples of methods for sequencing include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing (Oxford Nanopore), Solexa Genome Analyzer sequencing, MS-PET sequencing, whole transcriptome sequencing, and any combinations thereof.

If the determined sequence of the barcoded nucleic acid molecule comprises, e.g., the reporter barcode sequence specific for the aptamer that binds the analyte or a reverse complement thereof, the sequence may identify the cell as secreting the analyte or may characterize the cell as secreting the analyte or may determine secretion level of the analyte by the cell. The cell may instead be a nucleus isolated from the cell described in the preceding disclosure.

In any of the methods in which the reporter agent comprises a first aptamer comprising a domain responsive to the analyte binding to the first aptamer, in which the domain comprises a subdomain that uptakes or releases a detectable moiety upon the first aptamer binding to the analyte, other additional operations may also be performed. The additional operations may include detecting the release or uptake of the detectable moiety, and if the release or uptake of the detectable moiety is detected, identifying the cell as secreting the analyte, characterizing the cell as secreting the analyte, identifying the cell as a cell of interest, or enriching for the cell as a cell of interest. If, as an additional operation, a cell is identified as secreting the analyte, or characterized as secreting the analyte or is identified as a cell of interest, it may be selected for further characterization employing additional reagents as described later herein, using, e.g., transcription analysis, cell surface protein analysis, etc. The cell may instead be a nucleus isolated from the cell described in the preceding disclosure.

By way of example, a cell of interest may be a B regulatory cell and the analyte may be one or more of IL-10, CCL-3 and CCL-4. The cell of interest may be a type of tumor cell and may secrete a selected set of growth factors. Moreover, the cell of interest may be one that responds to a small molecule drug by secreting certain analytes. In such an embodiment, the cell of interest may respond to the small molecule drug in an assay in which the small molecule drug is conjugated to an antigen that mediates transport, e.g., via a flipase mediated pathway or phosphatase, of the small molecule drug into the cell.

In any of the methods provided herein, the cell may be stimulated with one or more agents to stimulate secretion of the analyte. The cell may be stimulated with the one or more agents prior to being provided in the partition. If the cell is stimulated prior to being provided in the partition, it may be stimulated as a cell of a plurality of cells and then partitioned as a single cell into the provided partition. Alternatively, it may be stimulated while isolated as a single cell, then added to a provided partition. If it is stimulated while isolated as a single cell, then added to a provided partition, it may be washed or otherwise treated before being added to the provided partition. The cell may also be stimulated by the addition of the one or more agents following being provided in the partition. The cell may instead be a stimulated nucleus isolated from a cell or a nucleus isolated from a stimulated cell.

The agents mentioned above may include any one or more of feeder cells, a small molecule drug, a cytokine (that is not the secreted analyte), a Pattern recognition receptor (PRR) ligand (e.g., Toll-like receptors (TLR) ligand, NOD-like receptor (NLR) ligand, RIG-I-like receptor (RLR) ligands C-type lectin receptor (CLR) ligand, cytosolic dsDNA sensor (CDS) ligand, etc.), lipopolysaccharide (LPS), double-stranded DNA (dsDNA), double-stranded RNA (dsRNA), a synthetic dsRNA (e.g., polyinosinic-polycytidylic acid (poly I:C) or polyadenylic-polyuridylic acid (poly(A:U)), CpG oligodeoxynucleotides (CpG ODN), or any combination thereof. The agent may be coupled to a major histocompatibility complex (MHC) molecule. In some cases, the MHC molecule may be an MHC multimer (e.g., a monomer, dimer, trimer, tetramer, pentamer, etc.) resulting in a multimeric (e.g., a monomeric, dimeric, trimeric, tetrameric, etc.) WIC-antigen complex (e.g., WIC-peptide complex if the antigen is a peptide). The WIC multimer may be linked to a cell or a polymer. The cell may be an antigen-presenting cell (APC). The polymer may be a dextran polymer (e.g., a dextramer). An MHC multimer (e.g., tetramer or dextramer) or an APC may comprise a plurality of WIC complexes. The MHC molecules/multimers may comprise one or more stimulatory molecules, such as antigenic peptides, thereby forming peptide-MHC complexes. Thus, WIC molecules (e.g., multimers) and/or APCs may be used to present stimulatory and/or co-stimulatory molecules (e.g., stimulatory peptides via MHC-peptide complexes) to the cell (e.g., an immune cell) to induce secretion of the one or more analytes. Moreover, cell or other non-cell constructs may be used to present stimulatory molecules to a cell to induce secretion of one or more analytes. In some cases, an antigen-presenting cell (APC) may comprise a plurality of WIC molecules (e.g., MHC multimers), and thus an APC may be used to induce analyte secretion of a cell (e.g., an immune cell). A co-stimulator molecule as described herein may be an antibody (e.g., an anti-CD3 or an anti-CD28 antibody) or a cytokine (e.g., an interleukin). An APC or an WIC multimer (such as a tetramer or dextramer), for example, may comprise a plurality of co-stimulatory molecules and thus may be used to induce analyte secretion from a cell. In some cases, the stimulator or co-stimulatory molecules, the WIC multimer, the APC, and/or the MHC molecules as described herein may comprise a nucleic acid molecule comprising a barcode sequence. In some cases, methods disclosed herein comprise antigens being part of an antigen-MHC complex (e.g., an antigen-MHC tetramer) comprising the antigen (e.g., a peptide or polypeptide) and an MHC molecule (e.g., an WIC multimer such as a tetramer). The WIC molecule may comprise a nucleic acid molecule comprising a barcode sequence that identifies the peptide(s) present in an WIC molecule or multimer. See, e.g., U.S. Pat. No. 10,011,872.

In particular aspects of any of the embodiments of the methods described herein, the analyte may be an antibody or an antigen-binding fragment thereof. The analyte may an antibody or an antigen-binding fragment thereof and it may be secreted by an antibody-secreting cell. In such methods, the antibody or antigen-binding fragment thereof may further be characterized.

In the methods in which the analyte is an antibody or an antigen-binding fragment thereof, the analyte may be antibody. If the analyte is an antibody, it may be a “full antibody”, as is typically expressed by most mammals including humans, e.g. having four polypeptide chains, two heavy chains (HCs) and two light chains (LCs) inter-connected by disulfide bonds, or a multimer thereof (e.g. IgM). The antibody may be an Immunoglobulin (Ig)A (e.g., IgA1 or IgA2), IgD, IgE, IgG (e.g., IgG1, IgG2, IgG3 and IgG4) or IgM antibody, so long as it can be secreted by a cell.

If the analyte is antigen-binding fragment of an antibody, it may be any naturally occurring, enzymatically obtainable, synthetic, or genetically engineered polypeptide or glycoprotein that specifically binds an antigen to form a complex. Non-limiting examples of an antigen-binding fragment of an antibody include: (i) Fab fragments; (ii) F(ab′)2 fragments; (iii) Fd fragments; (iv) Fv fragments; (v) single-chain Fv (scFv) molecules; (vi) sdAb fragments; and (vii) minimal recognition units consisting of the amino acid residues that mimic the hypervariable region of an antibody (e.g., an isolated complementarity determining region (CDR) such as a CDR3 peptide), or a constrained FWR3-CDR3-FWR4 peptide. Further, an antigen-binding fragment of an antibody may be an engineered molecule, such as a domain-specific antibody, single domain antibody, chimeric antibody, CDR-grafted antibody, diabody, triabody, tetrabody, minibody, nanobody (e.g., monovalent nanobodies, bivalent nanobodies, etc.), a small modular immunopharmaceutical (SMIP), or a shark immunoglobulin new antigen receptor (IgNAR) variable domain.

It is also contemplated that the antigen-binding fragment of the antibody may be a variable domain of an antibody or it may include at least one variable domain of an antibody. The variable domain, of the antigen-binding fragment of the antibody, may be of any size or amino acid composition and may include at least one CDR, which may be adjacent to or in frame with one or more framework sequences. In antigen-binding fragments of antibodies having a VH domain associated with a VL domain, the VH and VL domains may be situated relative to one another in any suitable arrangement. For example, the variable region may be dimeric and may contain VH-VH, VH-VL or VL-VL dimers. Alternatively, the antigen-binding fragment of the antibody may contain a monomeric VH or VL domain. The variable domain may be covalently linked to at least one constant domain. Non-limiting, exemplary configurations of variable and constant domains that can be found within an antigen-binding fragment of an antibody include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) VL-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any of the non-limiting, exemplary configurations of variable and constant domains, the variable and constant domains may be either directly linked to one another or may be linked by a full or partial hinge or linker region. If they are linked by a full or partial hinge region, the hinge region may consist of at least 2 (e.g., 5, 10, 15, 20, 40, 60 or more) amino acids, resulting in a flexible or semi-flexible linkage between adjacent variable and/or constant domains in a single polypeptide molecule. Moreover, an antigen-binding fragment of an antibody may include a homo-dimer or hetero-dimer (or other multimer) of any of the variable and constant domain configurations listed above in non-covalent association with one another and/or with one or more monomeric VH or VL domain (e.g., by disulfide bond(s)).

If the analyte is an antibody, or an antigen-binding fragment thereof, the methods may characterize the antibody or antigen-binding fragment thereof as binding a target antigen. The antibody or antigen-binding fragment thereof may be characterized as binding the target antigen by the inclusion of a second reporter agent in the provided partition; that is, the provided partition including the cell (e.g., antibody-secreting cell) and reporter agent (e.g., including one or more aptamers) may further include the second reporter agent. The second reporter agent may include the target antigen and a reporter oligonucleotide having a sequence specific for the target agent antigen.

The target antigen, that may be included in the second reporter agent, may be an antigen associated with a pathogen or infectious agent, such as a viral, bacterial, parasitic, protozoal or prion agent. If the target antigen is associated with an infectious agent that is a viral agent, the viral agent may be an influenza virus, a coronavirus, a retrovirus, a rhinovirus, or a sarcoma virus. The viral agent may be severe acute respiratory syndrome coronavirus 1 (SARS-CoV-1), a SARS-CoV-2, a Middle East respiratory syndrome coronavirus (MERS-CoV)), or human immunodeficiency virus (HIV), influenza, respiratory syncytial virus, or Ebola virus. If the target antigen is associated with an infectious agent that is a viral agent, the target antigen may be corona virus spike (S) protein, an influenza hemagglutinin protein, an HIV envelope protein or any other a viral glycoprotein. Further, the target antigen may be associated with a tumor or a cancer. If the target agent is associated with a tumor or cancer, it may be, for example, a growth factor or a growth factor receptor. Examples of target antigens that may be associated with tumors or cancers include epidermal growth factor receptor (EGFR), CD38, platelet-derived growth factor receptor (PDGFR) alpha, insulin growth factor receptor (IGFR), CD20, CD19, CD47, or human epidermal growth factor receptor 2 (HER2). Alternatively, the target antigen may be an immune checkpoint molecule that may or may not be associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule or an ion channel. Other examples of a target antigen include autoantigens, virus-like particles and lipoparticles. Further still, the target antigen may be associated with a degenerative condition or disease (e.g., an amyloid protein or a tau protein).

The target antigen that may be included in the second reporter agent may be of a length of at least 20 amino acid residues, at least 40 amino acid residues, at least 60 amino acid residues, at least 80 amino acid residues, at least 100 amino acid residues, at least 200 amino acid residues, at least 300 amino acid residues, at least 400 amino acid residues, at least 500 amino acid residues, at least 600 amino acid residues, at least 700 amino acids, at least 800 amino acid residues, at least 900 amino acid residues, at least 1000 amino acid residues, at least 1100 amino acid residues, at least 1200 amino acid residues, at least 1300 amino acid residues, up to 40 amino acid residues, up to 60 amino acid residues, up to 80 amino acid residues, up to 100 amino acid residues, up to 200 amino acid residues, up to 300 amino acid residues, up to 400 amino acid residues, up to 500 amino acid residues, up to 600 amino acid residues, up to 700 amino acids, up to 800 amino acid residues, up to 900 amino acid residues, up to 1000 amino acid residues, up to 1100 amino acid residues, up to 1200 amino acid residues, or up to 1300 amino acid residues. The target antigen for inclusion in the second reporter agent may be an antigen that includes one domain, at least one domain, two domains, at least two domains, three domains, at least three domains, four domains, at least four domains, five domains, at least five domains, six domains, at least six domains, seven domains, at least seven domains, eight domains, at least eight domains, nine domains, at least nine domains, ten domains, at least ten domains, at least thirty domains, at least forty domains, at least fifty domains, at least sixty domains, at least seventy domains, at least eighty domains, at least ninety domains or at least one hundred domains. The target antigen for inclusion in the second reporter agent may be an antigen that includes at most two hundred domains, at most 175 domains, at most 150 domains, at most 125 domains, at most 100 domains, at most 75 domains, at most 50 domains, at most 25 domains, at most 20 domains, at most 15 domains, at most 10 domains, or at most 5 domains.

The target antigen that may be included in the second reporter agent may further be a protein or peptide as expressed by a cell, e.g., full-length target antigen that may or may not include its leader sequence and may or may not have undergone a similar cell processing step.

In addition to the target antigen, the second reporter agent may include a reporter oligonucleotide. The reporter oligonucleotide may include a reporter barcode sequence, or sequence specific to the target antigen, and a capture handle sequence. The capture handle sequence may couple to a capture sequence of a nucleic acid barcode molecule (of a plurality of nucleic acid barcode molecules) further provided in the partition. Coupling of the capture handle sequence of the reporter oligonucleotide (of the second reporter agent) to the capture sequence of the nucleic acid barcode molecule (of the plurality of nucleic acid barcode molecules) may result in the generation of a barcoded nucleic acid molecule that includes a sequence of the reporter oligonucleotide, or a reverse complement thereof, and the partition-specific barcode sequence or reverse complement thereof, of the nucleic acid barcode molecule.

The generated barcoded nucleic acid molecule including the sequence of the reporter oligonucleotide, or a reverse complement thereof, (specific to the target analyte of the second reporter agent) and the partition-specific barcode sequence or reverse complement thereof (of the nucleic acid barcode molecule) may be sequenced, and the sequence of barcoded nucleic acid molecule may characterize the antibody-secreting cell as binding to, or having affinity for, the target antigen. Sequencing of barcoded nucleic acid molecules has been discussed in this section, e.g., paragraph 85.

Furthermore, the antibody, or an antigen-binding fragment thereof, secreted by an antibody-secreting cell, may be characterized by determining a sequence of, or a sequence encoding, the antibody or antigen-binding fragment thereof. The sequence of, or encoding, the antibody or antigen-binding fragment thereof, may be determined from generation of an additional barcoded nucleic acid molecule. The additional barcoded nucleic acid molecule may be generated using an additional nucleic acid barcode molecule of the plurality of nucleic acid barcode molecules that had been provided in the partition.

The additional nucleic acid barcode molecule, of the plurality of nucleic acid barcode molecules, may include the partition-specific barcode sequence, (identifying the partition in which, for example, the additional nucleic acid barcode molecule had been provided and in which, for other example, the additional barcoded nucleic acid molecule may be generated), and a capture sequence configured to couple to an mRNA or a DNA analyte including sequences encoding the antibody or antigen-binding fragment thereof. If the capture sequence is configured to couple to the mRNA analyte, it may include a polyT sequence. Alternatively, if the capture sequence is configured to couple to the DNA analyte, the DNA analyte may be a cDNA analyte, and the capture sequence may be configured to couple to non-templated nucleotides appended to the cDNA when reverse transcribed from an mRNA analyte. In this capture sequence configuration, the cDNA may be reverse transcribed from the mRNA using a primer having a polydT sequence, and non-templated nucleotides (e.g., one, or at least one, or two, or at least two, or three, or at least three cytosines) may be appended to the cDNA during reverse transcription. The non-templated nucleotides appended to the cDNA during reverse transcription may couple the capture sequence of the nucleic acid barcode molecules (e.g., one, or at least one, or two, or at least two, or three, or at least three guanines) via complementary base pairing. Coupling of the non-templated nucleotide(s) appended to the cDNA to the capture sequence of the nucleic acid barcode molecules extends reverse transcription of the cDNA into the nucleic acid barcode molecules of the plurality of nucleic acid barcode molecules.

The coupling of the capture sequence of the additional nucleic acid barcode molecule to the mRNA or DNA analyte encoding sequences of the antibody or antigen-binding fragment thereof may generate the additional barcoded nucleic acid molecule, e.g, additional barcoded nucleic acid molecule including the partition-specific barcode sequence, or reverse complement thereof, and nucleic acid sequence encoding (or of) the antibody, or antigen-binding fragment thereof. The additional barcoded nucleic acid molecule may be generated in the partition. Alternatively, the additional generated barcoded nucleic acid molecule may be generated in a bulk reaction, following: (a) coupling of the capture sequence of the additional nucleic acid barcode molecule to the mRNA or DNA analyte encoding sequences of the antibody, or antigen-binding fragment thereof in the partition; and (b) pooling of the additional nucleic acid barcode molecules coupled, by their capture sequences, to the to mRNA or cDNA analytes encoding sequences of the antibodies or antigen-binding fragments thereof of multiple partitions. From the generated additional barcoded nucleic acid molecule, sequences of or encoding the antibody, or antigen-binding fragment thereof, may be determined.

The determined sequence of the antibody or antigen-binding fragment thereof, from the additional barcoded nucleic acid molecule, may identify the antibody or antigen-binding fragment thereof. The determined sequences of the antibody or antigen-binding fragment thereof may be nucleic acid sequences encoding the antibody or the antigen-binding fragment thereof. The nucleic acid sequences may encode one or more of a complementarity determining region (CDR), a framework (FWR), a variable heavy chain domain (VH), or a variable light chain domain (VL) of the antibody or antigen-binding fragment thereof. Alternatively, the determined sequences may be amino acid sequences of the antibody or antigen-binding fragment thereof. The amino acid sequences may include a sequence of one or more of a CDR, FWR, VH or VL of the antibody or antigen binding fragment thereof. Sequencing of barcoded nucleic acid molecules was discussed within this section earlier herein, e.g., paragraph 85.

It will also be understood that more than one secreted analyte may be detected and/or analyzed in any of the methods provided herein. For example, in the methods, a first reporter agent may be used to detect a first secreted analyte and a second reporter agent may be used to detect a second secreted analyte. Optionally, at least a second, at least a third, at least a fourth, at least a fifth, at least a sixth, at least a seventh, at least an eighth, at least a ninth, at least a tenth, at least a fifteenth, at least a twentieth, at least a twenty fifth, at least a thirtieth, at least a fiftieth, at least a one hundredth additional reporter agent may be used to detect at least a second, at least a third, at least a fourth, at least a fifth, at least a sixth, at least a seventh, at least an eighth, at least a ninth, at least a tenth, at least a fifteenth, at least a twentieth, at least a twenty fifth, at least a thirtieth, at least a fiftieth, at least a one hundredth additional secreted analyte, respectively. Furthermore, additional reporter agents not including one or more aptamers may be used to detect cell analytes that may, but are not necessarily, secreted by the cell. Such analytes may be cell surface associated, may be nucleic acids comprised in the cell, e.g., mRNA, or lipids and may be detected by reagents described in the “Further Disclosure—Partitions, Partitioning, Reagents and Processing” section herein. The analytes from the cell may instead be analytes from a nucleus isolated from a cell.

### Partitions

In an aspect, the disclosure provides for a partition. It also, in the methods described herein, provides a partition. In a general sense, a “partition,” may be understood to, and may in embodiments disclosed herein, refer to a space or volume that can be suitable to contain one or more cells or nuclei, one or more species of features or compounds, or conduct one or more reactions. Examples of partitions include droplets or microwells.

A partition may include a cell secreting an analyte, suspected of secreting an analyte, or stimulated to secrete an analyte. The cell may be any type of cell, e.g., an immune cell or a tumor. If the cell is an immune cell, it may be a T cell, a B cell, or a dendritic cell. If the cell is a T cell or a B cell, in some embodiments, the analyte is not an antibody, a fragment of an antibody or any portion or whole of a B or T cell receptor. In other embodiments, if the cell is a B cell, the analyte may be an antibody or an antigen-binding fragment thereof. The cell may be a tumor cell such as a lung cancer, pancreatic cancer, liver cancer, breast cancer, prostate cancer or skin cancer cell. The cell may be a cell suspected of being a dysregulated immune cell, in the case of an inflammatory or autoimmune disease. The cell may also be an engineered cell or a genetically manipulated cell, e.g., a cell engineered or genetically manipulated to secrete an antibody or antigen-binding fragment thereof. The partition may instead include a nucleus isolated from a cell.

The cell may be from a sample of cells from a subject. The subject may have or may be suspected of having an infectious, inflammatory, autoimmune or cancer disease or disorder. The cell from the sample of the subject may have been obtained by biopsy, core biopsy, needle aspirate, or fine needle aspirate. The sample may be a fluid sample, such as a blood sample, urine sample, or saliva sample. The sample may be a plasma or serum sample. Nuclei may be isolated from any of these types of cells and may be processed according to the methods described herein.

The sample of the subject, from which the cell may have obtained, may have been subject to processing steps so as to arrive at the cell for inclusion in the partition. The processing steps may include steps such as filtration, selective precipitation, purification, centrifugation, agitation, heating, and/or other processes. For example, a sample may be filtered to remove a contaminant or other materials. In some cases, cells and/or cellular constituents of a sample can be processed to separate and/or sort cells of different types. A separation process may be a positive selection process, a negative selection process (e.g., removal of one or more cell types and retention of one or more other cell types of interest), and/or a depletion process (e.g., removal of a single cell type from a sample, such as removal of red blood cells from peripheral blood mononuclear cells).

The analyte that the cell may be secreting, suspected of secreting or stimulated to secrete may be a peptide, a nucleic acid, or a lipid molecule analyte. If the secreted analyte is a peptide, it may be a cytokine or a growth factor. For instance, if the secreted analyte is a cytokine, it may be tumor necrosis factor (TNF)α, cluster of differentiation (CD)27, CD30, CD40, interferon (IFN)-α, IFN-γ, IFN-α, IFN-γ, interleukin (IL)-1, IL-2, IL-4, IL-6, IL-10, IL-13, IL-17RA, IL-17RB, IL-17RC, IL17RD, IL-17RE, IL-22, macrophage (M)-colony stimulating factor (CSF), granulocyte (G)M-CSF, or a chemokine. If the secreted analyte is a growth factor, the secreted analyte may be insulin growth factor (IGF), epidermal growth factor (EGF), platelet-derived growth factor (PDGF) or fibroblast growth factor (FGF). If the secreted analyte is a peptide it may also be a hormone or any other analyte secreted by a cell associated with a disease, such as inflammatory disease, cancer, or infectious disease. The secreted analyte may also be any analyte that may expected to be associated with a cell of a particular type that would be useful for its selection and/or enrichment relative to or from other cell types. In some embodiments, if the analyte is a peptide, it is not an antibody, antibody fragment or secreted fragment of a B or T cell receptor. In other embodiments, if the analyte is a peptide, it is an antibody or an antigen-binding fragment thereof. Furthermore, if the secreted analyte is a nucleic acid, it may be an RNA, and may be a microRNA. If the secreted analyte is a lipid, it may be a lipid associated with a vesicle, microvesicle or exosome. The cell may instead be a nucleus isolated from a cell.

The partition, in addition to the cell or nucleus, may include a reporter agent that comprises a first aptamer. The first aptamer may include a first domain that binds an analyte and a second domain that is responsive to the analyte binding to the first domain. In some embodiments, the second domain includes a subdomain capable of mediating uptake of a detectable moiety upon binding of the analyte to the first domain. In other embodiments, the second domain includes a subdomain capable of mediating release of the detectable moiety upon binding of the analyte to the first domain.

The first aptamer may further include a third domain that includes a first reporter oligonucleotide. If the first aptamer further includes the third domain, the first reporter oligonucleotide may further include a first reporter barcode sequence specific for the first aptamer (e.g., specific for the binding of the first aptamer to the analyte) and a capture handle sequence.

In some embodiments, the first aptamer of the reporter agent may further be coupled to a lipophilic moiety. In other embodiments, the first aptamer of the reporter agent may include a cell or nucleus binding domain. In yet further embodiments, the first aptamer is both coupled to a lipophilic moiety and includes a cell or nucleus binding domain. If the first aptamer further includes the binding domain, the binding domain may be capable of binding to a cell or nuclear membrane protein, such as a cytoskeleton protein or a cluster of differentiation protein, e.g., CD45, or beta actin.

The reporter agent in the partition with the cell or nucleus may further include a second aptamer. If the reporter agent includes the second aptamer, in addition to the first aptamer, the first aptamer may further include a first linking sequence (e.g., first ligation handle sequence) and the second aptamer may include a second linking sequence (e.g., second ligation handle sequence), wherein the first linking sequence may be capable of linking (e.g., ligating) to the second linking sequence, e.g., by complementary base pairing, to thereby link (e.g., ligate) the first aptamer to the second aptamer. The second aptamer may further include a second reporter oligonucleotide and a lipophilic moiety that embeds the second aptamer in the cell or nuclear membrane. If the second aptamer includes the second reporter oligonucleotide, the second reporter oligonucleotide may include a second reporter barcode specific for the second aptamer (e.g., specific for the second aptamer embedding in the cell or nuclear membrane) sequence and a capture handle sequence. The second aptamer may also be capable of binding a cell surface protein, such as a cytoskeleton protein, a CD protein or beta actin, or a nuclear membrane protein.

In the partition, the first aptamer of the reporter agent may or may not be bound to the analyte. If the reporter agent is bound to the analyte, it may be bound to the analyte external to the cell or nucleus and it may not be coupled to the cell or nucleus. If the reporter agent is bound to the analyte, it may be bound to the analyte external to the cell or nucleus and be coupled to the cell or nucleus, e.g., via the first aptamer being embedded in the cell or nuclear membrane and bound to the analyte at its first domain or via the first aptamer being bound to the analyte at its first domain and linked, e.g., ligated, to the second aptamer while the second aptamer is embedded in the cell or nuclear membrane.

The partition may additionally include a plurality of nucleic acid barcode molecules. A first of the plurality may include a partition-specific barcode sequence and a capture sequence configured to couple to the capture handle sequence of the first and the second reporter oligonucleotide. The capture sequence may be configured to couple to the capture handle sequence of the first and the second reporter oligonucleotide by complementary base pairing.

The partition may additionally include a detectable moiety that the first aptamer may be capable of mediating uptake or release of upon binding of the analyte to the first aptamer's first domain. The detectable moiety may have a detectable signal upon uptake or release from the first aptamer of the reporter agent. The detectable moiety's detectable signal may be amplified upon uptake or release from the first aptamer of the reporter agent. The detectable moiety may be a fluorophore, chromophore, heavy metal, radionuclide or any combinations thereof. Examples of fluorophore that can be utilized here are 3,5-difluoro-4-hydroxybenzylidene imidazolinone (DFHBI), thiazole orange, 3,5-difluoro-4-hydroxynenzylidene imidazolino-2-oxime (DFHO), cyanine (Cy3, Cy5), fluorescein isothiocyanate (FITC), phycoerythrin (PE), allophycocyanin (APC), ALexa Four, DyLight, and etc.

The partition may additionally include one or more agents to stimulate or inhibit secretion of the analyte. The agents include any one or more of feeder cells, a small molecule drug, a cytokine (that is not the secreted analyte), a Pattern recognition receptor (PRR) ligand (e.g., Toll-like receptor (TLR) ligand, NOD-like receptor (NLR) ligand, RIG-I-like receptor (RLR) ligands C-type lectin receptor (CLR) ligand, cytosolic dsDNA sensor (CDS) ligand, etc.), lipopolysaccharide (LPS), double-stranded DNA (dsDNA), double-stranded RNA (dsRNA), a synthetic dsRNA (e.g., polyinosinic-polycytidylic acid (poly I:C) or polyadenylic-polyuridylic acid (poly(A:U)), CpG oligodeoxynucleotides (CpG ODN), or any combination thereof. The agent may be coupled to a major histocompatibility complex (MHC) molecule. In some cases, the MHC molecule may be an MHC multimer (e.g., a monomer, dimer, trimer, tetramer, pentamer, etc.) resulting in a multimeric (e.g., a monomeric, dimeric, trimeric, tetrameric, etc.) MHC-antigen complex (e.g., MHC-peptide complex if the antigen is a peptide). The MHC multimer may be linked to a cell or a polymer. The cell may be an antigen-presenting cell (APC). The polymer may be a dextran polymer (e.g., a dextramer). An MHC multimer (e.g., tetramer or dextramer) or an APC may comprise a plurality of MHC complexes. The MHC molecules/multimers may comprise one or more stimulatory molecules, such as antigenic peptides, thereby forming peptide-MHC complexes. Thus, WIC molecules (e.g., multimers) and/or APCs may be used to present stimulatory and/or co-stimulatory molecules (e.g., stimulatory peptides via MHC-peptide complexes) to the cell (e.g., an immune cell) to induce secretion of the one or more analytes. Moreover, cell or other non-cell constructs may be used to present stimulatory molecules to a cell to induce secretion of one or more analytes. In some cases, an antigen-presenting cell (APC) may comprise a plurality of MI-1C molecules (e.g., MHC multimers), and thus an APC may be used to induce analyte secretion of a cell (e.g., an immune cell). A co-stimulator molecule as described herein may be an antibody (e.g., an anti-CD3 or an anti-CD28 antibody) or a cytokine (e.g., an interleukin). An APC or an WIC multimer (such as a tetramer or dextramer), for example, may comprise a plurality of co-stimulatory molecules and thus may be used to induce analyte secretion from a cell. In some cases, the stimulator or co-stimulatory molecules, the WIC multimer, the APC, and/or the MHC molecules as described herein may comprise a nucleic acid molecule comprising a barcode sequence. In some cases, methods disclosed herein comprise antigens being part of an antigen-MHC complex (e.g., an antigen-MHC tetramer) comprising the antigen (e.g., a peptide or polypeptide) and an MHC molecule (e.g., an WIC multimer such as a tetramer). The WIC molecule may comprise a nucleic acid molecule comprising a barcode sequence that identifies the peptide(s) present in an WIC molecule or multimer. See, e.g., U.S. Pat. No. 10,011,872.

In certain particular embodiments of the partitions described herein above, the partition may further include a second reporter agent. If the second reporter agent is included in the partition, the cell in the partition may be an antibody-secreting cell and the analyte may be an antibody or an antigen-binding fragment thereof. The second reporter agent, included in such a partition, may include a target antigen, e.g., target antigen to which the antibody or antigen-binding fragment thereof may bind, and a reporter oligonucleotide. The reporter oligonucleotide may include a reporter barcode sequence that is specific to the target antigen and may include a capture handle sequence. The capture handle sequence may couple to a barcode nucleic acid molecule, e.g., the first barcode nucleic acid molecule of the plurality of barcode nucleic acid molecules in the partition as discussed herein, e.g., paragraph 117. Further disclosure related to the second reporter agent, target antigens, antibodies and antigen-binding fragments thereof can be found in “METHODS OF THE DISCLOSURE” under “Methods for identifying or characterizing a cell as secreting an analyte” earlier herein.

In addition, the partition may include at least a second, at least a third, at least a fourth, at least a fifth, at least a sixth, at least a seventh, at least an eighth, at least a ninth, at least a tenth, at least a fifteenth, at least a twentieth, at least a twenty fifth, at least a thirtieth, at least a fiftieth, at least a one hundredth other or additional reporter agent that may be used to detect at least a second, at least a third, at least a fourth, at least a fifth, at least a sixth, at least a seventh, at least an eighth, at least a ninth, at least a tenth, at least a fifteenth, at least a twentieth, at least a twenty fifth, at least a thirtieth, at least a fiftieth, at least a one hundredth additional secreted analyte, respectively. Furthermore, the partition may include additional reporter agents, not including one or more aptamers, that may be used to detect cell or nuclear analytes that may, but are not necessarily, secreted by the cell or nucleus. Such analytes may be cell surface or nuclear membrane associated, or may be nucleic acids comprised in the cell, e.g., mRNA, or lipids and may be detected by reagents described in the “Further Disclosure—Partitions, Partitioning, Reagents and Processing” section herein.

### Further Disclosure—Partitions, Partitioning, Reagents and Processing

### Systems and Methods for Partitioning

In some aspects, such as those that have been described above, the methods provided herein include a step of partitioning, or include a step of generating barcoded nucleic acid molecules, or may include an additional processing step(s). In some aspects, the methods herein provide for a partition. This description sets forth examples, embodiments and characteristics of steps of the methods, of the partitions, and of reagents useful in the methods or as may be provided in the partitions.

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions.

In some embodiments disclosed herein, the partitioned particle is a labelled cell, e.g., tumor cell or immune cell. The partitioned cell may be have been stimulated prior to having been partitioned. The partitioned cell may be stimulated by addition of reagents following its partitioning. The labelled cell may instead be a labelled nucleus previously isolated from a cell.

The term “partition,” as used herein, generally, refers to a space or volume that can be suitable to contain one or more cells or nuclei, one or more species of features or compounds, or conduct one or more reactions. A partition can be a physical container, compartment, or vessel, such as a droplet, a flow cell, a reaction chamber, a reaction compartment, a tube, a well, or a microwell. In some embodiments, the compartments or partitions include partitions that are flowable within fluid streams. These partitions can include, for example, micro-vesicles that have an outer barrier surrounding an inner fluid center or core, or, in some cases, the partitions can include a porous matrix that is capable of entraining and/or retaining materials within its matrix. In some aspects, partitions comprise droplets of aqueous fluid within a non-aqueous continuous phase (e.g., oil phase). A variety of different vessels are described in, for example, U.S. Patent Application Publication No. 2014/0155295. Emulsion systems for creating stable droplets in non-aqueous or oil continuous phases are described in detail in, e.g., U.S. Patent Application Publication No. 2010/010511.

In some embodiments, a partition herein includes a space or volume that can be suitable to contain one or more species or conduct one or more reactions. A partition can be a physical compartment, such as a droplet or well. The partition can be an isolated space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

In some embodiments, the methods described herein provide for the compartmentalization, depositing or partitioning of individual cells or nuclei from a sample material containing cells or nuclei, into discrete partitions, where each partition maintains separation of its own contents from the contents of other partitions. Identifiers including unique identifiers (e.g., UMI) and common or universal tags, e.g., barcodes, can be previously, subsequently or concurrently delivered to the partitions that hold the compartmentalized or partitioned cells or nuclei, in order to allow for the later attribution of the characteristics of the individual cells or nuclei to one or more particular compartments. Further, identifiers including unique identifiers and common or universal tags, e.g., barcodes, can be coupled to labelling agents and previously, subsequently or concurrently delivered to the partitions that hold the compartmentalized or partitioned cells or nuclei, in order to allow for the later attribution of the characteristics of the individual cells or nuclei to one or more particular compartments. Identifiers including unique identifiers and common or universal tags, e.g., barcodes, can be delivered, for example on an oligonucleotide, to a partition via any suitable mechanism, for example by coupling the barcoded oligonucleotides to a bead. In some embodiments, the barcoded oligonucleotides are reversibly (e.g., releasably) coupled to a bead. The bead suitable for the compositions and methods of the disclosure can have different surface chemistries and/or physical volumes. In some embodiments, the bead includes a polymer gel. In some embodiments, the polymer gel is a polyacrylamide. Additional non-limiting examples of suitable beads include microparticles, nanoparticles, beads, and microbeads. The partition can be a droplet in an emulsion. A partition can include one or more particles. A partition can include one or more types of particles. For example, a partition of the present disclosure can include one or more biological particles, e.g., labelled engineered cells, tumor cells, immune cells, and/or macromolecular constituents thereof such as nuclei. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition cannot comprise a bead. Unique identifiers, such as barcodes, can be injected into the droplets previous to, subsequent to, or concurrently with droplet generation, such as via a bead, as described elsewhere herein. Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials (e.g., expressed analytes) within its matrix (e.g., via a capture agent configured to couple to both the matrix and the expressed analyte). The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295. Emulsion systems for creating stable droplets in non-aqueous or oil continuous phases are described in, for example, U.S. Patent Application Publication No. 2010/0105112.

In the case of droplets in an emulsion, allocating individual particles (e.g., labelled cells or nuclei) to discrete partitions can, in one non-limiting example, be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions can contain less than one biological particle per partition in order to ensure that those partitions that are occupied are primarily singly occupied. In some cases, partitions among a plurality of partitions can contain at most one biological particle (e.g., bead, DNA, nucleus, or cell, such as a labelled immune or tumor cell). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

In some embodiments, the method further includes individually partitioning one or more single cells or nuclei from a plurality of cells or nuclei in a partition of a second plurality of partitions.

In some embodiments, at least one of the first and second plurality of partitions includes a microwell, a flow cell, a reaction chamber, a reaction compartment, or a droplet. In some embodiments, at least one of the first and second plurality of partitions includes individual droplets in emulsion. In some embodiments, the partitions of the first plurality and/or the second plurality of partition have the same reaction volume.

In the case of droplets in emulsion, allocating individual cells to discrete partitions can generally be accomplished by introducing a flowing stream of cells in an aqueous fluid into a flowing stream of a non-aqueous fluid, such that droplets are generated at the junction of the two streams. By providing the aqueous cell-containing stream at a certain concentration of cells, the occupancy of the resulting partitions (e.g., number of cells per partition) can be controlled. For example, where single cell partitions are desired, the relative flow rates of the fluids can be selected such that, on average, the partitions contain less than one cell per partition, in order to ensure that those partitions that are occupied, are primarily singly occupied. In some embodiments, the relative flow rates of the fluids can be selected such that a majority of partitions are occupied, e.g., allowing for only a small percentage of unoccupied partitions. In some embodiments, the flows and channel architectures are controlled as to ensure a desired number of singly occupied partitions, less than a certain level of unoccupied partitions and less than a certain level of multiply occupied partitions. Individual or single cells may instead be individual or single nuclei isolated from cells.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Application No. PCT/US20/17785, which is entirely incorporated herein by reference for all purposes.

In some embodiments, the methods described herein can be performed such that a majority of occupied partitions include no more than one cell per occupied partition. In some embodiments, the partitioning process is performed such that fewer than 25%, fewer than 20%, fewer than 15%, fewer than 10%, fewer than 5%, fewer than 2%, or fewer than 1% the occupied partitions contain more than one cell. In some embodiments, fewer than 20% of the occupied partitions include more than one cell. In some embodiments, fewer than 10% of the occupied partitions include more than one cell per partition. In some embodiments, fewer than 5% of the occupied partitions include more than one cell per partition. In some embodiments, it is desirable to avoid the creation of excessive numbers of empty partitions. For example, from a cost perspective and/or efficiency perspective, it may be desirable to minimize the number of empty partitions. While this can be accomplished by providing sufficient numbers of cells into the partitioning zone, the Poissonian distribution can optionally be used to increase the number of partitions that include multiple cells. As such, in some embodiments described herein, the flow of one or more of the cells, or other fluids directed into the partitioning zone are performed such that no more than 50% of the generated partitions, no more than 25% of the generated partitions, or no more than 10% of the generated partitions are unoccupied. Further, in some aspects, these flows are controlled so as to present non-Poissonian distribution of single occupied partitions while providing lower levels of unoccupied partitions. Restated, in some aspects, the above noted ranges of unoccupied partitions can be achieved while still providing any of the single occupancy rates described above. For example, in some embodiments, the use of the systems and methods described herein creates resulting partitions that have multiple occupancy rates of less than 25%, less than 20%, less than 15%), less than 10%, and in some embodiments, less than 5%, while having unoccupied partitions of less than 50%), less than 40%, less than 30%, less than 20%, less than 10%, and in some embodiments, less than 5%. Partitioned cells may instead be partitioned nuclei previously isolated from cells.

Although described in terms of providing substantially singly occupied partitions, above, in some embodiments, the methods as described herein include providing multiply occupied partitions, e.g., containing two, three, four or more cells or nuclei and/or beads comprising nucleic acid barcode molecules within a single partition. If more than one cell or nucleus is included in a partition, one cell or nucleus may be the cell or nucleus for which analyte expression and secretion is detected/analyzed and additional cell(s) or nuclei may be provided to stimulate expression and secretion of the analyte by the cell or nucleus.

In some embodiments, the reporter oligonucleotides contained within a partition are distinguishable from the reporter oligonucleotides contained within other partitions of the plurality of partitions.

In some embodiments, it may be desirable to incorporate multiple different barcode sequences within a given partition, either attached to a single or multiple beads within the partition. For example, in some cases, a mixed, but known barcode sequences set can provide greater assurance of identification in the subsequent processing, e.g., by providing a stronger address or attribution of the barcodes to a given partition, as a duplicate or independent confirmation of the output from a given partition.

### Microfluidic Channel Structures

Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids. Methods and systems for generating partitions such as droplets, methods of encapsulating biological particles in partitions, methods of increasing the throughput of droplet generation, and various geometries, architectures, and configurations of microfluidic devices and channels are described in U.S. Patent Publication Nos. 2019/0367997 and 2019/0064173, each of which is entirely incorporated herein by reference for all purposes.

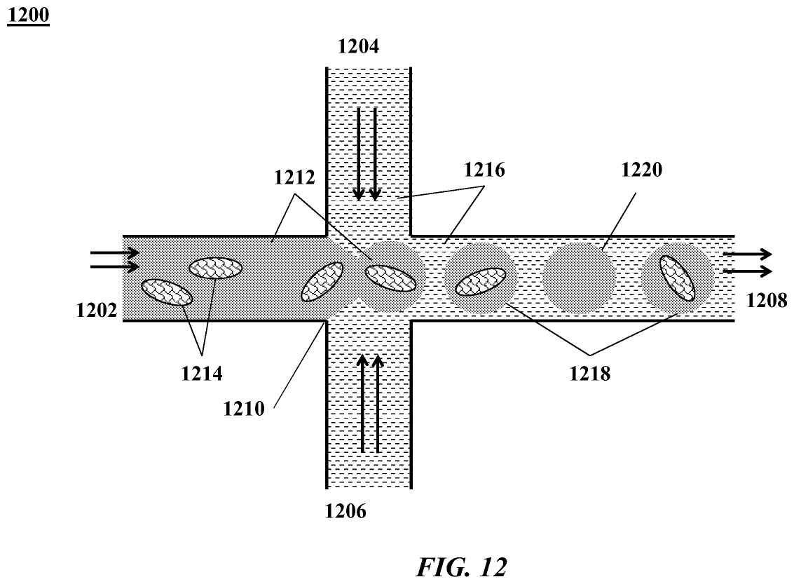

FIG. 12 shows an example of a microfluidic channel structure 1200 for partitioning individual biological particles. The channel structure 1200 can include channel segments 1202, 1204, 1206 and 1208 communicating at a channel junction 1210. In operation, a first aqueous fluid 1212 that includes suspended biological particles (e.g., nuclei or cells, for example, labelled immune or tumor cells) 1214 can be transported along channel segment 1202 into junction 1210, while a second fluid 1216 that is immiscible with the aqueous fluid 1212 is delivered to the junction 1210 from each of channel segments 1204 and 1206 to create discrete droplets 1218, 1220 of the first aqueous fluid 1212 flowing into channel segment 1208, and flowing away from junction 1210. The channel segment 1208 can be fluidically coupled to an outlet reservoir where the discrete droplets can be stored and/or harvested. A discrete droplet generated can include an individual biological particle 1214 (such as droplets 1218). A discrete droplet generated can include more than one individual biological particle (e.g., labelled nuclei or labelled B cells) 1214 (not shown in FIG. 12). A discrete droplet can contain no biological particle 1214 (such as droplet 1220). Each discrete partition can maintain separation of its own contents (e.g., individual biological particle 1214) from the contents of other partitions.

The second fluid 1216 can comprise an oil, such as a fluorinated oil, that includes a fluorosurfactant for stabilizing the resulting droplets, for example, inhibiting subsequent coalescence of the resulting droplets 1218, 1220. Examples of particularly useful partitioning fluids and fluorosurfactants are described, for example, in U.S. Patent Application Publication No. 2010/0105112.

As will be appreciated, the channel segments described herein can be coupled to any of a variety of different fluid sources or receiving components, including reservoirs, tubing, manifolds, or fluidic components of other systems. As will be appreciated, the microfluidic channel structure 1200 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 1218, containing one or more biological particles 1214, e.g., labelled nuclei or cells, e.g., immune or tumor cells, and (2) unoccupied droplets 1220, not containing any biological particles 1214. Occupied droplets 1218 can include singly occupied droplets (having one biological particle, such as one nucleus or cell, e.g., immune or tumor cell) and multiply occupied droplets (having more than one biological particle, such as multiple nuclei or cells, e.g., immune or tumor cells). As described elsewhere herein, in some cases, the majority of occupied partitions can include no more than one biological particle, e.g., labelled nuclei or cells, e.g., immune or tumor cells, per occupied partition and some of the generated partitions can be unoccupied (of any biological particle, or nuclei or labelled immune or tumor cells). In some cases, though, some of the occupied partitions can include more than one biological particle, e.g., labelled nuclei or cells, e.g., labelled immune or tumor cells. In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it can be desirable to minimize the creation of excessive numbers of empty partitions, such as to reduce costs and/or increase efficiency. While this minimization can be achieved by providing a sufficient number of biological particles (e.g., biological particles, such as labelled nuclei or cells, e.g., tumor or immune cells 1214) at the partitioning junction 1210, such as to ensure that at least one biological particle is encapsulated in a partition, the Poissonian distribution can expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles, such as nuclei or cells, e.g., tumor or immune cells, (e.g., in channel segment 1202), or other fluids directed into the partitioning junction (e.g., in channel segments 1204, 1206) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

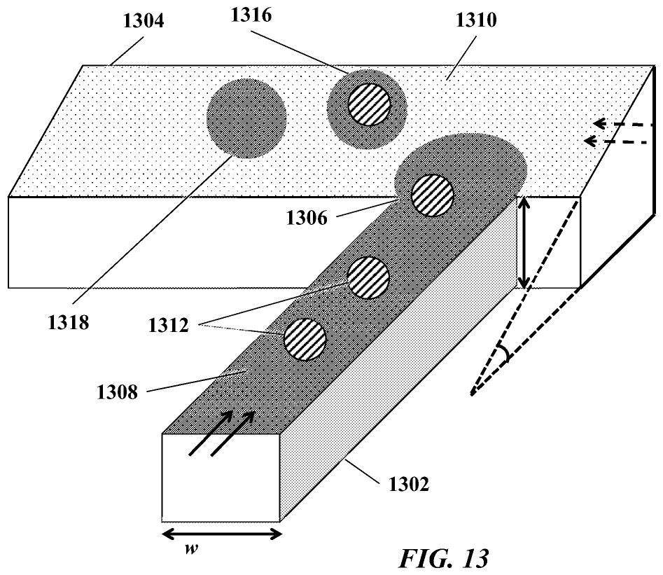

As will be appreciated, the above-described occupancy rates are also applicable to partitions that include both biological particles (e.g., cells or nuclei) and additional reagents, including, but not limited to, beads (e.g., gel beads) carrying nucleic acid barcode molecules (e.g., barcoded oligonucleotides) (described in relation to FIGS. 12 and 13) and/or reporter agents such as reporter agents comprising a first aptamer. The occupied partitions (e.g., at least about 10%, 20%, 30%, 40%, 50%, 60%, 70%, 80%, 90%, 95%, or 99% of the occupied partitions) can include both a bead comprising nucleic acid barcode molecules, a reporter agent and a biological particle.

In another aspect, in addition to or as an alternative to droplet-based partitioning, biological particles (e.g., cells or nuclei) may be encapsulated within a particulate material to form a “cell bead”.

The cell bead can include other reagents. Encapsulation of biological particles, e.g., labelled cells or nuclei, can be performed by a variety of processes. Such processes can combine an aqueous fluid containing the biological particles with a polymeric precursor material that can be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Encapsulation of biological particles, e.g., labelled nuclei or cells, e.g., immune cells, or tumor B cells, can be performed by a variety of methods. For example, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 12, may be readily used in encapsulating biological particles (e.g., cells or nuclei) as described herein. Exemplary methods for encapsulating biological particles (e.g., cells or nuclei) are also further described in U.S. Patent Application Pub. No. US 2015/0376609 and PCT/US2018/016019. In particular, and with reference to FIG. 12, the aqueous fluid 1212 comprising (i) the biological particles 1214 and (ii) the polymer precursor material (not shown) is flowed into channel junction 1210, where it is partitioned into droplets 1218, 1220 through the flow of non-aqueous fluid 1216. In the case of encapsulation methods, non-aqueous fluid 1216 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345.

For example, in the case where the polymer precursor material comprises a linear polymer material, such as a linear polyacrylamide, PEG, or other linear polymeric material, the activation agent can include a cross-linking agent, or a chemical that activates a cross-linking agent within the formed droplets. Likewise, for polymer precursors that comprise polymerizable monomers, the activation agent can include a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) can be provided within the second fluid streams 1216 in channel segments 1204 and 1206, which can initiate the copolymerization of the acrylamide and BAC into a cross-linked polymer network, or hydrogel.

Upon contact of the second fluid stream 1216 with the first fluid stream 1212 at junction 1210, during formation of droplets, the TEMED can diffuse from the second fluid 1216 into the aqueous fluid 1212 comprising the linear polyacrylamide, which will activate the crosslinking of the polyacrylamide within the droplets 1218, 1220, resulting in the formation of gel (e.g., hydrogel) cell beads, as solid or semi-solid beads or particles entraining the nuclei or cells (e.g., immune or tumor cells) 1214. Although described in terms of polyacrylamide encapsulation, other “activatable” encapsulation compositions can also be employed in the context of the methods and compositions described herein. For example, formation of alginate droplets followed by exposure to divalent metal ions (e.g., Ca′ ions), can be used as an encapsulation process using the described processes. Likewise, agarose droplets can also be transformed into capsules through temperature based gelling (e.g., upon cooling, etc.).

In some cases, encapsulated biological particles can be selectively releasable from the cell bead, such as through passage of time or upon application of a particular stimulus, that degrades the encapsulating material sufficiently to allow the biological particles (e.g., labelled nuclei or cells, e.g., immune or tumor cells), or its other contents to be released from the encapsulating material, such as into a partition (e.g., droplet). For example, in the case of the polyacrylamide polymer described above, degradation of the polymer can be accomplished through the introduction of an appropriate reducing agent, such as DTT or the like, to cleave disulfide bonds that cross-link the polymer matrix. See, for example, U.S. Patent Application Publication No. 2014/0378345.

The biological particle (e.g., labelled nuclei or cells, e.g., immune or tumor cells), can be subjected to other conditions sufficient to polymerize or gel the precursors. The conditions sufficient to polymerize or gel the precursors can include exposure to heating, cooling, electromagnetic radiation, and/or light. The conditions sufficient to polymerize or gel the precursors can include any conditions sufficient to polymerize or gel the precursors.

Following polymerization or gelling, a polymer or gel can be formed around the biological particle (e.g., labelled nuclei or cells, e.g., immune or tumor cells). The polymer or gel can be diffusively permeable to chemical or biochemical reagents. The polymer or gel can be diffusively impermeable to macromolecular constituents (e.g., secreted antibodies or antigen-binding fragments thereof) of the biological particle (e.g., labelled nuclei or cells, e.g., immune or tumor cells). In this manner, the polymer or gel can act to allow the biological particle (e.g., labelled nuclei or cells, e.g., immune or tumor cells) to be subjected to chemical or biochemical operations while spatially confining the macromolecular constituents to a region of the droplet defined by the polymer or gel. The polymer or gel can include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel can include any other polymer or gel.

The polymer or gel can be functionalized (e.g., coupled to a capture agent) to bind to targeted analytes (e.g., secreted cytokine or growth factors), such as nucleic acids, proteins, carbohydrates, lipids or other analytes. The polymer or gel can be polymerized or gelled via a passive mechanism. The polymer or gel can be stable in alkaline conditions or at elevated temperature. The polymer or gel can have mechanical properties similar to the mechanical properties of the bead. For instance, the polymer or gel can be of a similar size to the bead. The polymer or gel can have a mechanical strength (e.g., tensile strength) similar to that of the bead. The polymer or gel can be of a lower density than an oil. The polymer or gel can be of a density that is roughly similar to that of a buffer. The polymer or gel can have a tunable pore size. The pore size can be chosen to, for instance, retain denatured nucleic acids. The pore size can be chosen to maintain diffusive permeability to exogenous chemicals such as sodium hydroxide (NaOH) and/or endogenous chemicals such as inhibitors. The polymer or gel can be biocompatible. The polymer or gel can maintain or enhance cell or nucleus viability. The polymer or gel can be biochemically compatible. The polymer or gel can be polymerized and/or depolymerized thermally, chemically, enzymatically, and/or optically.

The polymer can include poly(acrylamide-co-acrylic acid) crosslinked with disulfide linkages. The preparation of the polymer can include a two-step reaction. In the first activation step, poly(acrylamide-co-acrylic acid) can be exposed to an acylating agent to convert carboxylic acids to esters. For instance, the poly(acrylamide-co-acrylic acid) can be exposed to 4-(4,6-dimethoxy-1,3,5-triazin-2-yl)-4-methylmorpholinium chloride (DMTMM). The polyacrylamide-co-acrylic acid can be exposed to other salts of 4-(4,6-dimethoxy-1,3,5-triazin-2-yl)-4-methylmorpholinium. In the second cross-linking step, the ester formed in the first step can be exposed to a disulfide crosslinking agent. For instance, the ester can be exposed to cystamine (2,2′-dithiobis(ethylamine)). Following the two steps, the biological particle can be surrounded by polyacrylamide strands linked together by disulfide bridges. In this manner, the biological particle can be encased inside of or comprise a gel or matrix (e.g., polymer matrix) to form a “cell bead.” A cell bead can contain biological particles (e.g., labelled immune or tumor cells) or macromolecular constituents (e.g., RNA, DNA, proteins, secreted cytokines or growth factors, etc.) of biological particles. A cell bead can include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both (i) cell beads (and/or droplets or other partitions) containing biological particles and (ii) cell beads (and/or droplets or other partitions) containing macromolecular constituents of biological particles.

Encapsulated biological particles (e.g., labelled nuclei or cells, e.g., immune or tumor cells) can provide certain potential advantages of being more storable and more portable than droplet-based partitioned biological particles. Furthermore, in some cases, it can be desirable to allow biological particles (e.g., labelled nuclei or cells, e.g., immune or tumor cells) to incubate for a select period of time before analysis, such as in order to characterize changes in such biological particles over time, either in the presence or absence of different stimuli (e.g., cytokines, antigens, etc.). In such cases, encapsulation can allow for longer incubation than partitioning in emulsion droplets, although in some cases, droplet partitioned biological particles can also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles (e.g., labelled nuclei or cells, e.g., immune or tumor cells) can constitute the partitioning of the biological particles into which other reagents are co-partitioned. Alternatively or in addition, encapsulated biological particles can be readily deposited into other partitions (e.g., droplets) as described above.

### Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well can be configured to hold a volume of about 1000 μL, about 100 μL, about 10 μL, about 1 μL, about 100 nL, about 10 nL, about 1 nL, about 100 pL, about 10 pL, etc. The well can be configured to hold a volume of at least 10 pL, at least 100 pL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 pL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a cell, nucleus, cell bead, or other sample (e.g., cellular or nuclear components, nucleic acid molecules, etc.) while the adjacent microwell can be used to contain a droplet, bead, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, reporter agents, and combinations thereof. The reagents can be physically separated from a sample (for example, a cell, nucleus, cell bead, or cellular components, e.g., proteins, nucleic acid molecules, etc.) that is placed in the well. This physical separation can be accomplished by containing the reagents within, or coupling to, a bead that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

A well can include free reagents and/or reagents encapsulated in, or otherwise coupled to or associated with, beads or droplets. In some embodiments, any of the reagents described in this disclosure can be encapsulated in, or otherwise coupled to, a droplet or bead, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell or nucleus lysis, cell or nucleus fixation, permeabilization, nucleic acid reactions, e.g., nucleic acid extension reactions, amplification, reverse transcription, transposase reactions (e.g., tagmentation), etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell or nucleus lysis, fixation, and/or permeabilization).

In some cases, a well includes a bead or droplet that includes a set of reagents that has a similar attribute, for example, a set of enzymes, a set of minerals, a set of oligonucleotides, a mixture of different barcode molecules, a mixture of identical barcode molecules. In other cases, a bead or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

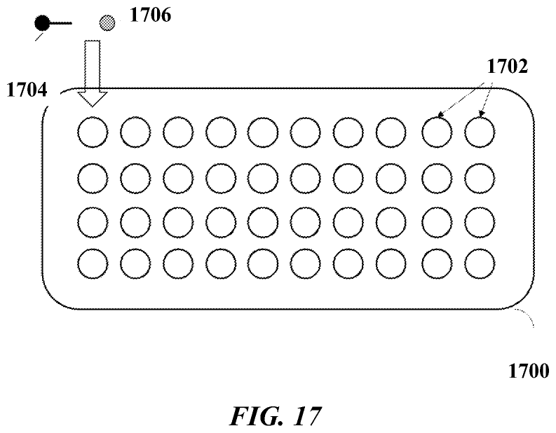

A non-limiting example of a microwell array in accordance with some embodiments of the disclosure is schematically presented in FIG. 17. In this example, the array can be contained within a substrate 1700. The substrate 1700 includes a plurality of wells 1702. The wells 1702 can be of any size or shape, and the spacing between the wells, the number of wells per substrate, as well as the density of the wells on the substrate 1700 can be modified, depending on the particular application. In one such example application, a sample molecule 1706, which can include a cell, nucleus or cellular/nuclear components (e.g., nucleic acid molecules) is co-partitioned with a bead 1704, which can include a nucleic acid barcode molecule coupled thereto. The wells 1702 can be loaded using gravity or other loading technique (e.g., centrifugation, liquid handler, acoustic loading, optoelectronic, etc.). In some instances, at least one of the wells 1702 contains a single sample molecule 1706 (e.g., cell or nucleus) and a single bead 1704.

Reagents can be loaded into a well either sequentially or concurrently. In some cases, reagents are introduced to the device either before or after a particular operation. In some cases, reagents (which can be provided, in certain instances, in droplets or beads) are introduced sequentially such that different reactions or operations occur at different steps. The reagents (or droplets, or beads) can also be loaded at operations interspersed with a reaction or operation step. For example, or droplets or beads including reagents for fragmenting polynucleotides (e.g., restriction enzymes) and/or other enzymes (e.g., transposases, ligases, polymerases, etc.) can be loaded into the well or plurality of wells, followed by loading of droplets or beads including reagents for attaching nucleic acid barcode molecules to a sample nucleic acid molecule. Reagents can be provided concurrently or sequentially with a sample, e.g., a cell, nucleus or cellular/nuclear components (e.g., organelles, proteins, nucleic acid molecules, carbohydrates, lipids, etc.). Accordingly, use of wells can be useful in performing multi-step operations or reactions.

As described elsewhere herein, the nucleic acid barcode molecules and other reagents can be contained within a bead or droplet. These beads or droplets can be loaded into a partition (e.g., a microwell) before, after, or concurrently with the loading of a cell or nucleus, such that each cell or nucleus is contacted with a different bead or droplet. This technique can be used to attach a unique nucleic acid barcode molecule to nucleic acid molecules obtained from each cell or nucleus. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same cell (or nucleus) or partition, while polynucleotides with different barcodes can be determined to originate from different cells (or nuclei) or partitions.

The samples or reagents can be loaded in the wells or microwells using a variety of approaches. For example, the samples (e.g., a cell, nucleus, cell bead, or cellular/nuclear component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single cell or nucleus) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a droplet or bead can be partitioned into a well. The droplets can be selected or subjected to pre-processing prior to loading into a well. For instance, the droplets can include cells or nuclei, and only certain droplets, such as those containing a single cell or nucleus (or at least one cell or nucleus), can be selected for use in loading of the wells. Such a pre-selection process can be useful in efficient loading of single cells or nuclei, such as to obtain a non-Poissonian distribution, or to pre-filter cells or nuclei for a selected characteristic prior to further partitioning in the wells. Additionally, the technique can be useful in obtaining or preventing cell or nucleus doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule within a sample or cell (or nucleus) distributed in the well. For example, the nucleic acid barcode molecules can include a capture sequence that can be used to capture or hybridize to a nucleic acid molecule (e.g., RNA, DNA) within the sample. In some embodiments, the nucleic acid barcode molecules can be releasable from the microwell. For example, the nucleic acid barcode molecules can include a chemical cross-linker which can be cleaved upon application of a stimulus (e.g., photo-, magnetic, chemical, biological, stimulus).

The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In such cases, the unique partition barcode sequences can be used to identify the cell (or nucleus) or partition from which a nucleic acid molecule originated.

Characterization of samples within a well can be performed. Such characterization can include, in non-limiting examples, imaging of the sample (e.g., cell, nucleus, cell bead, or cellular components) or derivatives thereof. Characterization techniques such as microscopy or imaging can be useful in measuring sample profiles in fixed spatial locations. For example, when cells or nuclei are partitioned, optionally with beads, imaging of each microwell and the contents contained therein can provide useful information on cell or nucleus doublet formation (e.g., frequency, spatial locations, etc.), cell-bead pair efficiency, cell (or nucleus) viability, cell (or nucleus) size, cell (or nucleus) morphology, expression level of a biomarker (e.g., a surface marker, a fluorescently labeled molecule therein, etc.), cell, nucleus or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When cells, nuclei or cell beads are loaded, the well can be subjected to washing, e.g., to remove excess cells or nuclei from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells or nuclei can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any suitable or useful step, the well (or microwell array or plate) can be sealed (e.g., using an oil, membrane, wax, etc.), which enables storage of the assay or selective introduction of additional reagents.

Once sealed, the well may be subjected to conditions for further processing of a biological particle (e.g., a cell, a cell bead or a nucleus) in the well. For instance, reagents in the well may allow further processing of the biological particle, e.g., lysis of the cell or nucleus, as further described herein. Alternatively, the well (or wells such as those of a well-based array) comprising the biological particle (e.g., cell, cell bead, or nucleus) may be subjected to freeze-thaw cycling to process the biological particle(s), e.g., lysis of a cell or nucleus. The well containing the biological particle (e.g., cell, cell bead, or nucleus) may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the biological particle(s) (e.g., cell(s), cell bead(s), nucleus or nuclei) may be subjected to freeze thaw cycles to lyse biological particle(s). In one embodiment, the initially frozen well (or wells) are thawed to a temperature above freezing (e.g., room temperature or 25° C.). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, or 4 times, to obtain lysis of the biological particle(s) (e.g., cell(s), cell bead(s), nucleus, or nuclei) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

### Beads

In some embodiments of the disclosure, a partition can include one or more unique identifiers, such as barcodes (e.g., a plurality of nucleic acid barcode molecules which can be, for example, a plurality of partition barcode sequences). Barcodes can be previously, subsequently or concurrently delivered to the partitions that hold the compartmentalized or partitioned biological particle (e.g., labelled nuclei or cells, e.g., immune or tumor cells). For example, barcodes can be injected into droplets previous to, subsequent to, or concurrently with droplet generation. In some embodiments, the delivery of the barcodes to a particular partition allows for the later attribution of the characteristics of the individual biological particle (e.g., labelled nuclei or cells, e.g., immune or tumor cells) to the particular partition. Barcodes can be delivered, for example on a nucleic acid molecule (e.g., a barcoded oligonucleotide), to a partition via any suitable mechanism. In some embodiments, nucleic acid barcode molecules can be delivered to a partition via a bead. Beads are described in further detail below.

In some embodiments, nucleic acid barcode molecules can be initially associated with the bead and then released from the bead. In some embodiments, release of the nucleic acid barcode molecules can be passive (e.g., by diffusion out of the bead). In addition or alternatively, release from the bead can be upon application of a stimulus which allows the nucleic acid barcode molecules to dissociate or to be released from the bead. Such stimulus can disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent), a mechanical stimulus, a radiation stimulus; a biological stimulus (e.g., enzyme), an electrical stimulus, or any combination thereof. Methods and systems for partitioning barcode carrying beads into droplets are provided in US. Patent Publication Nos. 2019/0367997 and 2019/0064173, and International Application Nos. PCT/US20/17785 and PCT/US20/020486.

Beneficially, a discrete droplet partitioning a biological particle and a barcode carrying bead can effectively allow the attribution of the barcode to macromolecular constituents of the biological particle within the partition. The contents of a partition can remain discrete from the contents of other partitions.

In operation, the barcoded oligonucleotides can be released (e.g., in a partition), as described elsewhere herein. Alternatively, the nucleic acid molecules bound to the bead (e.g., gel bead) can be used to hybridize and capture analytes (e.g., one or more types of analytes) on the solid phase of the bead.

In some examples, beads, biological particles (e.g., labelled nuclei or cells, e.g., immune or tumor cells) and droplets can flow along channels (e.g., the channels of a microfluidic device), in some cases at substantially regular flow profiles (e.g., at regular flow rates). Such regular flow profiles can permit a droplet to include a single bead and a single biological particle. Such regular flow profiles can permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that can be used to provide such regular flow profiles are provided in, for example, U.S. Patent Publication No. 2015/0292988.

A bead can be porous, non-porous, solid, semi-solid, semi-fluidic, fluidic, and/or a combination thereof. In some instances, a bead can be dissolvable, disruptable, and/or degradable. In some cases, a bead cannot be degradable. In some cases, the bead can be a gel bead. A gel bead can be a hydrogel bead. A gel bead can be formed from molecular precursors, such as a polymeric or monomeric species. A semi-solid bead can be a liposomal bead. Solid beads can include metals including iron oxide, gold, and silver. In some cases a bead can be a magnetic bead or a paramagnetic bead. In some cases, the bead can be a silica bead. In some cases, the bead can be rigid. In other cases, the bead can be flexible and/or compressible.

A bead can be of any suitable shape. Examples of bead shapes include, but are not limited to, spherical, non-spherical, oval, oblong, amorphous, circular, cylindrical, and variations thereof.

Beads can be of uniform size or heterogeneous size. In some cases, the diameter of a bead can be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead can have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead can have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In some embodiments, the beads described herein can have size distributions that have a coefficient of variation in their cross-sectional dimensions of less than 50%, less than 40%, less than 30%, less than 20%, and in some cases less than 15%, less than 10%, less than 5%, or less.

A bead can include natural and/or synthetic materials. For example, a bead can include a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads can also be formed from materials other than polymers, including lipids, micelles, ceramics, glass-ceramics, material composites, metals, other inorganic materials, and others.

In some embodiments, the bead can contain molecular precursors (e.g., monomers or polymers), which can form a polymer network via polymerization of the molecular precursors. In some cases, a precursor can be an already polymerized species capable of undergoing further polymerization via, for example, a chemical cross-linkage. In some embodiments, a precursor can include one or more of an acrylamide or a methacrylamide monomer, oligomer, or polymer. In some cases, the bead can include prepolymers, which are oligomers capable of further polymerization. For example, polyurethane beads can be prepared using prepolymers. In some embodiments, the bead can contain individual polymers that can be further polymerized together. In some cases, beads can be generated via polymerization of different precursors, such that they include mixed polymers, co-polymers, and/or block co-polymers. In some embodiments, the bead can include covalent or ionic bonds between polymeric precursors (e.g., monomers, oligomers, linear polymers), nucleic acid molecules (e.g., oligonucleotides), primers, and other entities. In some embodiments, the covalent bonds can be carbon-carbon bonds, thioether bonds, or carbon-heteroatom bonds.

Cross-linking can be permanent or reversible, depending upon the particular cross-linker used. Reversible cross-linking can allow for the polymer to linearize or dissociate under appropriate conditions. In some embodiments, reversible cross-linking can also allow for reversible attachment of a material bound to the surface of a bead. In some embodiments, a cross-linker can form disulfide linkages. In some embodiments, the chemical cross-linker forming disulfide linkages can be cystamine or a modified cystamine.

In some embodiments, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent including a disulfide bond that can be used as a crosslinker agent between individual monomeric or polymeric precursors of a bead. Polyacrylamide can be polymerized in the presence of cystamine or a species including cystamine (e.g., a modified cystamine) to generate polyacrylamide gel beads including disulfide linkages (e.g., chemically degradable beads including chemically-reducible cross-linkers). The disulfide linkages can permit the bead to be degraded (or dissolved) upon exposure of the bead to a reducing agent.

In some embodiments, chitosan, a linear polysaccharide polymer, can be crosslinked with glutaraldehyde via hydrophilic chains to form a bead. Crosslinking of chitosan polymers can be achieved by chemical reactions that are initiated by heat, pressure, change in pH, and/or radiation.

In some embodiments, a bead can include an acrydite moiety, which in certain aspects can be used to attach one or more nucleic acid molecules (e.g., barcode sequence, nucleic acid barcode molecule, barcoded oligonucleotide, primer, or other oligonucleotide) to the bead.

In some cases, an acrydite moiety can refer to an acrydite analogue generated from the reaction of acrydite with one or more species, such as, the reaction of acrydite with other monomers and cross-linkers during a polymerization reaction. Acrydite moieties can be modified to form chemical bonds with a species to be attached, such as a nucleic acid molecule (e.g., barcode sequence, nucleic acid barcode molecule, barcoded oligonucleotide, primer, or other oligonucleotide). Acrydite moieties can be modified with thiol groups capable of forming a disulfide bond or can be modified with groups already including a disulfide bond. The thiol or disulfide (via disulfide exchange) can be used as an anchor point for a species to be attached or another part of the acrydite moiety can be used for attachment. In some cases, attachment can be reversible, such that when the disulfide bond is broken (e.g., in the presence of a reducing agent), the attached species is released from the bead. In other cases, an acrydite moiety can include a reactive hydroxyl group that can be used for attachment.

Functionalization of beads for attachment of nucleic acid molecules (e.g., oligonucleotides) can be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead can include acrydite moieties, such that when a bead is generated, the bead also includes acrydite moieties. The acrydite moieties can be attached to a nucleic acid molecule (e.g., oligonucleotide), which can include a priming sequence (e.g., a primer for amplifying target nucleic acids, random primer, primer sequence for messenger RNA) and/or one or more barcode sequences. The one or more barcode sequences can include sequences that are the same for all nucleic acid molecules coupled to a given bead and/or sequences that are different across all nucleic acid molecules coupled to the given bead. The nucleic acid molecule can be incorporated into the bead.

In some embodiments, the nucleic acid molecule, e.g., nucleic acid barcode molecule, can further comprise a unique molecular identifier (UMI). In some embodiments, the nucleic acid molecule can include one or more functional sequences, for example, for attachment to a sequencing flow cell, such as, for example, a P5 sequence (or a portion thereof) for Illumina® sequencing. In some cases, the nucleic acid molecule or derivative thereof (e.g., oligonucleotide or polynucleotide generated from the nucleic acid molecule) can include another functional sequence, such as, for example, a P7 sequence for attachment to a sequencing flow cell for Illumina sequencing. In some cases, the nucleic acid molecule can include a barcode sequence. In some cases, the primer can further include a unique molecular identifier (UMI). In some cases, the primer can include an R1 primer sequence for Illumina sequencing. In some cases, the primer can include an R2 primer sequence for Illumina sequencing. In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Nucleic acid molecules, e.g., nucleic acid barcode molecules, can comprise one or more functional sequences for coupling to an analyte or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

Examples of such nucleic acid molecules (e.g., oligonucleotides, polynucleotides, etc.) and uses thereof, as can be used with compositions, devices, methods and systems of the present disclosure, are provided in U.S. Patent Pub. Nos. 2014/0378345 and 2015/0376609.

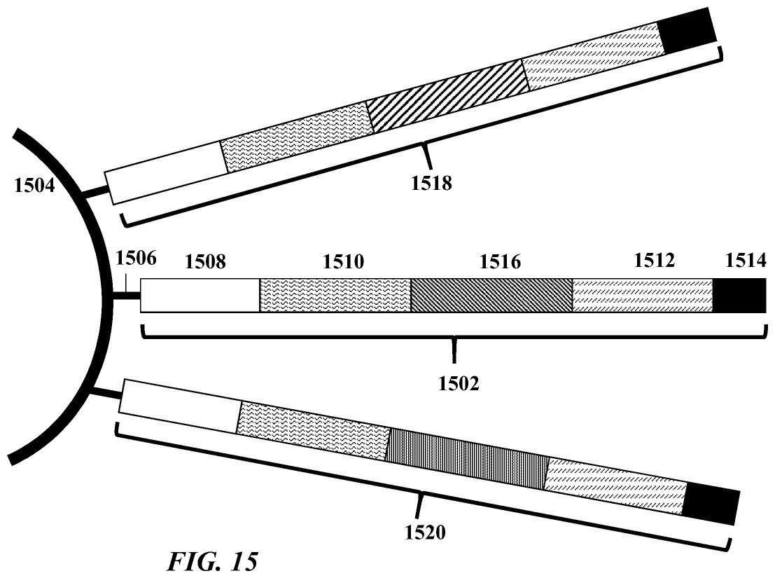

FIG. 15 illustrates an example of a barcode carrying bead. A nucleic acid molecule 1502, such as an oligonucleotide, can be coupled to a bead 1504 by a releasable linkage 1506, such as, for example, a disulfide linker. The same bead 1504 can be coupled (e.g., via releasable linkage) to one or more other nucleic acid molecules 1518, 1520. The nucleic acid molecule 1502 can be or include a barcode. As noted elsewhere herein, the structure of the barcode can include a number of sequence elements. The nucleic acid molecule 1502 can include a functional sequence 1508 that can be used in subsequent processing. For example, the functional sequence 1508 can include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 1502 can include a barcode sequence 1510 for use in barcoding the sample (e.g., DNA, RNA, protein, etc.). In some cases, the barcode sequence 1510 can be bead-specific such that the barcode sequence 1510 identifies nucleic acid molecule 1502 as coupled to the same bead 1504, e.g., is common to all nucleic acid molecules (e.g., including nucleic acid molecule 1502) coupled to the same bead 1504. Alternatively or in addition, the barcode sequence 1510 can be partition-specific such that the barcode sequence 1510 identifies 1502 as having been partitioned into a particular partition, e.g., is common to all nucleic acid molecules coupled to one or more beads that are partitioned into the same partition. The nucleic acid molecule 1502 can include a specific priming sequence 1512, such as an mRNA specific priming sequence (e.g., poly-T sequence), a targeted priming sequence, and/or a random priming sequence. The nucleic acid molecule 1502 can include an anchoring sequence 1514 to ensure that the specific priming sequence 1512 hybridizes at the sequence end (e.g., of the mRNA). For example, the anchoring sequence 1514 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 1502 can include a unique molecular identifying sequence 1516 (e.g., unique molecular identifier (UMI)). In some cases, the unique molecular identifying sequence 1516 can include from about 5 to about 8 nucleotides. Alternatively, the unique molecular identifying sequence 1516 can compress less than about 5 or more than about 8 nucleotides. The unique molecular identifying sequence 1516 can be a unique sequence that varies across individual nucleic acid molecules (e.g., 1502, 1518, 1520, etc.) coupled to a single bead (e.g., bead 1504). In some cases, the unique molecular identifying sequence 1516 can be a random sequence (e.g., such as a random N-mer sequence). For example, the UMI can provide a unique identifier of the starting mRNA molecule that was captured, in order to allow quantitation of the number of original expressed RNA. As will be appreciated, although FIG. 15 shows three nucleic acid molecules 1502, 1518, 1520 coupled to the surface of the bead 1504, an individual bead can be coupled to any number of individual nucleic acid molecules, for example, from one to tens to hundreds of thousands or even millions of individual nucleic acid molecules. The respective barcodes for the individual nucleic acid molecules can include both common sequence segments or relatively common sequence segments (e.g., 1508, 1510, 1512, etc.) and variable or unique sequence segments (e.g., 1516) between different individual nucleic acid molecules coupled to the same bead.

In operation, a biological particle (e.g., cell, nucleus, DNA, RNA, etc.) can be co-partitioned along with a barcode bearing bead 1504. The nucleic acid barcode molecules 1502, 1518, 1520 can be released from the bead 1504 in the partition. By way of example, in the context of analyzing sample RNA, the poly-T segment (e.g., 1512) of one of the released nucleic acid molecules (e.g., 1502) can hybridize to the poly-A tail of a mRNA molecule. Reverse transcription can result in a cDNA transcript of the mRNA, but which transcript includes each of the sequence segments 1508, 1510, 1516 of the nucleic acid molecule 1502. Because the nucleic acid molecule 1502 includes an anchoring sequence 1514, it will more likely hybridize to and prime reverse transcription at the sequence end of the poly-A tail of the mRNA. Within any given partition, all of the cDNA transcripts of the individual mRNA molecules can include a common barcode sequence segment 1510. However, the transcripts made from the different mRNA molecules within a given partition can vary at the unique molecular identifying sequence 1512 segment (e.g., UMI segment). Beneficially, even following any subsequent amplification of the contents of a given partition, the number of different UMIs can be indicative of the quantity of mRNA originating from a given partition, and thus from the biological particle (e.g., cell or nucleus). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences can also be used in priming the reverse transcription reaction. Likewise, although described as releasing the barcoded oligonucleotides into the partition, in some cases, the nucleic acid molecules bound to the bead (e.g., gel bead) can be used to hybridize and capture the mRNA on the solid phase of the bead, for example, in order to facilitate the separation of the RNA from other cell or nucleus contents. In such cases, further processing can be performed, in the partitions or outside the partitions (e.g., in bulk). For instance, the RNA molecules on the beads can be subjected to reverse transcription or other nucleic acid processing, additional adapter sequences can be added to the barcoded nucleic acid molecules, or other nucleic acid reactions (e.g., amplification, nucleic acid extension) can be performed. The beads or products thereof (e.g., barcoded nucleic acid molecules) can be collected from the partitions, and/or pooled together and subsequently subjected to clean up and further characterization (e.g., sequencing).

The operations described herein can be performed at any useful or suitable step. For instance, the beads including nucleic acid barcode molecules can be introduced into a partition (e.g., well or droplet) prior to, during, or following introduction of a sample into the partition. The nucleic acid molecules of a sample can be subjected to barcoding, which can occur on the bead (in cases where the nucleic acid molecules remain coupled to the bead) or following release of the nucleic acid barcode molecules into the partition. In some cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases where the nucleic acid molecules from the sample remain attached to the bead, the beads from various partitions can be collected, pooled, and subjected to further processing (e.g., reverse transcription, adapter attachment, amplification, clean up, and/or sequencing). In other instances, one or more of the processing methods, e.g., reverse transcription, can occur in the partition. For example, conditions sufficient for barcoding, adapter attachment, reverse transcription, or other nucleic acid processing operations can be provided in the partition and performed prior to clean up and sequencing.

In some instances, a bead can include a capture sequence or binding sequence configured to bind to a corresponding capture sequence or binding sequence. In some instances, a bead can include a plurality of different capture sequences or binding sequences configured to bind to different respective corresponding capture sequences or binding sequences. For example, a bead can include a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead can include any number of different capture sequences. In some instances, a bead can include at least 2, 3, 4, 5, 6, 7, 8, 9, 10 or more different capture sequences or binding sequences configured to bind to different respective capture sequences or binding sequences, respectively. Alternatively or in addition, a bead can include at most about 10, 9, 8, 7, 6, 5, 4, 3, or 2 different capture sequences or binding sequences configured to bind to different respective capture sequences or binding sequences. In some instances, the different capture sequences or binding sequences can be configured to facilitate analysis of a same type of analyte. In some instances, the different capture sequences or binding sequences can be configured to facilitate analysis of different types of analytes (with the same bead). The capture sequence can be designed to attach to a corresponding capture sequence. Beneficially, such corresponding capture sequence can be introduced to, or otherwise induced in, a biological particle (e.g., cell, nucleus, cell bead, etc.) for performing different assays in various formats (e.g., barcoded antibodies including the corresponding capture sequence, barcoded MHC dextramers including the corresponding capture sequence, barcoded guide RNA molecules including the corresponding capture sequence, etc.), such that the corresponding capture sequence can later interact with the capture sequence associated with the bead. In some instances, a capture sequence coupled to a bead (or other support) can be configured to attach to a linker molecule, such as a splint molecule, wherein the linker molecule is configured to couple the bead (or other support) to other molecules through the linker molecule, such as to one or more analytes or one or more other linker molecules.

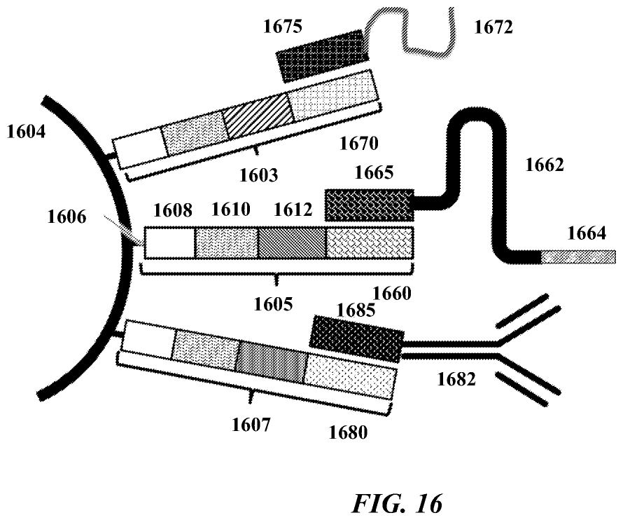

FIG. 16 illustrates a non-limiting example of a barcode carrying bead in accordance with some embodiments of the disclosure. A nucleic acid molecule, e.g., nucleic acid barcode molecule, 1605, such as an oligonucleotide, can be coupled to a bead 1604 by a releasable linkage 1606, such as, for example, a disulfide linker. The nucleic acid molecule 1605 can include a first capture sequence 1660. The same bead 1604 can be coupled, e.g., via releasable linkage, to one or more other nucleic acid molecules 1603, 1607 including other capture sequences. The nucleic acid molecule 1605 can be or include a barcode. As described elsewhere herein, the structure of the barcode can include a number of sequence elements, such as a functional sequence 1608 (e.g., flow cell attachment sequence, sequencing primer sequence, etc.), a barcode sequence 1610 (e.g., bead-specific sequence common to bead, partition-specific sequence common to partition, etc.), and a unique molecular identifier 1612 (e.g., unique sequence within different molecules attached to the bead), or partial sequences thereof. The capture sequence 1660 can be configured to attach to a corresponding capture sequence 1665 (e.g., capture handle). In some instances, the corresponding capture sequence 1665 can be coupled to another molecule that can be an analyte or an intermediary carrier. For example, as illustrated in FIG. 16, the corresponding capture sequence 1665 is coupled to a guide RNA molecule 1662 including a target sequence 1664, wherein the target sequence 1664 is configured to attach to the analyte. Another oligonucleotide molecule 1607 attached to the bead 1604 includes a second capture sequence 1680 which is configured to attach to a second corresponding capture sequence (e.g., capture handle) 1685. As illustrated in FIG. 16, the second corresponding capture sequence 1685 is coupled to an antibody 1682. In some cases, the antibody 1682 can have binding specificity to an analyte (e.g., surface protein). Alternatively, the antibody 1682 cannot have binding specificity. Another oligonucleotide molecule 1603 attached to the bead 1604 includes a third capture sequence 470 which is configured to attach to a second corresponding capture sequence 1675. As illustrated in FIG. 16, the third corresponding capture sequence (e.g., capture handle) 1675 is coupled to a molecule 1672. The molecule 1672 may or may not be configured to target an analyte. The other oligonucleotide molecules 1603, 1607 can include the other sequences (e.g., functional sequence, barcode sequence, UMI, etc.) described with respect to oligonucleotide molecule 1605. While a single oligonucleotide molecule including each capture sequence is illustrated in FIG. 16, it will be appreciated that, for each capture sequence, the bead can include a set of one or more oligonucleotide molecules each including the capture sequence. For example, the bead can include any number of sets of one or more different capture sequences. Alternatively or in addition, the bead 1604 can include other capture sequences. Alternatively or in addition, the bead 1604 can include fewer types of capture sequences (e.g., two capture sequences). Alternatively or in addition, the bead 1604 can include oligonucleotide molecule(s) including a priming sequence, such as a specific priming sequence such as an mRNA specific priming sequence (e.g., poly-T sequence), a targeted priming sequence, and/or a random priming sequence, for example, to facilitate an assay for gene expression.

The generation of a barcoded sequence, see, e.g., FIG. 15, is described herein. In some embodiments, precursors including a functional group that is reactive or capable of being activated such that it becomes reactive can be polymerized with other precursors to generate gel beads including the activated or activatable functional group. The functional group can then be used to attach additional species (e.g., disulfide linkers, primers, other oligonucleotides, etc.) to the gel beads. For example, some precursors including a carboxylic acid (COOH) group can co-polymerize with other precursors to form a gel bead that also includes a COOH functional group. In some cases, acrylic acid (a species including free COOH groups), acrylamide, and bis(acryloyl)cystamine can be co-polymerized together to generate a gel bead including free COOH groups. The COOH groups of the gel bead can be activated (e.g., via 1-Ethyl-3-(3-dimethylaminopropyl)carbodiimide (EDC) and N-Hydroxysuccinimide (NETS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species including an amine functional group where the carboxylic acid groups are activated to be reactive with an amine functional group) including a moiety to be linked to the bead.

Beads including disulfide linkages in their polymeric network can be functionalized with additional species via reduction of some of the disulfide linkages to free thiols. The disulfide linkages can be reduced via, for example, the action of a reducing agent (e.g., DTT, TCEP, etc.) to generate free thiol groups, without dissolution of the bead. Free thiols of the beads can then react with free thiols of a species or a species including another disulfide bond (e.g., via thiol-disulfide exchange) such that the species can be linked to the beads (e.g., via a generated disulfide bond). In some cases, free thiols of the beads can react with any other suitable group. For example, free thiols of the beads can react with species including an acrydite moiety. The free thiol groups of the beads can react with the acrydite via Michael addition chemistry, such that the species including the acrydite is linked to the bead. In some cases, uncontrolled reactions can be prevented by inclusion of a thiol capping agent such as N-ethylmaleimide or iodoacetate.

Activation of disulfide linkages within a bead can be controlled such that only a small number of disulfide linkages are activated. Control can be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent can be used for reduction. Controlling the number of disulfide linkages that are reduced to free thiols can be useful in ensuring bead structural integrity during functionalization. In some cases, optically-active agents, such as fluorescent dyes can be coupled to beads via free thiol groups of the beads and used to quantify the number of free thiols present in a bead and/or track a bead.

In some embodiments, addition of moieties to a gel bead after gel bead formation can be advantageous. For example, addition of an oligonucleotide (e.g., barcoded oligonucleotide, such as a nucleic acid barcode molecule) after gel bead formation can avoid loss of the species during chain transfer termination that can occur during polymerization. Moreover, smaller precursors (e.g., monomers or cross linkers that do not include side chain groups and linked moieties) can be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel can possess an upper critical solution temperature (UCST) that can permit temperature driven swelling and collapse of a bead. Such functionality can aid in oligonucleotide (e.g., a primer) infiltration into the bead during subsequent functionalization of the bead with the oligonucleotide. Post-production functionalization can also be useful in controlling loading ratios of species in beads, such that, for example, the variability in loading ratio is minimized. Species loading can also be performed in a batch process such that a plurality of beads can be functionalized with the species in a single batch.

A bead injected or otherwise introduced into a partition can include releasably, cleavably, or reversibly attached barcodes (e.g., partition barcode sequences). A bead injected or otherwise introduced into a partition can include activatable barcodes. A bead injected or otherwise introduced into a partition can be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage can be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads can be degradable, disruptable, or dissolvable spontaneously or upon exposure to one or more stimuli (e.g., temperature changes, pH changes, exposure to particular chemical species or phase, exposure to light, reducing agent, etc.). In some cases, a bead can be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead can be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) can result in release of the species from the bead.

As will be appreciated from the above disclosure, the degradation of a bead can refer to the disassociation of a bound (e.g., capture agent configured to couple to a secreted antibody or antigen-binding fragment thereof) or entrained species (e.g., labelled nuclei or cells, e.g., immune cells or tumor cells, or a secreted analyte) from a bead, both with and without structurally degrading the physical bead itself. For example, the degradation of the bead can involve cleavage of a cleavable linkage via one or more species and/or methods described elsewhere herein. In another example, entrained species can be released from beads through osmotic pressure differences due to, for example, changing chemical environments. See, e.g., PCT/US2014/044398, which is hereby incorporated by reference in its entirety. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

A degradable bead can be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) can interact with other reagents contained in the partition. See, e.g., PCT/US2014/044398, which is hereby incorporated by reference in its entirety. For example, a polyacrylamide bead including cystamine and linked, via a disulfide bond, to a barcode sequence, can be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet including a bead-bound barcode sequence in basic solution can also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads can be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads can be accomplished by various swelling methods. The de-swelling of the beads can be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads can be accomplished by various de-swelling methods. Transferring the beads can cause pores in the bead to shrink. The shrinking can then hinder reagents within the beads from diffusing out of the interiors of the beads. The hindrance can be due to steric interactions between the reagents and the interiors of the beads. The transfer can be accomplished microfluidically. For instance, the transfer can be achieved by moving the beads from one co-flowing solvent stream to a different co-flowing solvent stream. The swellability and/or pore size of the beads can be adjusted by changing the polymer composition of the bead.

In some cases, an acrydite moiety linked to a precursor, another species linked to a precursor, or a precursor itself can include a labile bond, such as chemically, thermally, or photo-sensitive bond e.g., disulfide bond, UV sensitive bond, or the like. Once acrydite moieties or other moieties including a labile bond are incorporated into a bead, the bead can also include the labile bond. The labile bond can be, for example, useful in reversibly linking (e.g., covalently linking) species (e.g., barcodes, primers, etc.) to a bead. In some cases, a thermally labile bond can include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead.

The addition of multiple types of labile bonds to a gel bead can result in the generation of a bead capable of responding to varied stimuli. Each type of labile bond can be sensitive to an associated stimulus (e.g., chemical stimulus, light, temperature, enzymatic, etc.) such that release of species attached to a bead via each labile bond can be controlled by the application of the appropriate stimulus. Such functionality can be useful in controlled release of species from a gel bead. In some cases, another species including a labile bond can be linked to a gel bead after gel bead formation via, for example, an activated functional group of the gel bead as described above.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that can be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond can be cleavable via other nucleic acid molecule targeting enzymes, such as restriction enzymes (e.g., restriction endonucleases), as described further below.

Species can be encapsulated in beads (e.g., capture agent) during bead generation (e.g., during polymerization of precursors). Such species may or may not participate in polymerization. Such species can be entered into polymerization reaction mixtures such that generated beads include the species upon bead formation. In some cases, such species can be added to the gel beads after formation. Such species can include, for example, nucleic acid molecules (e.g., oligonucleotides), reagents for a nucleic acid amplification reaction (e.g., primers, polymerases, dNTPs, co-factors (e.g., ionic co-factors, buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species can include one or more enzymes described herein, including without limitation, polymerase, reverse transcriptase, restriction enzymes (e.g., endonuclease), transposase, ligase, proteinase K, DNAse, etc. Such species can include one or more reagents described elsewhere herein (e.g., lysis agents, inhibitors, inactivating agents, chelating agents, stimulus). Trapping of such species can be controlled by the polymer network density generated during polymerization of precursors, control of ionic charge within the gel bead (e.g., via ionic species linked to polymerized species), or by the release of other species. Encapsulated species can be released from a bead upon bead degradation and/or by application of a stimulus capable of releasing the species from the bead. Alternatively or in addition, species can be partitioned in a partition (e.g., droplet) during or subsequent to partition formation. Such species can include, without limitation, the abovementioned species that can also be encapsulated in a bead.

A degradable bead can include one or more species with a labile bond such that, when the bead/species is exposed to the appropriate stimuli, the bond is broken and the bead degrades. The labile bond can be a chemical bond (e.g., covalent bond, ionic bond) or can be another type of physical interaction (e.g., van der Waals interactions, dipole-dipole interactions, etc.). In some cases, a crosslinker used to generate a bead can include a labile bond. Upon exposure to the appropriate conditions, the labile bond can be broken and the bead degraded. For example, upon exposure of a polyacrylamide gel bead including cystamine crosslinkers to a reducing agent, the disulfide bonds of the cystamine can be broken and the bead degraded.

A degradable bead can be useful in more quickly releasing an attached species (e.g., a nucleic acid molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species can have greater mobility and accessibility to other species in solution upon degradation of the bead. In some cases, a species can also be attached to a degradable bead via a degradable linker (e.g., disulfide linker). The degradable linker can respond to the same stimuli as the degradable bead or the two degradable species can respond to different stimuli. For example, a barcode sequence can be attached, via a disulfide bond, to a polyacrylamide bead including cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation can refer to the disassociation of a bound or entrained species from a bead, both with and without structurally degrading the physical bead itself. For example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

Where degradable beads are provided, it can be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it can be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that can be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers can be used to trigger the degradation of beads. Examples of these chemical changes can include, but are not limited to pH-mediated changes to the integrity of a component within the bead, degradation of a component of a bead via cleavage of cross-linked bonds, and depolymerization of a component of a bead.

In some embodiments, a bead can be formed from materials that include degradable chemical crosslinkers, such as BAC or cystamine. Degradation of such degradable crosslinkers can be accomplished through a number of mechanisms. In some examples, a bead can be contacted with a chemical degrading agent that can induce oxidation, reduction or other chemical changes. For example, a chemical degrading agent can be a reducing agent, such as dithiothreitol (DTT). Additional examples of reducing agents can include 3-mercaptoethanol, (2S)-2-amino-1,4-dimercaptobutane (dithiobutylamine or DTBA), tris(2-carboxyethyl) phosphine (TCEP), or combinations thereof. A reducing agent can degrade the disulfide bonds formed between gel precursors forming the bead, and thus, degrade the bead. In other cases, a change in pH of a solution, such as an increase in pH, can trigger degradation of a bead. In other cases, exposure to an aqueous solution, such as water, can trigger hydrolytic degradation, and thus degradation of the bead. In some cases, any combination of stimuli can trigger degradation of a bead. For example, a change in pH can enable a chemical agent (e.g., DTT) to become an effective reducing agent.

Beads can also be induced to release their contents upon the application of a thermal stimulus. A change in temperature can cause a variety of changes to a bead. For example, heat can cause a solid bead to liquefy. A change in heat can cause melting of a bead such that a portion of the bead degrades. In other cases, heat can increase the internal pressure of the bead components such that the bead ruptures or explodes. Heat can also act upon heat-sensitive polymers used as materials to construct beads.

Any suitable agent can degrade beads. In some embodiments, changes in temperature or pH can be used to degrade thermo-sensitive or pH-sensitive bonds within beads. In some embodiments, chemical degrading agents can be used to degrade chemical bonds within beads by oxidation, reduction or other chemical changes. For example, a chemical degrading agent can be a reducing agent, such as DTT, wherein DTT can degrade the disulfide bonds formed between a crosslinker and gel precursors, thus degrading the bead. In some embodiments, a reducing agent can be added to degrade the bead, which may or may not cause the bead to release its contents. Examples of reducing agents can include dithiothreitol (DTT), (3-mercaptoethanol, (2S)-2-amino-1,4-dimercaptobutane (dithiobutylamine or DTBA), tris(2-carboxyethyl) phosphine (TCEP), or combinations thereof. The reducing agent can be present at a concentration of about 0.1 mM, 0.5 mM, 1 mM, 5 mM, or 10 mM. The reducing agent can be present at a concentration of at least about 0.1 mM, 0.5 mM, 1 mM, 5 mM, 10 mM, or greater than 10 mM. The reducing agent can be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing oligonucleotide bearing beads.

Although FIG. 12 and FIG. 13 have been described in terms of providing substantially singly occupied partitions, above, in certain cases, it may be desirable to provide multiply occupied partitions, e.g., containing two, three, four or more cells, nuclei and/or beads including nucleic acid barcode molecules (e.g., oligonucleotides) within a single partition (e.g., multi-omics method described elsewhere, herein). Accordingly, as noted above, the flow characteristics of the biological particle and/or bead containing fluids and partitioning fluids can be controlled to provide for such multiply occupied partitions. In particular, the flow parameters can be controlled to provide a given occupancy rate at greater than about 50% of the partitions, greater than about 75%, and in some cases greater than about 80%, 90%, 95%, or higher.

In some cases, additional beads can be used to deliver additional reagents to a partition. In such cases, it can be advantageous to introduce different beads into a common channel or droplet generation junction, from different bead sources (e.g., containing different associated reagents) through different channel inlets into such common channel or droplet generation junction (e.g., junction 1210). In such cases, the flow and frequency of the different beads into the channel or junction can be controlled to provide for a certain ratio of beads from each source, while ensuring a given pairing or combination of such beads into a partition with a given number of biological particles (e.g., one biological particle and one bead per partition).

The partitions described herein can include small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets can have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions can be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species can generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions can include both unoccupied partitions (e.g., empty partitions) and occupied partitions.

### Reagents

In accordance with certain aspects, biological particles can be partitioned along with lysis reagents in order to release the contents of the biological particles within the partition. See, e.g., U.S. Pat. Pub. 2018/0216162 (now U.S. Pat. No. 10,428,326), U.S. Pat. Pub. 2019/0100632 (now U.S. Pat. No. 10,590,244), and U.S. Pat. Pub. 2019/0233878. Biological particles (e.g., cells, nuclei, cell beads, cell nuclei, organelles, and the like) can be partitioned together with nucleic acid barcode molecules and the nucleic acid molecules of or derived from the biological particle (e.g., mRNA, cDNA, gDNA, etc.,) can be barcoded as described elsewhere herein. In some embodiments, biological particles are co-partitioned with barcode carrying beads (e.g., gel beads) and the nucleic acid molecules of or derived from the biological particle are barcoded as described elsewhere herein. In such cases, the lysis agents can be contacted with the biological particle suspension concurrently with, or immediately prior to, the introduction of the biological particles into the partitioning junction/droplet generation zone (e.g., junction 1210), such as through an additional channel or channels upstream of the channel junction. In accordance with other aspects, additionally or alternatively, biological particles can be partitioned along with other reagents, as will be described further below.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments described herein can be coupled to any of a variety of different fluid sources or receiving components, including reservoirs, tubing, manifolds, or fluidic components of other systems. As will be appreciated, the microfluidic channel structures can have other geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents can additionally or alternatively be co-partitioned with the biological particles to cause the release of the biological particle's contents into the partitions. For example, in some cases, surfactant-based lysis solutions can be used to lyse nuclei or cells (e.g., labelled engineered cells), although these can be less desirable for emulsion based systems where the surfactants can interfere with stable emulsions. In some cases, lysis solutions can include non-ionic surfactants such as, for example, Triton X-100 and Tween 20. In some cases, lysis solutions can include ionic surfactants such as, for example, sarcosyl and sodium dodecyl sulfate (SDS). Electroporation, thermal, acoustic or mechanical cellular disruption can also be used in certain cases, e.g., non-emulsion based partitioning such as encapsulation of biological particles that can be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles (e.g., labelled nuclei or engineered cells) described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., cell beads comprising labelled cells), the biological particles can be exposed to an appropriate stimulus to release the biological particles or their contents from a co-partitioned cell bead. For example, in some cases, a chemical stimulus can be co-partitioned along with an encapsulated biological particle to allow for the degradation of the encapsulating material and release of the cell, nucleus, or its contents into the larger partition. In some cases, this stimulus can be the same as the stimulus described elsewhere herein for release of nucleic acid molecules (e.g., oligonucleotides) from their respective bead. In alternative aspects, this can be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents can also be co-partitioned with the biological particles (e.g., labelled nuclei or cells, e.g., immune or tumor cells), such as endonucleases to fragment a biological particle's DNA, DNA polymerase enzymes and dNTPs used to amplify the biological particle's nucleic acid fragments and to attach the barcode molecular tags to the amplified fragments. Other enzymes can be co-partitioned, including without limitation, polymerase, transposase, ligase, proteinase K, DNAse, etc. Additional reagents can also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides can include a hybridization region and a template region. The hybridization region can include any sequence capable of hybridizing to the target. In some cases, as previously described, the hybridization region includes a series of G bases to complement the overhanging C bases at the 3′ end of a cDNA molecule. The series of G bases can include 1 G base, 2 G bases, 3 G bases, 4 G bases, 5 G bases or more than 5 G bases. The template sequence can include any sequence to be incorporated into the cDNA. In some cases, the template region includes at least 1 (e.g., at least 2, 3, 4, 5 or more) tag sequences and/or functional sequences. Switch oligos can include deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo can be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the nuclei or cells (e.g., immune or tumor cells) are released into their respective partitions, the macromolecular components (e.g., macromolecular constituents of biological particles, such as RNA, DNA, proteins, or secreted analytes) contained therein can be further processed within the partitions. In accordance with the methods and systems described herein, the macromolecular component contents of individual biological particles (e.g., nuclei or cells, such as immune or tumor cells) can be provided with unique identifiers such that, upon characterization of those macromolecular components they can be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle (e.g., nuclei or cells, such as immune or tumor cells) or groups of biological particles (e.g., nuclei or tumor cells) with the unique identifiers, such as described above (with reference to FIGS. 12 and 13). In some aspects, the unique identifiers are provided in the form of nucleic acid molecules (e.g., oligonucleotides) that include nucleic acid barcode sequences that can be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences can be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence can be about 6, 7, 8, 9, 10, 11, 12, 13, 14, 15, 16, 17, 18, 19, 20 nucleotides or longer. In some cases, the length of a barcode sequence can be at least about 6, 7, 8, 9, 10, 11, 12, 13, 14, 15, 16, 17, 18, 19, 20 nucleotides or longer. In some cases, the length of a barcode sequence can be at most about 6, 7, 8, 9, 10, 11, 12, 13, 14, 15, 16, 17, 18, 19, 20 nucleotides or shorter. These nucleotides can be completely contiguous, i.e., in a single stretch of adjacent nucleotides, or they can be separated into two or more separate subsequences that are separated by 1 or more nucleotides. In some cases, separated barcode subsequences can be from about 4 to about 16 nucleotides in length. In some cases, the barcode subsequence can be about 4, 5, 6, 7, 8, 9, 10, 11, 12, 13, 14, 15, 16 nucleotides or longer. In some cases, the barcode subsequence can be at least about 4, 5, 6, 7, 8, 9, 10, 11, 12, 13, 14, 15, 16 nucleotides or longer. In some cases, the barcode subsequence can be at most about 4, 5, 6, 7, 8, 9, 10, 11, 12, 13, 14, 15, 16 nucleotides or shorter.

The co-partitioned nucleic acid molecules can also include other functional sequences useful in the processing of the nucleic acids from the co-partitioned biological particles (e.g., labelled nuclei or cells, e.g., immune or tumor cells). These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides can also be employed, including, e.g., coalescence of two or more droplets, where one droplet contains oligonucleotides, or microdispensing of oligonucleotides into partitions, e.g., droplets within microfluidic systems.

In an example, beads are provided that each include large numbers of the above described nucleic acid barcode molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., including polyacrylamide polymer matrices, are used as a solid support and delivery vehicle for the nucleic acid molecules into the partitions, as they are capable of carrying large numbers of nucleic acid molecules, and can be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. In some embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set. In some embodiments, such different barcode sequences can be associated with a given bead.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences can provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus can be a photo-stimulus, e.g., through cleavage of a photo-labile linkage that releases the nucleic acid molecules. In other cases, a thermal stimulus can be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and can be degraded for release of the attached nucleic acid molecules through exposure to a reducing agent, such as DTT.

### Systems and Methods for Controlled Partitioning

In some aspects, provided are systems and methods for controlled partitioning.

Droplet size can be controlled by adjusting certain geometric features in channel architecture (e.g., microfluidics channel architecture). For example, an expansion angle, width, and/or length of a channel can be adjusted to control droplet size.

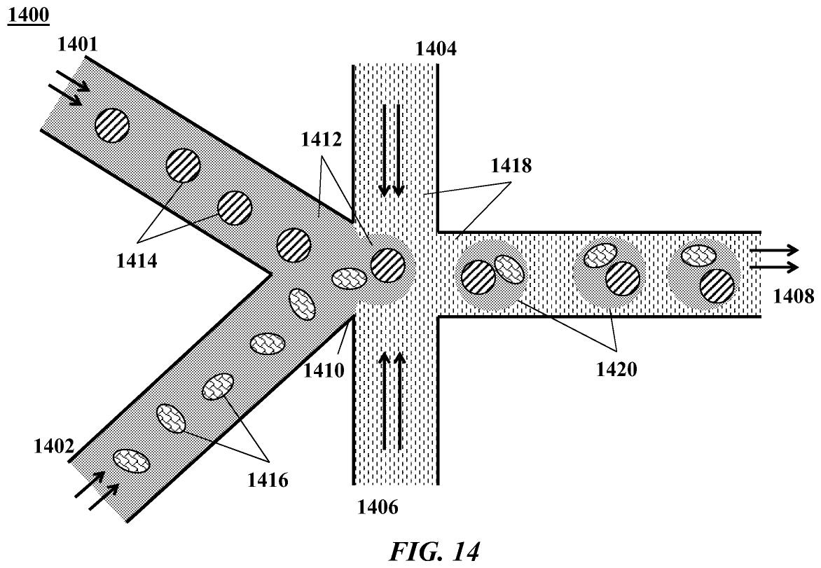

FIG. 14 shows an example of a microfluidic channel structure 1400 for delivering barcode carrying beads to droplets. The channel structure 1400 can include channel segments 1401, 1416, 1404, 1406 and 1408 communicating at a channel junction 1410. In operation, the channel segment 1401 may transport an aqueous fluid 1412 that includes a plurality of beads 1414 (e.g., with nucleic acid molecules, oligonucleotides, molecular tags) along the channel segment 1401 into junction 1410. The plurality of beads 1414 may be sourced from a suspension of beads. For example, the channel segment 1401 may be connected to a reservoir comprising an aqueous suspension of beads 1414. The channel segment 1402 may transport the aqueous fluid 1412 that includes a plurality of biological particles 1416 along the channel segment 1402 into junction 1410. The plurality of biological particles 1416 may be sourced from a suspension of biological particles. For example, the channel segment 1402 may be connected to a reservoir comprising an aqueous suspension of biological particles 1416. In some instances, the aqueous fluid 1412 in either the first channel segment 1401 or the second channel segment 1402, or in both segments, can include one or more reagents, as further described below. A second fluid 1418 that is immiscible with the aqueous fluid 1412 (e.g., oil) can be delivered to the junction 1410 from each of channel segments 1404 and 1406. Upon meeting of the aqueous fluid 1412 from each of channel segments 1401 and 1402 and the second fluid 1418 from each of channel segments 1404 and 1406 at the channel junction 1410, the aqueous fluid 1412 can be partitioned as discrete droplets 1420 in the second fluid 1418 and flow away from the junction 1410 along channel segment 1408. The channel segment 1408 may deliver the discrete droplets to an outlet reservoir fluidly coupled to the channel segment 1408, where they may be harvested. As an alternative, the channel segments 1401 and 1402 may meet at another junction upstream of the junction 1410. At such junction, beads and biological particles may form a mixture that is directed along another channel to the junction 1410 to yield droplets 1420. The mixture may provide the beads and biological particles in an alternating fashion, such that, for example, a droplet comprises a single bead and a single biological particle.

FIG. 13 shows an example of a microfluidic channel structure for the controlled partitioning of beads into discrete droplets. A channel structure 1300 can include a channel segment 1302 communicating at a channel junction 1306 (or intersection) with a reservoir 1304. The reservoir 1304 can be a chamber. Any reference to “reservoir,” as used herein, can also refer to a “chamber.” In operation, an aqueous fluid 1308 that includes suspended beads 1312 can be transported along the channel segment 1302 into the junction 1306 to meet a second fluid 1310 that is immiscible with the aqueous fluid 1308 in the reservoir 1304 to create droplets 1316, 1318 of the aqueous fluid 1308 flowing into the reservoir 1304. At the junction 1306 where the aqueous fluid 1308 and the second fluid 1310 meet, droplets can form based on factors such as the hydrodynamic forces at the junction 1306, flow rates of the two fluids 1308, 1310, fluid properties, and certain geometric parameters (e.g., w, ho, a, etc.) of the channel structure 1300. A plurality of droplets can be collected in the reservoir 1304 by continuously injecting the aqueous fluid 1308 from the channel segment 1302 through the junction 1306.

A discrete droplet generated can include a bead (e.g., as in occupied droplets 1316). Alternatively, a discrete droplet generated can include more than one bead. Alternatively, a discrete droplet generated cannot include any beads (e.g., as in unoccupied droplet 1318). In some instances, a discrete droplet generated can contain one or more biological particles, as described elsewhere herein. In some instances, a discrete droplet generated can include one or more reagents, as described elsewhere herein.

In some instances, the aqueous fluid 1308 can have a substantially uniform concentration or frequency of beads 1312. The beads 1312 can be introduced into the channel segment 1302 from a separate channel (not shown in FIG. 13). The frequency of beads 1312 in the channel segment 1302 can be controlled by controlling the frequency in which the beads 1312 are introduced into the channel segment 1302 and/or the relative flow rates of the fluids in the channel segment 1302 and the separate channel. In some instances, the beads can be introduced into the channel segment 1302 from a plurality of different channels, and the frequency controlled accordingly.

In some instances, the aqueous fluid 1308 in the channel segment 1302 can include biological particles (e.g., described with reference to FIG. 12). In some instances, the aqueous fluid 1308 can have a substantially uniform concentration or frequency of biological particles. As with the beads, the biological particles (e.g., labelled nuclei or cells) can be introduced into the channel segment 1302 from a separate channel. The frequency or concentration of the biological particles in the aqueous fluid 1308 in the channel segment 1302 can be controlled by controlling the frequency in which the biological particles are introduced into the channel segment 1302 and/or the relative flow rates of the fluids in the channel segment 1302 and the separate channel. In some instances, the biological particles can be introduced into the channel segment 1302 from a plurality of different channels, and the frequency controlled accordingly. In some instances, a first separate channel can introduce beads and a second separate channel can introduce biological particles into the channel segment 1302. The first separate channel introducing the beads can be upstream or downstream of the second separate channel introducing the biological particles.

The second fluid 1310 can include an oil, such as a fluorinated oil, that includes a fluorosurfactant for stabilizing the resulting droplets, for example, inhibiting subsequent coalescence of the resulting droplets.

In some instances, the second fluid 1310 cannot be subjected to and/or directed to any flow in or out of the reservoir 1304. For example, the second fluid 1310 can be substantially stationary in the reservoir 1304. In some instances, the second fluid 1310 can be subjected to flow within the reservoir 1304, but not in or out of the reservoir 1304, such as via application of pressure to the reservoir 1304 and/or as affected by the incoming flow of the aqueous fluid 1308 at the junction 1306. Alternatively, the second fluid 1310 can be subjected and/or directed to flow in or out of the reservoir 1304. For example, the reservoir 1304 can be a channel directing the second fluid 1310 from upstream to downstream, transporting the generated droplets.

Systems and methods for controlled partitioning are described further in PCT/US2018/047551, which is hereby incorporated by reference in its entirety.

The channel structure 1300 at or near the junction 1306 can have certain geometric features that at least partly determine the sizes of the droplets formed by the channel structure 1300. The channel segment 1302 can have a height, ho and width, w, at or near the junction 1306. By way of example, the channel segment 1302 can include a rectangular cross-section that leads to a reservoir 1304 having a wider cross-section (such as in width or diameter). Alternatively, the cross-section of the channel segment 1302 can be other shapes, such as a circular shape, trapezoidal shape, polygonal shape, or any other shapes. The top and bottom walls of the reservoir 1304 at or near the junction 1306 can be inclined at an expansion angle, a. The expansion angle, a, allows the tongue (portion of the aqueous fluid 1308 leaving channel segment 1302 at junction 1306 and entering the reservoir 1304 before droplet formation) to increase in depth and facilitate decrease in curvature of the intermediately formed droplet.

Droplet size can decrease with increasing expansion angle. The resulting droplet radius, Rd, can be predicted by the following equation for the aforementioned geometric parameters of ho, and a:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and a=3°, the predicted droplet size is 121 μm. In another example, for a channel structure with w=25 μm, h=25 μm, and a=5°, the predicted droplet size is 123 μm. In another example, for a channel structure with w=28 μm, h=28 μm, and a=7°, the predicted droplet size is 124 μm.

In some instances, the expansion angle, a, can be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 1308 entering the junction 1306 can be between about 0.04 microliters (μL)/minute (min) and about 40 μL/min. In some instances, the flow rate of the aqueous fluid 1308 entering the junction 1306 can be between about 0.01 microliters (μL)/minute (min) and about 100 μL/min. Alternatively, the flow rate of the aqueous fluid 1308 entering the junction 1306 can be less than about 0.01 μL/min. Alternatively, the flow rate of the aqueous fluid 1308 entering the junction 1306 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius cannot be dependent on the flow rate of the aqueous fluid 1308 entering the junction 1306.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 1306) between aqueous fluid 1308 channel segments (e.g., channel segment 1302) and the reservoir 1304. Alternatively or in addition, the throughput of droplet generation can be increased by increasing the flow rate of the aqueous fluid 1308 in the channel segment 1302.

The methods and systems described herein can be used to greatly increase the efficiency of single cell or nucleus applications and/or other applications receiving droplet-based input.

Subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. Additional reagents that can be co-partitioned along with the barcode bearing bead can include oligonucleotides to block ribosomal RNA (rRNA) and nucleases to digest genomic DNA from cells or nuclei. Alternatively, rRNA removal agents can be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, can be subject to sequencing for sequence analysis. In some cases, amplification can be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Partitions including a barcode bead (e.g., a gel bead) associated with barcode molecules and a bead encapsulating cellular constituents (e.g., a cell bead) such as cellular nucleic acids can be useful in constituent analysis as is described in U.S. Patent Publication No. 2018/0216162.

### Sample and Cell Processing

A sample can be derived from any useful source including any subject, such as a human subject. A sample can include material (e.g., one or more cells or nuclei) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apart)), or multiple samples from different subjects, can be obtained for analysis as described herein. For example, a first sample can be obtained from a subject at a first time and a second sample can be obtained from the subject at a second time later than the first time. The first time can be before a subject undergoes a treatment regimen or procedure (e.g., to address a disease or condition), and the second time can be during or after the subject undergoes the treatment regimen or procedure. In another example, a first sample can be obtained from a first bodily location or system of a subject (e.g., using a first collection technique) and a second sample can be obtained from a second bodily location or system of the subject (e.g., using a second collection technique), which second bodily location or system can be different than the first bodily location or system. In another example, multiple samples can be obtained from a subject at a same time from the same or different bodily locations. Different samples, such as different samples collected from different bodily locations of a same subject, at different times, from multiple different subjects, and/or using different collection techniques, can undergo the same or different processing (e.g., as described herein). For example, a first sample can undergo a first processing protocol and a second sample can undergo a second processing protocol.

A sample can be a biological sample, such as a cell or nucleus sample (e.g., as described herein). A sample can include one or more biological particles, such as one or more cells and/or cellular constituents, such as one or more cell nuclei. For example, a sample can include a plurality of cells or nuclei and/or cellular constituents. Components (e.g., cells or cellular constituents, such as cell nuclei) of a sample can be of a single type or a plurality of different types. For example, cells of a sample can include one or more different types of blood cells.

Cells and cellular constituents of a sample may be of any type. For example, a cell or cellular constituent may be a vertebral, mammalian, fungal, plant, bacterial, or other cell type. In some cases, the cell is a mammalian cell, such as a human cell. The cell may be, for example, a stem cell, liver cell, nerve cell, bone cell, blood cell, reproductive cell, skin cell, skeletal muscle cell, cardiac muscle cell, smooth muscle cell, hair cell, hormone-secreting cell, or glandular cell. The cell may be, for example, an erythrocyte (e.g., red blood cell), a megakaryocyte (e.g., platelet precursor), a monocyte (e.g., white blood cell), a leukocyte, a B cell, a T cell (such as a helper, suppressor, cytotoxic, or natural killer T cell), an osteoclast, a dendritic cell, a connective tissue macrophage, an epidermal Langerhans cell, a microglial cell, a granulocyte, a hybridoma cell, a mast cell, a natural killer cell, a reticulocyte, a hematopoietic stem cell, a myoepithelial cell, a myeloid-derived suppressor cell, a platelet, a thymocyte, a satellite cell, an epithelial cell, an endothelial cell, an epididymal cell, a kidney cell, a liver cell, an adipocyte, a lipocyte, or a neuron cell. In some cases, the cell may be associated with a cancer, tumor, or neoplasm. In some cases, the cell may be associated with a fetus. In some cases, the cell may be a Jurkat cell.

A biological sample can include a plurality of cells or nuclei having different dimensions and features. In some cases, processing of the biological sample, such as cell separation and sorting (e.g., as described herein), can affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process can include the use of microfluidics (e.g., to separate biological particles of different sizes, types, charges, or other features).

In an example, a sample including one or more cells or nuclei can be processed to separate the one or more cells or nuclei from other materials in the sample (e.g., using centrifugation and/or another process). In some cases, cells and/or cellular constituents, e.g., nuclei, of a sample can be processed to separate and/or sort groups of cells and/or cellular constituents, such as to separate and/or sort cells and/or cellular constituents of different types.

Examples of cell separation include, but are not limited to, separation of white blood cells or immune cells from other blood cells and components, separation of circulating tumor cells from blood, and separation of bacteria from bodily cells and/or environmental materials. A separation process can include a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells or nuclei can include, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method can be used to detect cells and/or cellular constituents based on a parameter such as a size, morphology, or protein expression. Flow cytometry-based cell sorting can include injecting a sample into a sheath fluid that conveys the cells and/or cellular constituents of the sample into a measurement region one at a time. In the measurement region, a light source such as a laser can interrogate the cells and/or cellular constituents and scattered light and/or fluorescence can be detected and converted into digital signals. A nozzle system (e.g., a vibrating nozzle system) can be used to generate droplets (e.g., aqueous droplets) including individual cells and/or cellular constituents. Droplets including cells and/or cellular constituents of interest (e.g., as determined via optical detection) can be labeled with an electric charge (e.g., using an electrical charging ring), which charge can be used to separate such droplets from droplets including other cells and/or cellular constituents. For example, FACS can include labeling cells and/or cellular constituents with fluorescent markers (e.g., using internal and/or external biomarkers). Cells, nuclei, and/or cellular constituents can then be measured and identified one by one and sorted based on the emitted fluorescence of the marker or absence thereof. MACS can use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS can use microbubbles (e.g., glass microbubbles) labeled with antibodies to target cells of interest. Cells and/or cellular components coupled to microbubbles can float to a surface of a solution, thereby separating target cells and/or cellular components from other components of a sample. Cell separation techniques can be used to enrich for populations of cells of interest (e.g., prior to partitioning, as described herein). For example, a sample including a plurality of cells including a plurality of cells of a given type can be subjected to a positive separation process. The plurality of cells of the given type can be labeled with a fluorescent marker (e.g., based on an expressed cell surface marker or another marker) and subjected to a FACS process to separate these cells from other cells of the plurality of cells. The selected cells can then be subjected to subsequent partition-based analysis (e.g., as described herein) or other downstream analysis. The fluorescent marker can be removed prior to such analysis or can be retained. The fluorescent marker can include an identifying feature, such as a nucleic acid barcode sequence and/or unique molecular identifier.

In another example, a first sample including a first plurality of cells including a first plurality of cells of a given type (e.g., immune cells expressing a particular marker or combination of markers) and a second sample including a second plurality of cells including a second plurality of cells of the given type can be subjected to a positive separation process. The first and second samples can be collected from the same or different subjects, at the same or different types, from the same or different bodily locations or systems, using the same or different collection techniques. For example, the first sample can be from a first subject and the second sample can be from a second subject different than the first subject. The first plurality of cells of the first sample can be provided a first plurality of fluorescent markers configured to label the first plurality of cells of the given type. The second plurality of cells of the second sample can be provided a second plurality of fluorescent markers configured to label the second plurality of cells of the given type. The first plurality of fluorescent markers can include a first identifying feature, such as a first barcode, while the second plurality of fluorescent markers can include a second identifying feature, such as a second barcode, that is different than the first identifying feature. The first plurality of fluorescent markers and the second plurality of fluorescent markers can fluoresce at the same intensities and over the same range of wavelengths upon excitation with a same excitation source (e.g., light source, such as a laser). The first and second samples can then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples can undergo separate FACS processes and the positively selected cells of the given type from the first sample and the positively selected cells of the given type from the second sample can then be combined for subsequent analysis. The encoded identifying features of the different fluorescent markers can be used to identify cells originating from the first sample and cells originating from the second sample. For example, the first and second identifying features can be configured to interact (e.g., in partitions, as described herein) with nucleic acid barcode molecules (e.g., as described herein) to generate barcoded nucleic acid products detectable using, e.g., nucleic acid sequencing.

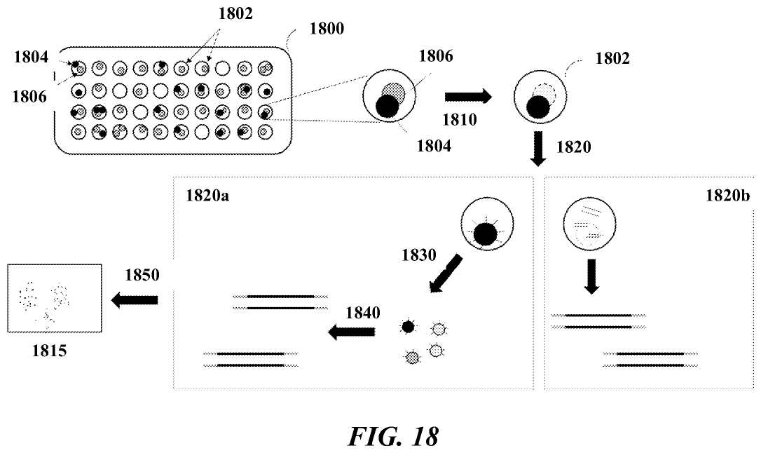

FIG. 18 schematically shows an example workflow for processing nucleic acid molecules within a sample. A substrate 1800 including a plurality of microwells 1802 can be provided. A sample 1806 which can include a cell, nucleus, cell bead, cellular components or analytes (e.g., proteins and/or nucleic acid molecules) can be co-partitioned, in a plurality of microwells 1802, with a plurality of beads 1804 including nucleic acid barcode molecules. During a partitioning process, the sample 1806 can be processed within the partition. For instance, in the case of live cells, the cell can be subjected to conditions sufficient to lyse the cells and release the analytes contained therein. In process 1820, the bead 1804 can be further processed. By way of example, processes 1820a and 1820b schematically illustrate different workflows, depending on the properties of the bead 1804.

In 1820a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. Such beads may be solid beads or magnetic beads to allow for removal from the microwell array. In process 1830, the beads 1804 from multiple wells 1802 can be collected and pooled. Further processing can be performed in process 1840. For example, one or more nucleic acid reactions can be performed, such as reverse transcription, nucleic acid extension, amplification, ligation, transposition, etc. In some embodiments, one or more reactions occur on the bead using the sample nucleic acid molecules captured on the bead via the nucleic acid barcode molecules on the bead, e.g., sample nucleic acid molecules hybridized to complementary sequences of the nucleic acid barcode molecules on the bead. In some instances, adapter sequences are ligated to the nucleic acid molecules, or derivatives thereof, as described elsewhere herein. For instance, sequencing primer sequences can be appended to each end of the nucleic acid molecule. In process 1850, further characterization, such as sequencing can be performed to generate sequencing reads. The sequencing reads can yield information on individual cells or populations of cells, which can be represented visually or graphically, e.g., in a plot.

In 1820b, the bead includes nucleic acid barcode molecules that are releasably attached thereto, as described below. The bead can degrade or otherwise release the nucleic acid barcode molecules into the well 1802; the nucleic acid barcode molecules can then be used to barcode nucleic acid molecules within the well 1802. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 1850, further characterization, such as sequencing can be performed to generate sequencing reads. The sequencing reads can yield information on individual cells or populations of cells, which can be represented visually or graphically, e.g., in a plot.

### Fixed Samples

A sample may be a fixed sample. For example, a sample may comprise a plurality of fixed samples, such as a plurality of fixed cells or fixed nuclei. Alternatively or in addition, a sample may comprise a fixed tissue. Fixation of cell or cellular constituent, or a tissue comprising a plurality of cells or nuclei, may comprise application of a chemical species or chemical stimulus. The term “fixed” as used herein with regard to biological samples generally refers to the state of being preserved from decay and/or degradation. “Fixation” generally refers to a process that results in a fixed sample, and in some instances can include contacting the biomolecules within a biological sample with a fixative (or fixation reagent) for some amount of time, whereby the fixative results in covalent bonding interactions such as crosslinks between biomolecules in the sample. A “fixed biological sample” may generally refer to a biological sample that has been contacted with a fixation reagent or fixative. For example, a formaldehyde-fixed biological sample has been contacted with the fixation reagent formaldehyde. “Fixed cells” or “fixed tissues” generally refer to cells or tissues that have been in contact with a fixative under conditions sufficient to allow or result in the formation of intra- and inter-molecular covalent crosslinks between biomolecules in the biological sample. Generally, contact of biological sample (e.g., a cell or nucleus) with a fixation reagent (e.g., paraformaldehyde or PFA) results in the formation of intra- and inter-molecular covalent crosslinks between biomolecules in the biological sample. In some cases, provision of the fixation reagent, such as formaldehyde, may result in covalent aminal crosslinks within RNA, DNA, and/or protein molecules. For example, the widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample. Examples of fixation reagents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like.

Other examples of fixation reagents include, for example, organic solvents such as alcohols (e.g., methanol or ethanol), ketones (e.g., acetone), and aldehydes (e.g., paraformaldehyde, formaldehyde (e.g., formalin), or glutaraldehyde). As described herein, cross-linking agents may also be used for fixation including, without limitation, disuccinimidyl suberate (DSS), dimethylsuberimidate (DMS), formalin, and dimethyladipimidate (DMA), dithio-bis(-succinimidyl propionate) (DSP), disuccinimidyl tartrate (DST), and ethylene glycol bis(succinimidyl succinate) (EGS). In some cases, a cross-linking agent may be a cleavable cross-linking agent (e.g., thermally cleavable, photocleavable, etc.). In some cases, more than one fixation reagent can be used in combination when preparing a fixed biological sample. Changes to a characteristic or a set of characteristics of a cell or cellular constituents (e.g., incurred upon interaction with one or more fixation agents) may be at least partially reversible (e.g., via rehydration or de-crosslinking). Alternatively, changes to a characteristic or set of characteristics of a cell or cellular constituents (e.g., incurred upon interaction with one or more fixation agents) may be substantially irreversible.

### Multiplexing Methods

In some embodiments of the disclosure, steps (a) and (b) of the methods described herein are performed in multiplex format. For example, in some embodiments, step (a) of the methods disclosed herein can include providing additional individual partitions of single nuclei or cells (e.g., immune or tumor cells), and step (b) can further include generating barcoded nucleic acid molecules produced in each of the additional individual partitions of nuclei or cells (e.g., immune or tumor cells).

Accordingly, in some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more nuclei, cells or cell features can be used to characterize nuclei, cells and/or cell features. In some instances, cell features include cell surface features. Cell surface features can include, but are not limited to, a receptor, an antigen or antigen fragment (e.g., an antigen or antigen fragment that binds to an antigen-binding molecule located on a cell surface), a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. A labelling agent can include, but is not limited to, a protein, a peptide, an antibody (or an epitope binding fragment thereof), an antigen, an antigen fragment, a lipophilic moiety (such as cholesterol), a cell surface receptor binding molecule, a receptor ligand, a small molecule, a B-cell receptor engager, a pro-body, an aptamer, a monobody, an affimer, a Darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell feature (e.g., a first cell surface feature) can have a first reporter oligonucleotide coupled thereto, while a labelling agent that is specific to a different cell feature (e.g., a second cell surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969.

In a particular example, a library of potential cell feature labelling agents can be provided, where the respective cell feature labelling agents are associated with nucleic acid reporter molecules, such that a different reporter oligonucleotide sequence is associated with each labelling agent capable of binding to a specific cell feature. In some embodiments, the cell feature labelling agents comprise a reporter agent comprising an aptamer specific for a secreted analyte, as disclosed herein. In some embodiments, the cell feature labelling agents comprise a plurality of reporter agents, each comprising an aptamer specific for a different secreted analyte. In other aspects, different members of the plurality can be characterized by the presence of a different oligonucleotide sequence label.

Labelling agents capable of binding to or otherwise coupling to one or more cells or nuclei can be used to characterize a cell or nucleus as belonging to a particular set. For example, labelling agents can be used to label a sample of cells or nuclei, e.g., to provide a sample index. For other example, labelling agents can be used to label a group of cells or nuclei belonging to a particular experimental condition. In this way, a group of cells or nuclei can be labeled as different from another group of cells or nuclei. In an example, a first group of cells or nuclei can originate from a first sample and a second group of cells or nuclei can originate from a second sample. Labelling agents can allow the first group and second group to have a different labeling agent (or reporter oligonucleotide associated with the labeling agent). This can, for example, facilitate multiplexing, where cells or nuclei of the first group and cells or nuclei of the second group can be labeled separately and then pooled together for downstream analysis. The downstream detection of a label can indicate analytes as belonging to a particular group.

For example, a reporter oligonucleotide may be linked to an antibody or an epitope binding fragment thereof, and labeling a cell or nuclei may comprise subjecting the antibody-linked barcode molecule or the epitope binding fragment-linked barcode molecule to conditions suitable for binding the antibody to a molecule present on a surface of the cell or nucleus for detection of cell surface proteins or nuclear membrane proteins. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant can be less than about 10 μM. In some embodiments, the secreted analyte has a desired off rate (k_{off}), such that the secreted analyte remains coupled to the reporter agent during various sample processing steps.

In another example, a reporter oligonucleotide can be coupled to a cell-penetrating peptide (CPP), and labeling cells can include delivering the CPP coupled to the reporter oligonucleotide into a biological particle. Labeling biological particles can include delivering the CPP conjugated, e.g., coupled to the, oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649 for a description of organic fluorophores.

A reporter oligonucleotide can be coupled to a lipophilic molecule, and labeling cells or nuclei can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the cells or nuclei can be incubated with the library of labelling agents, that can be labelling agents to a broad panel of different cell or nucleus features, e.g., receptors, proteins, etc., and which include their associated reporter oligonucleotides. Unbound labelling agents can be washed from the cells or nuclei, and the cells or nuclei can then be co-partitioned (e.g., into droplets or wells) along with partition-specific barcode oligonucleotides (e.g., attached to a support, such as a bead or gel bead) as described elsewhere herein. As a result, the partitions can include the cell or cells (or nucleus or nuclei), as well as the bound labelling agents and their known, associated reporter oligonucleotides.

In other instances, e.g., to facilitate sample multiplexing, a labelling agent that is specific to a particular cell or nucleus feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent can interact with different cells or nuclei, cell or nucleus populations or samples, allowing a particular report oligonucleotide to indicate a particular cell or nucleus population (or cell or nucleus or sample) and cell/nucleus feature. In this way, different samples or groups can be independently processed and subsequently combined together for pooled analysis (e.g., partition-based barcoding as described elsewhere herein). See, e.g., U.S. Pat. Pub. 20190323088.

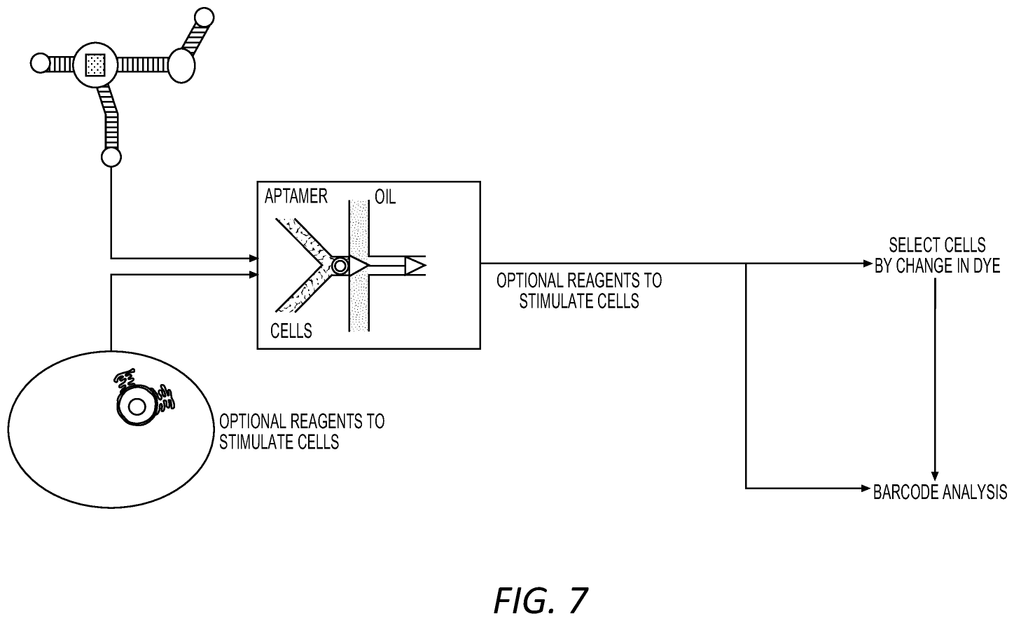

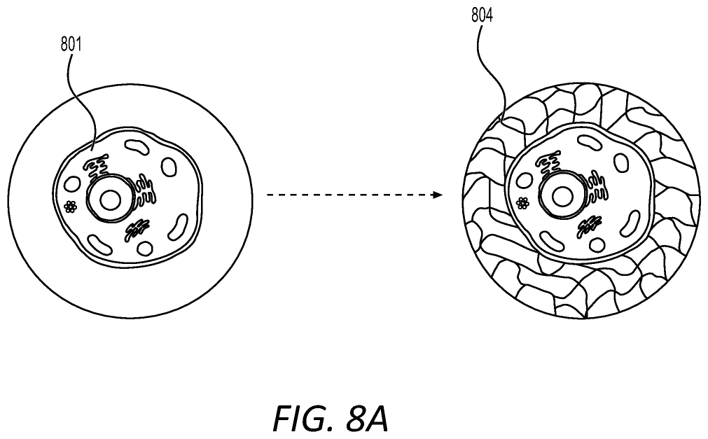

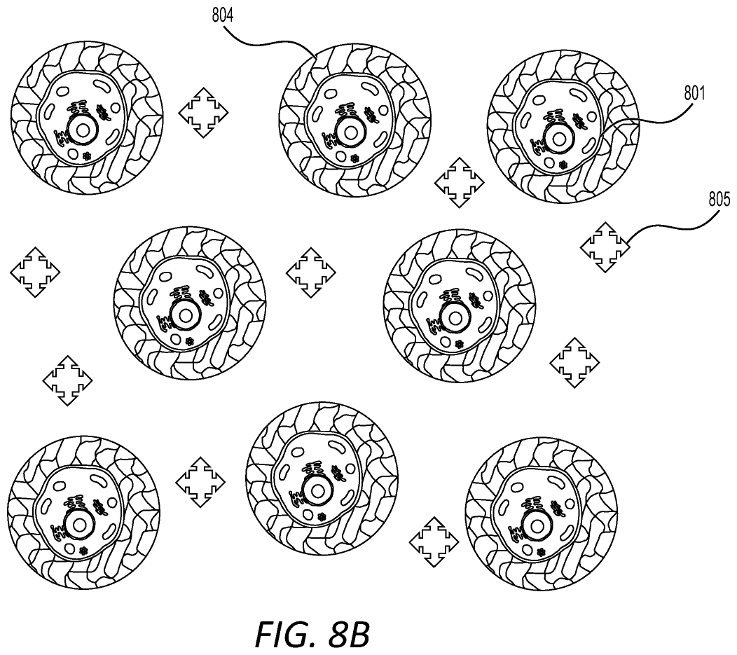

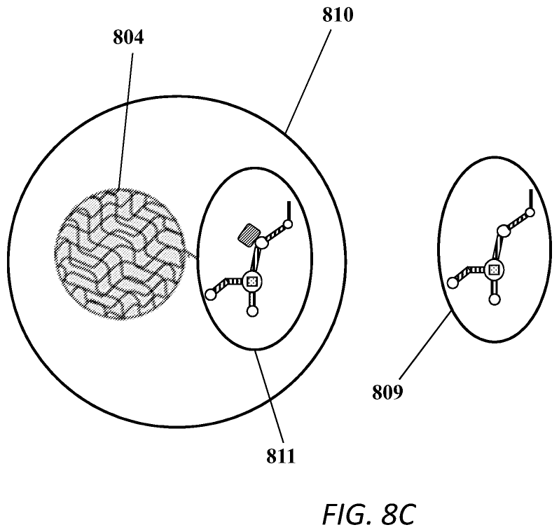

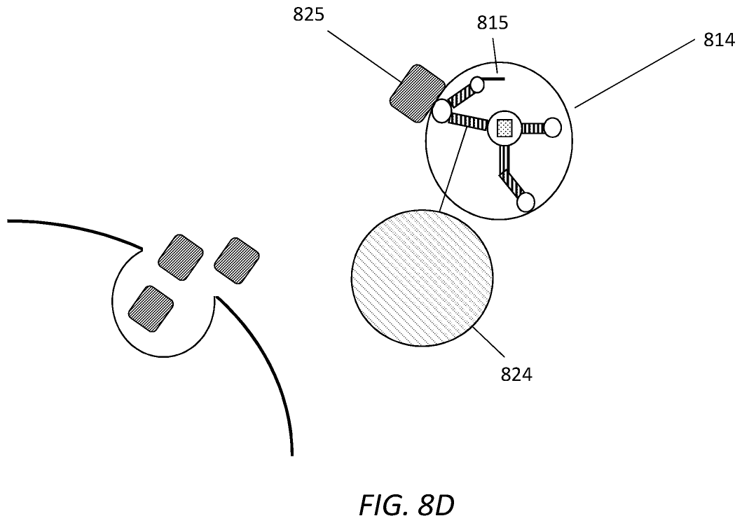

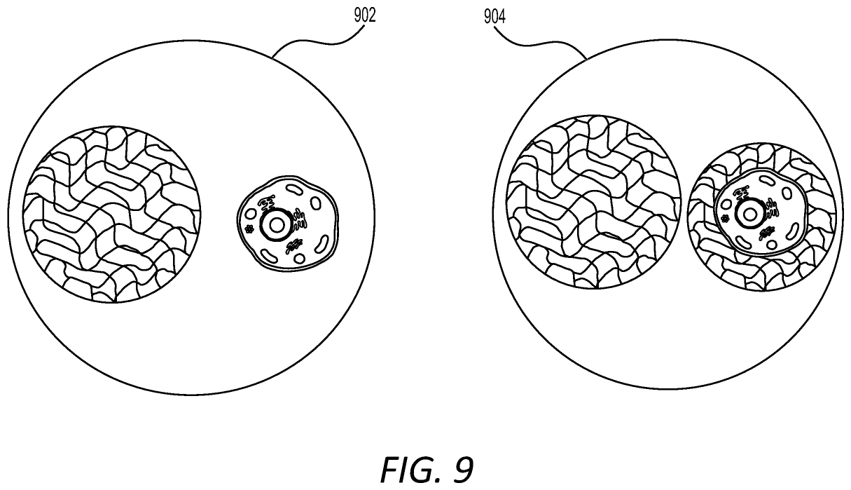

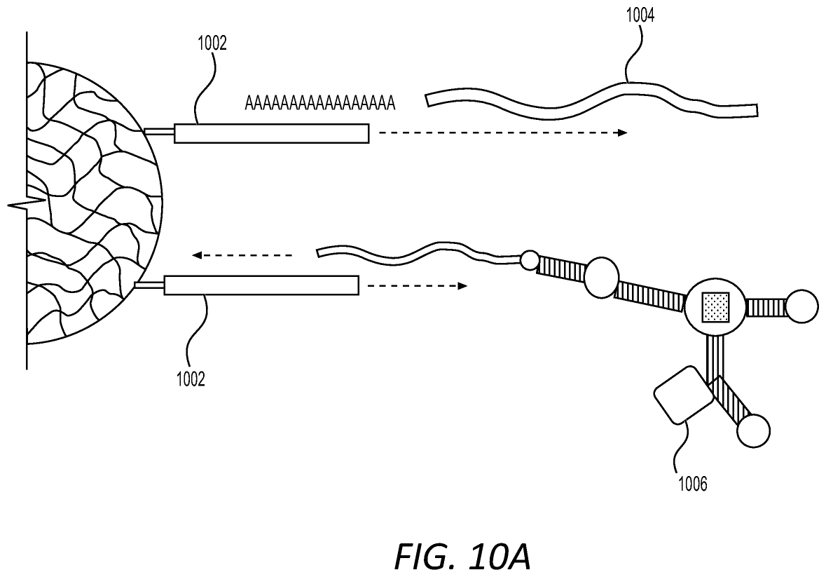

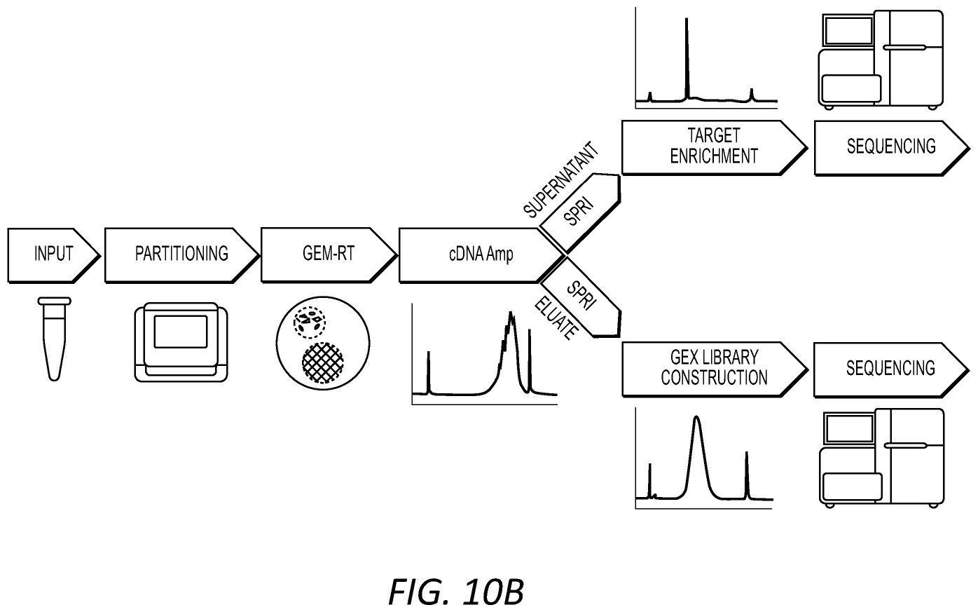

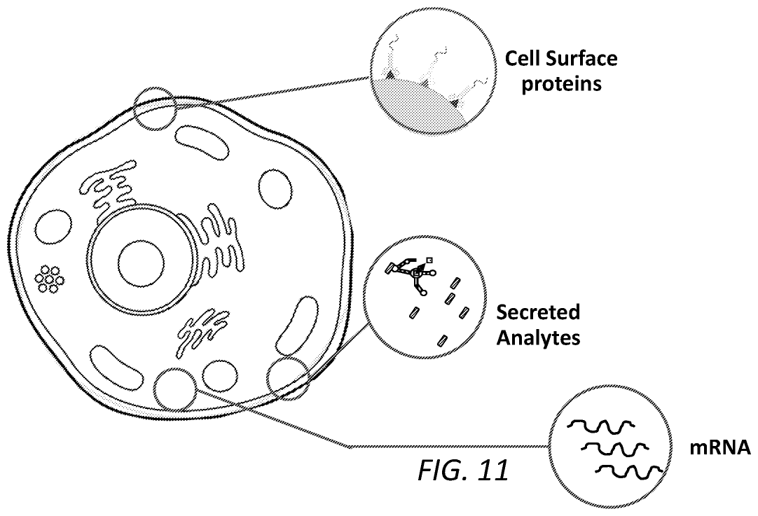

In some embodiments, to facilitate sample multiplexing, individual samples can be stained with lipid tags, such as cholesterol-modified oligonucleotides (CMOs, see, e.g., FIG. 7), anti-calcium channel antibodies, or anti-ACTB antibodies. Non-limiting examples of anti-calcium channel antibodies include anti-KCNN4 antibodies, anti-BK channel beta 3 antibodies, anti-a1B calcium channel antibodies, and anti-CACNA1A antibodies. Examples of anti-ACTB antibodies suitable for the methods of the disclosure include, but are not limited to, mAbGEa, ACTN05, AC-15, 15G5A11/E2, BA3R, and HHF35.

As described elsewhere herein, libraries of labelling agents can be associated with a particular cell/nucleus feature as well as be used to identify analytes as originating from a particular cell/nucleus population, or sample. Cell or nucleus populations can be incubated with a plurality of libraries such that a cell or cells (or nucleus or nuclei) include multiple labelling agents. For example, a cell or nucleus can include coupled thereto a lipophilic labeling agent and an antibody. The lipophilic labeling agent can indicate that the cell or nucleus is a member of a particular cell/nucleus sample, whereas the antibody can indicate that the cell or nucleus includes a particular analyte. In this manner, the reporter oligonucleotides and labelling agents can allow multi-analyte, multiplexed analyses to be performed.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents can be achieved through any of a variety of direct or indirect, covalent or non-covalent associations or attachments. For example, reporter oligonucleotides can be covalently attached to a portion of a labelling agent (such as a protein or nucleic acid molecule, e.g., an antibody, antibody fragment or aptamer) using chemical conjugation techniques (e.g., Lightning-Link® antibody labelling kits available from Innova Biosciences), as well as other non-covalent attachment mechanisms, e.g., using biotinylated antibodies (or biotinylated antigens, or biotinylated antigen fragments) and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent (e.g., an antibody, an antibody fragment, an aptamer, an antigen) is presented as a monomer. In some cases, the labelling agent is presented as a multimer. In some cases, a labelling agent (e.g., an antibody, an antibody fragment, an aptamer, an antigen) is presented as a dimer. In some cases, a labelling agent (e.g., an antibody, an antibody fragment, an aptamer, an antigen) is presented as a trimer. In some cases, a labelling agent (e.g., an antibody, an antibody fragment, an aptamer, an antigen) is presented as a tetramer. In some cases, a labelling agent (e.g., an antibody, an antibody fragment, an aptamer, an antigen) is presented as a pentamer. In some cases, a labelling agent (e.g., an antibody, an antibody fragment, an aptamer, an antigen) is presented as a hexamer. In some cases, a labelling agent (e.g., an antibody, an antibody fragment, an aptamer, an antigen) is presented as a heptamer. In some cases, a labelling agent (e.g., an antibody, an antibody fragment, an aptamer, an antigen) is presented as an octamer. In some cases, a labelling agent (e.g., an antibody, an antibody fragment, an aptamer, an antigen) is presented as a nonamer. In some cases, a labelling agent (e.g., an antibody, an antibody fragment, an aptamer, an antigen) is presented as a decamer. In some cases, a labelling agent (e.g., an antibody, an antibody fragment, an aptamer, an antigen) is presented as a 10+-mer.

In some cases, the labelling agent can include a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

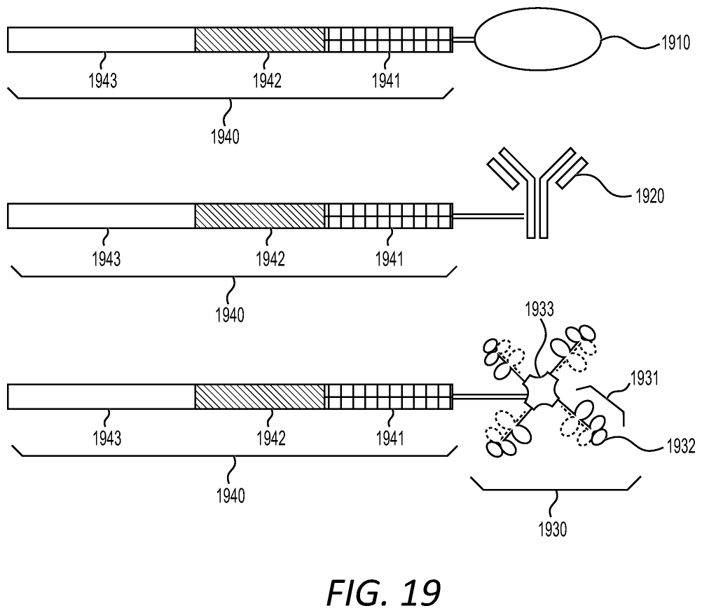

FIG. 19 describes exemplary labelling agents (1910, 1920, 1930) including reporter oligonucleotides (1940) attached thereto. Labelling agent 1910 (e.g., any of the labelling agents described herein) is attached (either directly, e.g., covalently attached, or indirectly) to reporter oligonucleotide 1940. Reporter oligonucleotide 1940 can include barcode sequence 1942 that identifies labelling agent 1910. Reporter oligonucleotide 1940 can also include one or more functional sequences 1943 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer binding sequence (such as an R1, R2, or partial R1 or R2 sequence).

Referring to FIG. 19, in some instances, reporter oligonucleotide 1940 conjugated to a labelling agent (e.g., 1910, 1920, 1930) includes a functional sequence 1941, a reporter barcode sequence 1942 that identifies the labelling agent (e.g., 1910, 1920, 1930), and reporter capture handle 1943. Reporter capture handle sequence 1943 can be configured to hybridize to a complementary sequence, such as a complementary sequence present on a nucleic acid barcode molecule 1990 (not shown), such as those described elsewhere herein. In some instances, nucleic acid barcode molecule 1990 is attached to a support (e.g., a bead, such as a gel bead), such as those described elsewhere herein. For example, nucleic acid barcode molecule 1990 can be attached to the support via a releasable linkage (e.g., including a labile bond), such as those described elsewhere herein. In some instances, reporter oligonucleotide 1940 includes one or more additional functional sequences, such as those described above.

In some instances, the labelling agent 1910 is a protein or polypeptide (e.g., an antigen or prospective antigen, or a fragment of an antigen or prospective antigen) including reporter oligonucleotide 1940. Reporter oligonucleotide 1940 includes reporter barcode sequence 1942 that identifies polypeptide 1910 and can be used to infer the presence of an analyte, e.g., a binding partner of polypeptide 1910 (i.e., a molecule or compound to which polypeptide 1910 can bind, e.g., an antibody or an antigen-binding fragment thereof). In some instances, the labelling agent 1910 is a lipophilic moiety (e.g., cholesterol) including reporter oligonucleotide 1940, where the lipophilic moiety is selected such that labelling agent 1910 integrates into a membrane of a cell or nucleus. Reporter oligonucleotide 1940 includes reporter barcode sequence 1942 that identifies lipophilic moiety 1910 which in some instances is used to tag nuclei or cells (e.g., groups of cells, cell samples, etc.) and can be used for multiplex analyses as described elsewhere herein. In some instances, the labelling agent is an antibody 1920 (or an epitope binding fragment thereof) including reporter oligonucleotide 1940. Reporter oligonucleotide 1940 includes reporter barcode sequence 1942 that identifies antibody 1920 and can be used to infer the presence of, e.g., a target of antibody 1920 (i.e., a molecule or compound to which antibody 1920 binds). In other embodiments, labelling agent 1930 includes an MHC molecule 1931 including peptide 1932 and reporter oligonucleotide 1940 that identifies peptide 1932. In some instances, the MHC molecule is coupled to a support 1933. In some instances, support 1933 can be a polypeptide, such as streptavidin, or a polysaccharide, such as dextran. In some instances, reporter oligonucleotide 1940 can be directly or indirectly coupled to MEW labelling agent 1930 in any suitable manner. For example, reporter oligonucleotide 1940 can be coupled to MEW molecule 1931, support 1933, or peptide 1932. In some embodiments, labelling agent 1930 includes a plurality of MHC molecules, (e.g. is an MHC multimer, which can be coupled to a support (e.g., 1933)). There are many possible configurations of Class I and/or Class II MHC multimers that can be utilized with the compositions, methods, and systems disclosed herein, e.g., MEW tetramers, MHC pentamers (MHC assembled via a coiled-coil domain, e.g., Pro5® MHC Class I Pentamers, (ProImmune, Ltd.), MHC octamers, MEW dodecamers, MHC decorated dextran molecules (e.g., MEW Dextramer® (Immudex)), etc. For a description of exemplary labelling agents, including antibody and WIC-based labelling agents, reporter oligonucleotides, and methods of use, see, e.g., U.S. Pat. No. 10,550,429 and U.S. Pat. Pub. 20190367969.

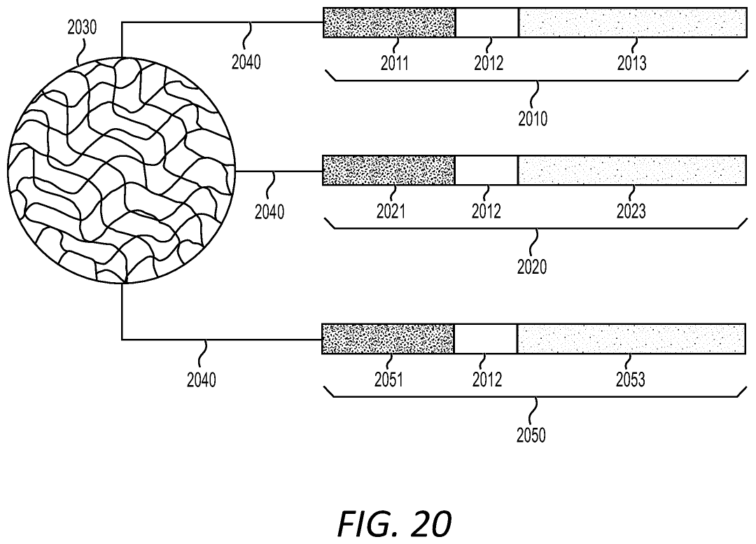

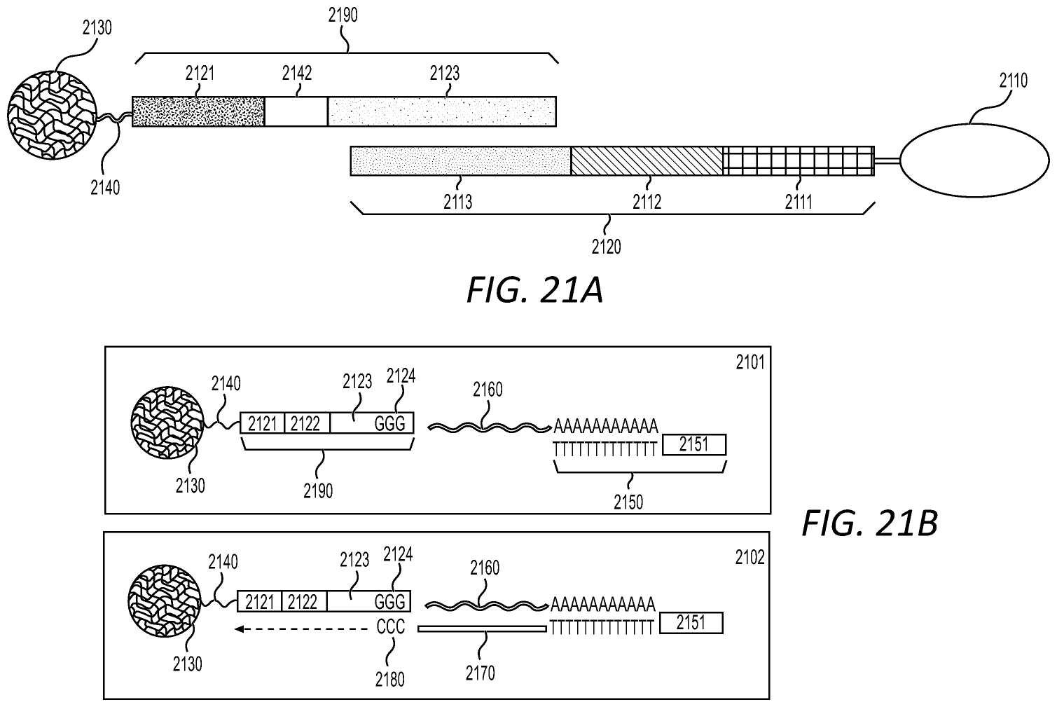

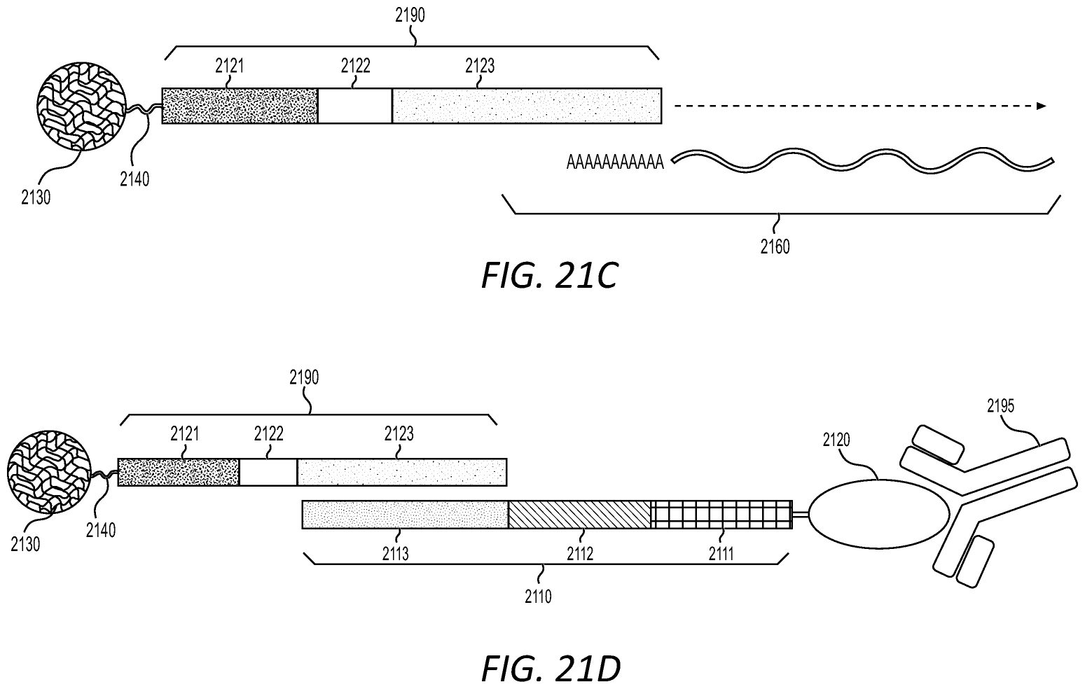

Exemplary barcode molecules attached to a support (e.g., a bead) is shown in FIG. 20. In some embodiments, analysis of multiple analytes (e.g., RNA and one or more analytes using labelling agents described herein) can include nucleic acid barcode molecules as generally depicted in FIG. 20. In some embodiments, nucleic acid barcode molecules 2010 and 2020 are attached to support 2030 via a releasable linkage 2040 (e.g., including a labile bond) as described elsewhere herein. Nucleic acid barcode molecule 2010 can include functional sequence 2011, barcode sequence 2012 and capture sequence 2013. Nucleic acid barcode molecule 2020 can include adapter sequence 2021, barcode sequence 2012, and capture sequence 2023, wherein capture sequence 2023 includes a different sequence than capture sequence 2013. In some instances, adapter 2011 and adapter 2021 include the same sequence. In some instances, adapter 2011 and adapter 2021 include different sequences. Although support 2030 is shown including nucleic acid barcode molecules 2010 and 2020, any suitable number of barcode molecules including common barcode sequence 2012 are contemplated herein. For example, in some embodiments, support 2030 further includes nucleic acid barcode molecule 2050. Nucleic acid barcode molecule 2050 can include adapter sequence 2051, barcode sequence 2012 and capture sequence 2053, wherein capture sequence 2053 includes a different sequence than capture sequence 2013 and 2023. In some instances, nucleic acid barcode molecules (e.g., 2010, 2020, 2050) include one or more additional functional sequences, such as a UMI or other sequences described herein. The nucleic acid barcode molecules 2010, 2020 or 2050 can interact with analytes as described elsewhere herein, for example, as depicted in FIGS. 21A-21C.

Referring to FIG. 21A, in an instance where nuclei or cells are labelled with labeling agents, capture sequence 2123 can be complementary to an adapter sequence of a reporter oligonucleotide. Cells or nuclei can be contacted with one or more reporter oligonucleotide 2120 conjugated labelling agents 2110 (e.g., polypeptide such as an antigen or fragment of an antigen, antibody, or others described elsewhere herein). In some cases, the cells or nuclei can be further processed prior to barcoding. For example, such processing steps can include one or more washing and/or cell sorting steps. In some instances, a cell that is bound to labelling agent 2110 which is conjugated to oligonucleotide 2120 and support 2130 (e.g., a bead, such as a gel bead) including nucleic acid barcode molecule 2190 is partitioned into a partition amongst a plurality of partitions (e.g., a droplet of a droplet emulsion or a well of a microwell array). In some instances, the partition includes at most a single cell bound to labelling agent 2110. In some instances, reporter oligonucleotide 2120 conjugated to labelling agent 2110 (e.g., polypeptide such as an antigen or fragment of an antigen, an antibody, pMHC molecule such as an MHC multimer, etc.) includes a first functional sequence 2111 (e.g., a primer sequence), a barcode sequence 2112 that identifies the labelling agent 2110 (e.g., the polypeptide such as an antigen or fragment of an antigen, antibody, or peptide of a pMHC molecule or complex), and a capture handle sequence 2113. Capture handle sequence 2113 can be configured to hybridize to a complementary sequence, such as capture sequence 2123 present on a nucleic acid barcode molecule 2190 (e.g., partition-specific barcode molecule). In some instances, oligonucleotide 2110 includes one or more additional functional sequences, such as those described elsewhere herein.

Barcoded nucleic acid molecules can be generated (e.g., via a nucleic acid reaction, such as nucleic acid extension, reverse transcription, or ligation) from the constructs described in FIGS. 21A-21C. For example, capture handle sequence 2113 can then be hybridized to complementary capture sequence 2123 to generate (e.g., via a nucleic acid reaction, such as nucleic acid extension or ligation) a barcoded nucleic acid molecule including cell (or nucleus) barcode (e.g., common barcode or partition-specific barcode) sequence 2122 (or a reverse complement thereof) and reporter barcode sequence 2112 (or a reverse complement thereof). In some embodiments, the nucleic acid barcode molecule 2190 (e.g., partition-specific barcode molecule) further includes a UMI. Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) can be performed. For example, the workflow can include a workflow as generally depicted in any of FIGS. 21A-21C, or a combination of workflows for an individual analyte, as described elsewhere herein. For example, by using a combination of the workflows as generally depicted in FIGS. 21A-21C, multiple analytes can be analyzed.

In some instances, analysis of an analyte (e.g. a nucleic acid, a polypeptide, a carbohydrate, a lipid, etc.) includes a workflow as generally depicted in FIGS. 21A and/or 21B. A nucleic acid barcode molecule 2190 can be co-partitioned with the one or more analytes. In some instances, nucleic acid barcode molecule 2190 is attached to a support 2130 (e.g., a bead, such as a gel bead or a solid bead, e.g., a magnetic bead), such as those described elsewhere herein. For example, nucleic acid barcode molecule 2190 can be attached to support 2130 via a releasable linkage 2140 (e.g., including a labile bond) or linker (such as a non-releasable linker), such as those described elsewhere herein. In one embodiment, the support 2130 may be a solid bead (e.g., a magnetic bead) with nucleic acid barcode molecule 2190 attached via a linker (such as a non-releasable linker). Nucleic acid barcode molecule 2190 can include a functional sequence 2121 and optionally include other additional sequences, for example, a barcode sequence 2122 (e.g., common barcode, partition-specific barcode, or other functional sequences described elsewhere herein), and/or a UMI sequence 2125. The nucleic acid barcode molecule 2190 can include a capture sequence 2123 that can be complementary to another nucleic acid sequence, such that it can hybridize to a particular sequence.

For example, capture sequence 2123 can include a poly-T sequence and can be used to hybridize to mRNA. Referring to FIG. 21C, in some embodiments, nucleic acid barcode molecule 2190 includes capture sequence 2123 complementary to a sequence of RNA molecule 2160 from a cell. In some instances, capture sequence 2123 includes a sequence specific for an RNA molecule. Capture sequence 2123 can include a known or targeted sequence or a random sequence. In some instances, a nucleic acid extension reaction can be performed, thereby generating a barcoded nucleic acid product including capture sequence 2123, the functional sequence 2121, UMI sequence 2125, any other functional sequence, and a sequence corresponding to the RNA molecule 2160.

In another example, capture sequence 2123 can be complementary to an overhang sequence or an adapter sequence that has been appended to an analyte. For example, referring to FIG. 21B, in some embodiments, primer 2150 includes a sequence complementary to a sequence of nucleic acid molecule 2160 (such as an RNA encoding for a BCR sequence) from a biological particle. In some instances, primer 2150 includes one or more sequences 2151 that are not complementary to RNA molecule 2160. Sequence 2151 can be a functional sequence as described elsewhere herein, for example, an adapter sequence, a sequencing primer sequence, or a sequence the facilitates coupling to a flow cell of a sequencer. In some instances, primer 2150 includes a poly-T sequence. In some instances, primer 2150 includes a sequence complementary to a target sequence in an RNA molecule. In some instances, primer 2150 includes a sequence complementary to a region of an immune molecule, such as the constant region of a T cell receptor (TCR) or B cell receptor (BCR) sequence. Primer 2150 is hybridized to nucleic acid molecule 2160 and complementary molecule 2170 is generated. For example, complementary molecule 2170 can be cDNA generated in a reverse transcription reaction. In some instances, an additional sequence can be appended to complementary molecule 2170. For example, the reverse transcriptase enzyme can be selected such that several non-templated bases 2180 (e.g., a poly-C sequence) are appended to the cDNA. In another example, a terminal transferase can also be used to append the additional sequence. Nucleic acid barcode molecule 2190 includes a sequence 2124 complementary to the non-templated bases, and the reverse transcriptase performs a template switching reaction onto nucleic acid barcode molecule 2190 to generate a barcoded nucleic acid molecule including cell or nucleus (e.g., partition specific) barcode sequence 2122 (or a reverse complement thereof) and a sequence of complementary molecule 2170 (or a portion thereof). In some instances, capture sequence 2123 includes a sequence complementary to a region of an analyte that is highly conserved. Capture sequence 2123 is hybridized to nucleic acid molecule 2160 and a complementary molecule 2170 is generated. For example, complementary molecule 2170 can be generated in a reverse transcription reaction generating a barcoded nucleic acid molecule including cell or nucleus barcode (e.g., common barcode or partition-specific barcode) sequence 2122 (or a reverse complement thereof) and a sequence of complementary molecule 2170 (or a portion thereof). Additional methods and compositions suitable for barcoding cDNA generated from mRNA transcripts and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969.

In some instances, the analysis of a secreted antibody comprises a workflow as depicted in FIG. 21D. Cells may be contacted with one or more second reporter agents (as further described herein) that comprise reporter oligonucleotides 2110. A nucleic acid barcode molecule 2190 may be co-partitioned with a cell (e.g., a plasma B cell). In some instances, a secreted antibody 2195 (coupled to the surface of the cell via a first reporter agent which is not shown in FIG. 21D) that is bound to the second reporter agent's target analyte 2120 which is conjugated to oligonucleotide 2110 and support 2130 (e.g., a bead, such as a gel bead or a solid bead) comprising nucleic acid barcode molecule 2190 is partitioned into a partition amongst a plurality of partitions (e.g., a droplet of a droplet emulsion or a well of a microwell array). In some instances, the partition includes at most a single cell with the second reporter agent coupled to an antibody and the antibody coupled to the surface of the cell via a first reporter agent. In some instances, reporter oligonucleotide 2110 conjugated to the second reporter agent's target analyte 2120 (e.g., a protein or polypeptide, such as an antigen or prospective antigen) comprises a first adapter sequence 2111 (e.g., a primer sequence), a barcode sequence 2112 that identifies the target analyte of the second reporter agent 2120 (e.g., a protein or polypeptide, such as an antigen or prospective antigen), and an adapter sequence 2113. Adapter sequence 2113 may be configured to hybridize to a complementary sequence, such as sequence 2123 present on a nucleic acid barcode molecule 2190. The workflow depicted in FIG. 21D for secreted antibodies may be performed simultaneous with the workflow depicted in FIG. 21B, e.g., for mRNA processing, as further described herein. In one embodiment, the combined workflow provides secreted antibody and mRNA transcript (including those encoding V(D)J regions of immune cell receptors) information on a single cell level. In this case, sequences 2123 of the nucleic acid barcode molecule 2190 are configured to couple to both the complementary molecule 2170 generated as depicted in FIG. 21B (e.g., via sequence 2124) and the adapter sequence 2113.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620(2020).

Gene expression data can reflect the underlying genome and mutations and structural variants therein. As a result, the variation inherent in the captured and sequenced RNA molecules can be used to identify genotypes de novo or used to assign molecules to genotypes that were known a priori. In some embodiments, allelic variation that is present due to haplotypic states (including linkage disequilibrium of the human leucocyte antigen loci (HLA), immune receptor loci (BCR), and other highly polymorphic regions of the genome), can also be used for demultiplexing. Expressed B cell receptors can be used to infer germline alleles from unrelated individuals, which information may be used for demultiplexing.

### Combinatorial Barcoding

In some instances, barcoding of a nucleic acid molecule may be done using a combinatorial approach. In such instances, one or more nucleic acid molecules (which may be comprised in a cell, e.g., a fixed cell, or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating first barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The first barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the first barcoded nucleic acid molecules, thereby generating second barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The second barcoded nucleic acid molecules may thus comprise two barcode sequences (e.g., from the first nucleic acid barcode molecules and the second nucleic acid barcode molecules).

Additional barcode sequences may be attached to the second barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 10⁶ unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a first barcoded nucleic acid molecule. A second unique nucleic acid molecule comprising one or more nucleic acid bases may be attached to the first barcoded nucleic acid molecule molecule, thereby generating a second barcoded nucleic acid molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety.

### Kits

Further provided herein are kits, which may be useful for the practice of a method, or generating the partition provided in the methods, described herein. In some embodiments, the kits are for (i) detecting secretion of an analyte by a cell (or a nucleus), (ii) characterizing an analyte secretion profile of a cell (or a nucleus) (iii) identifying a cell as a cell of interest (or a nucleus as a nucleus of interest); or (iv) enriching for a cell as a cell of interest (or a nucleus as a nucleus of interest). In one embodiment, the kit may include: (a) instructions for use and (b) a reporter agent. The reporter agent may include a first aptamer including first and second domains in which the first domain may bind the secreted analyte and the second domain may be responsive to the analyte binding to the first domain. In some embodiments, the second domain may include a subdomain capable of uptake of a detectable moiety upon the analyte binding to the first domain. In other embodiments, the second domain may include a subdomain capable of release of a detectable moiety in response to the analyte binding to the first domain.

The first aptamer of the reporter agent may further include a third domain response to the analyte binding to the first domain that includes a reporter oligonucleotide. The reporter oligonucleotide may include a reporter barcode sequence specific for the first aptamer (that identifies the first aptamer, e.g. aptamer capable of binding the analyte or the aptamer's analyte) and a capture handle sequence.

The secreted analyte, to which the first domain of the first aptamer binds may be, for example, a peptide, a nucleic acid, or a lipid molecule analyte. If the secreted analyte is a peptide, it may be a cytokine or a growth factor. For instance, if the secreted analyte is a cytokine, it may be tumor necrosis factor (TNF)α, cluster of differentiation (CD)27, CD30, CD40, interferon (IFN)-α, IFN-β, IFN-γ, IFN-α, IFN-γ, interleukin (IL)-1, IL-2, IL-4, IL-6, IL-10, IL-13, IL-17RA, IL-17RB, IL-17RC, IL17RD, IL-17RE, IL-22, macrophage (M)-colony stimulating factor (CSF), granulocyte (G)M-CSF, or a chemokine. If the secreted analyte is a growth factor, the secreted analyte may be insulin growth factor (IGF), epidermal growth factor (EGF), platelet-derived growth factor (PDGF) or fibroblast growth factor (FGF). If the secreted analyte is a peptide it may also be a hormone or any other analyte secreted by a cell associated with a disease, such as inflammatory disease, cancer, or infectious disease. The secreted analyte may also be any analyte that may expected to be associated with a cell of a particular type that would be useful for its selection and/or enrichment relative to or from other cell types. In some embodiments, if the analyte is a peptide, it is not an antibody, antibody fragment or secreted fragment of a B or T cell receptor. Furthermore, if the secreted analyte is a nucleic acid, it may be an RNA, and may be a microRNA. If the secreted analyte is a lipid, it may be a lipid associated with a vesicle, microvesicle or exosome.

The first aptamer of the reporter agent in the kit may further include a lipophilic moiety, e.g., for embedding the reporter agent in the cell or nuclear membrane, and/or may also include a cell or nucleus binding domain capable of binding to a cell or nuclear membrane protein. If the first aptamer further includes the cell or nucleus binding domain, the binding domain may be capable of binding to a cell membrane protein, such as a cytoskeleton protein or a cluster of differentiation protein, e.g., CD45, or beta actin, or a nuclear membrane protein.

In the kit, the first aptamer of the reporter agent may be linked (e.g., ligated) to a second aptamer, or the kit may further include, as a separate reagent, a second aptamer. If the second aptamer is further included in the kit, the first aptamer may include a first linking sequence (e.g., first ligation handle sequence) and the second aptamer may include a second linking sequence (e.g., second ligation handle sequence) wherein the first and the second linking sequences are capable of being linked (e.g., ligated by complementary base pairing), to thereby link the first aptamer to the second aptamer. The second aptamer may further include a second reporter oligonucleotide and a lipophilic moiety that embeds the second aptamer in the cell or nuclear membrane. The second reporter oligonucleotide may include a second reporter barcode specific for the second aptamer (e.g., specific for the second aptamer, or aptamer capable of embedding in the cell or nuclear membrane) sequence and a capture handle sequence. The second aptamer may also be capable of binding a cell surface protein, such as a cytoskeleton protein, a CD protein or beta actin, or a nuclear membrane protein.

Any of the kits described herein may also include a plurality of nucleic acid barcode molecules. The plurality of nucleic acid barcode molecules may include a capture sequence, which may be complementary to a capture handle sequence to any of the first and/or second reporter oligonucleotides. In some aspects, one of either the reporter oligonucleotides or the plurality of nucleic acid barcode molecules may further include a unique molecular identifier (UMI).

Any of the kits provided herein may also include a detectable moiety, detectable moiety which may be bound to or associated with or coupled to the first aptamer at its second domain's subdomain. If a detectable moiety is further included in the kit it may be a fluorophore, chromophore, heavy metal, radionuclide or any combinations thereof. Examples of fluorophore that can be utilized here are 3,5-difluoro-4-hydroxybenzylidene imidazolinone (DFHBI), thiazole orange, 3,5-difluoro-4-hydroxynenzylidene imidazolino-2-oxime (DFHO), cyanine (Cy3, Cy5), fluorescein isothiocyanate (FITC), phycoerythrin (PE), allophycocyanin (APC), ALexa Four, DyLight, and etc.

Further, the kits may further include one or more agents to stimulate or inhibit secretion of the analyte. The agents include any one or more of feeder cells, a small molecule drug, a cytokine (that is not the secreted analyte), a Pattern recognition receptor (PRR) ligand (e.g., Toll-like receptor (TLR) ligand, NOD-like receptor (NLR) ligand, RIG-I-like receptor (RLR) ligands C-type lectin receptor (CLR) ligand, cytosolic dsDNA sensor (CDS) ligand, etc.), lipopolysaccharide (LPS), double-stranded DNA (dsDNA), double-stranded RNA (dsRNA), a synthetic dsRNA (e.g., polyinosinic-polycytidylic acid (poly I:C) or polyadenylic-polyuridylic acid (poly(A:U)), CpG oligodeoxynucleotides (CpG ODN), or any combination thereof. The agent may be coupled to a major histocompatibility complex (MHC) molecule. In some cases, the MHC molecule may be an MHC multimer (e.g., a monomer, dimer, trimer, tetramer, pentamer, etc.) resulting in a multimeric (e.g., a monomeric, dimeric, trimeric, tetrameric, etc.) WIC-antigen complex (e.g., MHC-peptide complex if the antigen is a peptide). The WIC multimer may be linked to a cell or a polymer. The cell may be an antigen-presenting cell (APC). The polymer may be a dextran polymer (e.g., a dextramer). An WIC multimer (e.g., tetramer or dextramer) or an APC may comprise a plurality of WIC complexes. The MHC molecules/multimers may comprise one or more stimulatory molecules, such as antigenic peptides, thereby forming peptide-MHC complexes. Thus, WIC molecules (e.g., multimers) and/or APCs may be used to present stimulatory and/or co-stimulatory molecules (e.g., stimulatory peptides via MHC-peptide complexes) to the cell (e.g., an immune cell) to induce secretion of the one or more analytes. Moreover, cell or other non-cell constructs may be used to present stimulatory molecules to a cell to induce secretion of one or more analytes. In some cases, an antigen-presenting cell (APC) may comprise a plurality of WIC molecules (e.g., MHC multimers), and thus an APC may be used to induce analyte secretion of a cell (e.g., an immune cell). A co-stimulator molecule as described herein may be an antibody (e.g., an anti-CD3 or an anti-CD28 antibody) or a cytokine (e.g., an interleukin). An APC or an WIC multimer (such as a tetramer or dextramer), for example, may comprise a plurality of co-stimulatory molecules and thus may be used to induce analyte secretion from a cell. In some cases, the stimulator or co-stimulatory molecules, the WIC multimer, the APC, and/or the MHC molecules as described herein may comprise a nucleic acid molecule comprising a barcode sequence. In some cases, methods disclosed herein comprise antigens being part of an antigen-MHC complex (e.g., an antigen-MHC tetramer) comprising the antigen (e.g., a peptide or polypeptide) and an MHC molecule (e.g., an WIC multimer such as a tetramer). The WIC molecule may comprise a nucleic acid molecule comprising a barcode sequence that identifies the peptide(s) present in an WIC molecule or multimer. See, e.g., U.S. Pat. No. 10,011,872.

In certain particular embodiments, the kits described herein above, the kit may further include a second reporter agent. The second reporter agent may be included in the kit in instances wherein the analyte is an antibody or antigen-binding fragment thereof and the kit is for detecting secretion of the antibody of antigen-binding fragment thereof by an antibody-secreting cell or is for characterizing the antibody of antigen-binding fragment thereof secreted by the antibody-secreting cell. The second reporter agent, included in such a kit, may include a target antigen, e.g., target antigen to which the antibody or antigen-binding fragment thereof may bind, and a reporter oligonucleotide. The reporter oligonucleotide may include a reporter barcode sequence that is specific to the target antigen and may include a capture handle sequence. The capture handle sequence may couple to a barcode nucleic acid molecule of the plurality of barcode nucleic acid molecules that may be included in the kit as discussed herein, e.g., paragraph 289. Further disclosure related to the second reporter agent, target antigens, and antibodies and antigen-binding fragments thereof can be found in “METHODS OF THE DISCLOSURE” under “Methods for identifying or characterizing a cell as secreting an analyte” earlier herein.

Also included in the kits may be further, additional, reporter agents. Optionally, the kits may include at least a second, at least a third, at least a fourth, at least a fifth, at least a sixth, at least a seventh, at least an eighth, at least a ninth, at least a tenth, at least a fifteenth, at least a twentieth, at least a twenty fifth, at least a thirtieth, at least a fiftieth, at least a one hundredth additional reporter agent may be used to detect at least a second, at least a third, at least a fourth, at least a fifth, at least a sixth, at least a seventh, at least an eighth, at least a ninth, at least a tenth, at least a fifteenth, at least a twentieth, at least a twenty fifth, at least a thirtieth, at least a fiftieth, at least a one hundredth additional secreted analyte, respectively. Furthermore, additional reporter agents not including one or more aptamers may be used to detect cell analytes that may, but are not necessarily, secreted by the cell. Such analytes may be cell surface associated, may be nucleic acids comprised in the cell, e.g., mRNA, or lipids and may be detected by reagents described in the “Further Disclosure—Partitions, Partitioning, Reagents and Processing” section herein. The analytes from the cell may instead be analytes from a nucleus isolated from a cell.

Any of the kits provided herein may further include positive and/or negative control reagents. It will be understood that the control reagents or other reagents as may be needed to process samples.

In some embodiments, the kits may further include enzymes, aqueous or frozen solutions, primers or other reagents, e.g., labeling reagents, as may be desirable for using the kit for its intended purpose. Some of the reagents are described in the “Further Disclosure—Partitions, Partitioning, Reagents and Processing” section, above. The various components of the kit can each be in separate containers, combined in single container, or combined in various container as appropriate.

The kit may also further include instructions for use thereof. The instructions for use may be included a package insert including information concerning the components of the kit by the user and/or informational aids. Generally, informational aids may include proper storage conditions, references, manufacturer/distributor information, compatible systems for use and intellectual property information.

The instructions for may be provided in any format, e.g., they may be recorded on a suitable recording medium. For example, the instructions can be printed on a substrate, such as paper or plastic, etc. The instructions may, alternatively, be present as an electronic storage data file present on a suitable computer readable storage medium, e.g. CD-ROM, diskette, flash drive, etc. In some instances, the instructions are not physically present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

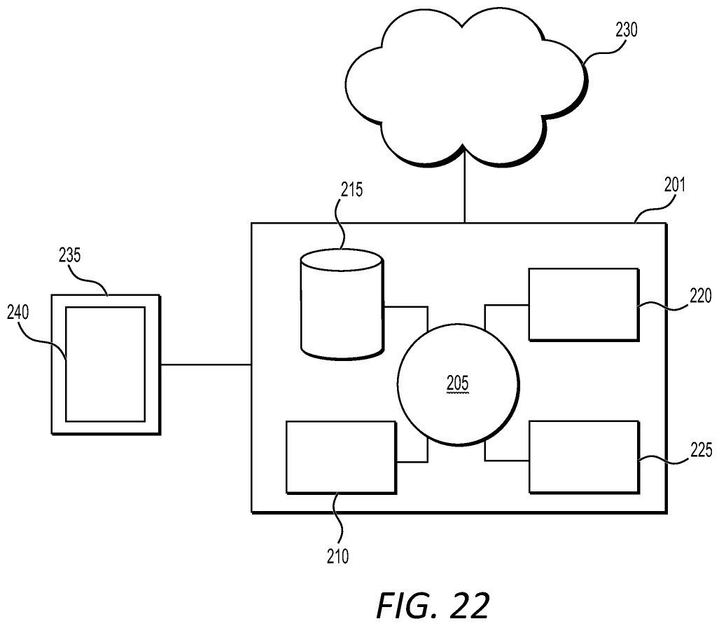

FIG. 22 depicts a block diagram illustrating an example of a computer system 201, in accordance with some example embodiments. Referring to FIG. 22, the computer system 201 may be configured to implement one or more of the analysis engine 2202, the sequencing platform 2204, and the client device 2206. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” generally in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, results of the assay, such as a summary of one or more analytes as being and/or not secreted, by a cell or cells (or nucleus or nuclei). Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, contact a reporter agent with secreted analytes, or identify the secreted analytes that bind the reporter agent, or determine level of secreted analytes, or characterize a cell or nucleus based on an identified and/or level of secreted analytes as described herein.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell or nucleus. For example, a biological particle (e.g., a cell, nucleus or cell bead) is partitioned in a partition (e.g., droplet), and multiple analytes from the biological particle are processed for subsequent processing. The multiple analytes may be from the single cell or nucleus. This may enable, for example, simultaneous proteomic, transcriptomic and genomic analysis of the cell or nucleus.

All publications and patent applications mentioned in this disclosure are herein incorporated by reference to the same extent as if each individual publication or patent application was specifically and individually indicated to be incorporated by reference. To the extent publications and patents or patent applications incorporated by reference contradict the disclosure contained in the specification, the specification is intended to supersede and/or take precedence over any such contradictory material.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1

### Barcoding Analytes from a Single Cell

This example illustrates the production and identification of barcoded, secreted analytes from cells in vitro, using a reporter agent.

Partitioned cells (e.g., immune or tumor cells) isolated from a subject are incubated with a solution of reporter agents (e.g., first aptamers) under a sufficient condition so that a plurality of reporter agents (first aptamers) bind to analytes being secreted by a cell. The first aptamer is analyte-specific, and it may be embedded in the cell membrane via its being coupled to a lipophilic moiety and/or its linkage to, conjugation to, or linkage (e.g., ligation) to a second aptamer coupled to a lipophilic moiety and/or that targets a cell surface protein. The analyte-specific first aptamer may be conjugated to a known DNA oligonucleotide sequence (e.g., reporter barcode sequence). Prior to, or following, partitioning of the cells, the cells are stimulated with specific agents or other suitable stimuli under a sufficient condition to facilitate or allow secretion of analytes (e.g., cytokines or other intracellular analytes), as shown herein.

Further, the reporter agents specific to the secreted analytes may include a domain responsive to the analyte binding to the first aptamer. The domain responsive to the analyte binding to the first aptamer may uptake or release a detectable moiety upon the first aptamer binding to the secreted analyte. The detectable moiety may be a fluorophore, chromophore, heavy metal, radionuclide or combinations thereof. Furthermore, the coupling chemistry and type of covalent bond between the reporter agent and these labels may be altered. Optionally, DNA oligonucleotide barcoded antibodies specific to cell surface proteins are also added for further cell analysis at this stage.

In some examples, the partitions may be in a well or a droplet and the partitions may comprise beads that comprise a different barcode (e.g., a partition/cell-specific barcode) that is different from the analyte-specific barcodes conjugated to the reporter agents. Cells may be lysed inside the partitions to release intracellular contents and generate two different types of barcodes (e.g., analyte-specific barcodes and partition-specific/cell-specific barcodes). DNA amplifications and sequencing of the barcodes may be performed for the measurement of the secreted molecules (e.g., cytokines and other analytes). As a result, the secreted molecules (e.g., cytokines and other analytes) may be identified and quantified.

### Example 2

### Barcoding Analytes from a Single Cell Using Cell Beads

This example illustrates the production and identification of barcoded, secreted analytes from cells in vitro, using cell beads.

Cells (e.g., immune cells) are isolated from a subject, and each cell is further encapsulated into a droplet, which subsequently gels into a hydrogel matrix. The polymeric precursors are subjected to conditions sufficient to polymerize the precursors, such that cell beads are generated, each comprising a single cell.

Cell beads are then placed in a solution comprising specific agents or other suitable stimuli under a sufficient condition to facilitate or allow secretion of analytes (e.g., cytokines or other analytes). Further, the cell beads are incubated in another solution comprising a reporter agents with reporter molecules (e.g., first aptamers), which have specific affinity to the secreted analytes (e.g., cytokines or other secreted analytes). In some examples, a co-stimulatory agent such as CD3 is added to cells to induce secretion of an analyte. Reporter agents specific to the secreted analytes may include a domain responsive to the analyte binding to the first aptamer. The domain responsive to the analyte binding to the first aptamer may uptake or release a detectable moiety upon the first aptamer binding to the secreted analyte. The detectable moiety may be a fluorophore, chromophore, heavy metal, radionuclide or combinations thereof. Furthermore, the coupling chemistry and type of covalent bond between the reporter agent and these labels may be altered. Optionally, DNA oligonucleotide barcoded antibodies specific to cell surface proteins are also added for further cell analysis at this stage.

A partition of a cell bead (e.g., into an emulsion droplet) and may differ from other partitions including other beads in that the other partitions may comprise different barcodes (e.g., partition/cell-specific barcodes) and may comprise different analyte-specific barcodes conjugated to the reporter agents. Cells may be lysed inside the partitions to release intracellular contents and generate two different types of barcodes (e.g., analyte-specific barcodes and partition-specific/cell-specific barcodes). DNA amplifications and sequencing of the barcodes may be performed for the measurement of the secreted molecules (e.g., cytokines and other intracellular analytes). As a result, the secreted analytes (e.g., cytokines and other analytes) may be identified and quantified.

### Example 3

### Staining Analytes from a Single Cell Using Cell Beads

This example describes the staining of barcoded-secreted molecules.

Cell beads each comprising a single cell are formed. After incubation with specific agents or other suitable stimuli under a sufficient condition, the cell beads are treated with suitable enzymes under suitable conditions so that the extra-cellular matrix (ECM) comprising of each bead is partially digested. Further, the partially digested cell beads are incubated with a plurality of barcoded reporter agents with reporter molecules (e.g., barcoded first aptamer), which have specific affinity to the secreted analytes (e.g., cytokines or other secreted analytes). The partially digested ECM allows the barcoded reporter agent to bind to secreted analyte (e.g., cytokine or other secreted analytes). In addition, washing away the excess reporter agents may be performed for imaging of the secreted molecules (e.g., cytokines or analytes).

### Example 4

### Identifying and Quantifying Secreted Analytes from a Single Cell by Barcoded Nucleic Acid Molecules

This example describes the analytical processing of barcoded-secreted molecules.

Once the barcoding procedure outlined in Examples 1 and 2 is complete, the emulsion droplets are coalesced into a bulk solution. The amplified cDNAs and reporter agent-derived DNA oligonucleotides are separated by size, and are then independently converted into Illumina sequencing libraries. The partition-specific DNA barcode computationally infers that all mRNA and reporter agent-derived DNA oligonucleotides possessing the same sequence derived from the same single cell (e.g., immune or tumor cell). DNA amplifications and sequencing of the analyte-specific polypeptide DNA barcodes (e.g., reporter barcode sequence) identifies and quantifies the secreted analyte. In some examples, there may be simultaneous measurement of secreted analytes, mRNAs and cell surface proteins.

### Example 5

### Assessing Cytokine Release Potential of a Monoclonal Antibody

This example demonstrates the quantification of cytokine release induced by a given monoclonal antibody, and identification of the cellular populations responsible for this cytokine release, using single cell technologies.

One or more wells of a plate are coated with the monoclonal antibody of interest (e.g., a candidate monoclonal antibody). As a positive control, monoclonal antibodies targeting CD3 and/or CD28, or phorbol 12-myristate and ionomycin, may be added to one or more wells of the same plate. Additional wells within the same plate may be coated with an irrelevant monoclonal antibody, human myoglobin, and/or human hemoglobin to serve as a negative control. A cell of an immune sample of interest (e.g., PBMCs, tumor biopsy, or another immune sample) is then added to each well.

Reporter agents are then added to the plate. The reporter agents are comprised of a first aptamer that includes a domain specific for and that binds to the analyte, a domain comprising a reporter oligonucleotide and a domain that is responsive to the analyte binding to the domain that binds to the analyte. In some examples, the reporter agent could also be lipid-conjugated, enabling insertion of the reporter agent into the cell membrane of the cells. In some embodiments, the first aptamer can be ligated to a second aptamer via a first linking sequence (e.g., first ligation handle sequence) of the first aptamer and a second linking sequence (e.g., second ligation handle sequence) of the second aptamer. The second aptamer may be lipid-conjugated and/or may have a domain that binds to a cell surface protein, enabling insertion of the reporter agent into the cell membrane of the cells.

Cells and stimulatory agents are incubated for 6, 12, and 18 hours in parallel, in replicates. During this time, the reporter agent binds secreted cytokines from the cells.

The detection of released cytokine is measured by detection of the reporter barcode oligonucleotides within cell-associated partitions (e.g., gel beads in emulsions), allowing for or facilitating the detected cytokine to be traced back to a single cell. In some examples, detection of the reporter barcode oligonucleotide on the reporter agent indicates that a cell has secreted a cytokine. In other examples, in which the first aptamer includes the domain that is responsive to the analyte binding to the analyte binding domain, detecting a response in the domain by the analyte binding to the analyte binding domain indicates that a cell has secreted the cytokine and/or identifies the cell as eligible for further analysis, or selects the cell.

In combination with secretion of the analyte, various other multiomic measurements and analyses may be performed. These include, but are not limited to, gene expression and cell surface protein expression. Optionally, any analyses can be assessed by comparison to the positive and negative control samples.

### Example 6

### Labeling B Cells with a Secreted Antibody Bound to a Reporter Oligonucleotide Labeled Antigen Using Capture Agents

This example illustrates the use of capture agents to study plasma cell antibodies.

A cell population of interest (e.g., antigen-specific memory B cells, plasma cells after immunization or vaccination, or memory B cells) is isolated, or enriched for, by flow cytometry, magnetic enrichment, BAC S, or another alternative method. Isolated B cells are partitioned into droplets to produce single cell suspensions.

The isolated B cell single cell suspension is incubated with capture agent (e.g., an aptamer capture agent). An aptamer capture agent may be any aptamer reagent as described herein that (i) embeds in the cell membrane, e.g., via lipophilic moiety, and/or binds to a cell membrane protein on the surface of a B cell, e.g., CD19, CD20, CD45 or CD22, via a cell protein binding domain and (ii) binds to a constant region of a secreted antibody. The aptamer capture agent may be a one-aptamer or a two-aptamer capture reagent. In examples, a one-aptamer capture agent may include a single aptamer having both a lipophilic moiety (and/or cell membrane protein binding domain) that embeds in (and/or binds to a protein) in the cell membrane and a domain that binds the secreted antibody. In other examples, an aptamer reagent that may be a two-aptamer reagent including a first aptamer that has a lipophilic moiety (and/or cell membrane protein binding domain) for embedding (and/or binding to a protein) in the cell membrane and a second aptamer that binds the antibody, e.g., at a constant region, of the antibody. In examples in which the aptamer capture reagent is a two-aptamer capture reagent, the first and second aptamers may also have first and second linking sequences that can link, e.g., by complementary base pairing, the first to the second aptamer. In any of these aptamer capture agents, an aptamer that binds the antibody may also have a subdomain that uptakes or releases a detectable moiety, upon binding the antibody. Further, for any of these aptamer capture reagents, any aptamer included therein may have a reporter oligonucleotide, wherein the reporter oligonucleotide may have a reporter barcode sequence that can be amplified downstream.

The cells incubated with the capture agent, e.g., aptamer capture reagent, are exposed to one or more reporter barcoded antigens (e.g., antigens coupled to a reporter oligonucleotide having a reporter barcode sequence that identifies the antigen). The reporter barcoded antigens may also be conjugated to a fluorophore. In some examples, plasma cells secreting antibodies that bind to the antigen are further enriched by a secondary enrichment against the antigen, e.g. using the fluorophore if the reporter barcoded antigen is coupled to a fluorophore. In embodiments, polymer matrix of a cell bead can be formed by addition of hydrogen peroxide, when the antigen has a horseradish peroxidase or similar domain conjugated, exclusively producing partitions around cells binding reporter-barcoded antigens of interest.

The cells binding the antigens of interest via the secreted antibodies having been coupled to their surface using aptamer capture reagents are captured and partitioned. The captured cells are partitioned with nucleic acid barcode molecules having a partition-specific barcode sequence. These cells are subject to reactions that generate barcoded nucleic acid molecules, e.g., barcoded nucleic acid molecules including sequences: (i) of the nucleic acid barcode molecules (e.g., the partition-specific barcode sequence) and (ii) encoding antibodies secreted by the cell and/or corresponding in whole or part to a sequence of a reporter oligonucleotide. In some examples, generation of barcoded nucleic acid molecules occurs in the partition. In other examples, generation of barcoded nucleic acid molecules occurs in bulk e.g, after (i) coupling of sequences of the nucleic acid barcode molecules to sequences encoding antibodies of the cell and/or of reporter oligonucleotides and (ii) pooling of the coupled sequences (of the nucleic acid barcode molecules to those encoding antibodies of the cell and/or of reporter oligonucleotides).

In some embodiments, the barcoding, and subsequent V(D)J amplification, is performed using the Immune Profiling Solution of the 10× Genomics Chromium™ system. This produces at least a vector of antigen-specific counts for each antigen, for each captured B cell, enabling the identification of the antigen specificity of that cell.

### Example 7

### Detection and Analysis of Secreted Antibodies

Cells with a reasonable number of antigen-binding counts from Example 6 are identified. Within this group, cells which also have additional multiomic counts (e.g., cellular RNA or surface protein) are identified. Transform the antigen-binding count distribution by subtracting the median or mean background for each antigen as detected in empty droplets. Cells that bind both the antigen of interest and the control antigen, or that bind just the control antigen, are excluded from further analysis. Then, transform the antigen count vector using any number of approaches (e.g., centered log ratio, PCA, GLMPCA, or more complex machine learning models or neural networks). Finally, antibodies with antigen fingerprints are chosen for therapeutic evaluation.

A cDNA library prepared from cells secreting antibodies that bind the antigen of interest are used to selectively amplify for sequences encoding secreted antibodies, e.g., using primers that hybridize to a given cell barcode and antibody heavy chain and light chain constant regions. Separate heavy chain and light chain amplification occurs, followed by overlap extension PCR to combine them into a single continuous molecule. Heavy chain and light chains are directly paired, enabling an antibody with ideal antigen specific to be rapidly cloned and converted into a format amenable to therapeutic development and testing.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. A method of identifying or characterizing a cell as secreting an analyte comprising:
(a) providing a partition comprising,
a cell,
a plurality of nucleic acid barcode molecules comprising a partition-specific barcode sequence; and
a first reporter agent comprising:
a first aptamer comprising a first reporter oligonucleotide, a first linking sequence, and a lipophilic moiety that embeds the first aptamer in the cell membrane;
a second aptamer comprising a second reporter oligonucleotide, a second linking sequence, and a domain that binds the analyte;
wherein the first reporter oligonucleotide comprises a first reporter barcode sequence specific to the first aptamer and a first capture handle sequence,
wherein the second reporter oligonucleotide comprises a second reporter barcode sequence specific to the second aptamer and a second capture handle sequence, and
wherein the first and second capture handle sequences are configured to couple to a capture sequence comprised in a first of the plurality of nucleic acid barcode molecules; and
(b) subjecting the partition to conditions sufficient (i) to embed the lipophilic moiety in the cell membrane of the cell, (ii) to link the first aptamer and second aptamer via the first and second linking sequences, and (iii) to bind a secreted analyte from the cell via the domain of the second aptamer.

2. The method of claim 1, wherein the first and second linking sequences comprise complementary nucleic acid sequences.

3. The method of claim 1 or claim 2, wherein the first and second capture handle sequences couple to the capture sequence by complementary base pairing.

4. The method of any of claims 1-3, further comprising: (c) generating a first and a second barcoded nucleic acid molecule,
wherein the first barcoded nucleic acid molecule comprises a sequence of the first reporter oligonucleotide or reverse complement thereof and the partition-specific barcode sequence or reverse complement thereof, and
wherein the second barcoded nucleic acid molecule comprises a sequence of the second reporter oligonucleotide or reverse complement thereof and the partition-specific barcode sequence or reverse complement thereof.

5. The method of claim 4, further comprising determining sequence of the first barcoded nucleic acid molecule.

6. The method of claim 5, further comprising determining embedding of the first aptamer in the cell membrane of the cell based on the determining the sequence of the first barcoded nucleic acid molecule.

7. The method of any of claims 4-5, further comprising determining sequence of the second barcoded nucleic molecule.

8. The method of claim 4, further comprising identifying or characterizing the cell as secreting the analyte based on the determining the sequence of the second barcoded nucleic acid molecule.

9. The method of claim 5, further comprising:
determining sequence of the second barcoded nucleic acid molecule; and
determining a ratio of determined sequence of the first relative to the second barcoded nucleic acid molecule,
wherein the analyte is coupled to the cell membrane of the cell if the ratio is approximately 1:1.

10. The method of any preceding claim, wherein the first aptamer further comprises a cell protein binding domain,
optionally, wherein the cell protein binding domain binds to a cell membrane protein,
optionally, wherein the cell membrane protein is a cytoskeleton protein, a cluster of differentiation (CD) protein or beta actin.

11. The method of any preceding claim, wherein the second aptamer further comprises a detectable moiety binding domain responsive to the analyte binding to the domain that binds the analyte.

12. The method of claim 11, wherein the partition further comprises a detectable moiety,
wherein the detectable moiety binding domain uptakes the detectable moiety in response to the analyte binding to the domain.

13. The method of claim 12, further comprising detecting uptake of the detectable moiety by the detectable moiety binding domain.

14. The method of claim 13, wherein the detectable moiety binding domain is bound to the detectable moiety and releases the detectable moiety in response to the analyte binding to the domain that binds the analyte.

15. The method of claim 14, further comprising: detecting release of the detectable moiety by the detectable moiety binding domain.

16. A method of identifying or characterizing a cell as secreting an analyte comprising:
(a) providing a partition comprising,
a cell,
a plurality of nucleic acid barcode molecules comprising a partition-specific barcode sequence; and
a reporter agent for detecting the analyte comprising: a first aptamer comprising:
a first domain that binds the analyte;
a second domain comprising a reporter oligonucleotide; and
a third domain which is responsive to analyte binding by the first domain, and
(b) generating a barcoded nucleic acid molecule comprising a sequence of the reporter oligonucleotide or a reverse complement thereof and the partition-specific barcode sequence or reverse complement thereof.

17. The method of claim 16, wherein the partition further comprises a detectable moiety, and wherein the third domain comprises a subdomain capable of uptake of the detectable moiety in response to the analyte binding to the first domain.

18. The method of claim 17, further comprising: detecting uptake of the detectable moiety by the subdomain.

19. The method of claim 16, wherein the third domain further comprises a subdomain coupled to a detectable moiety, and
wherein the detectable moiety is released by the subdomain in response to the analyte binding to the first domain.

20. The method of claim 19, further comprising: detecting release of the detectable moiety by the subdomain.

21. The method of any of claims 16-20, wherein the reporter oligonucleotide comprises a reporter barcode sequence specific to the first aptamer and a capture handle sequence.

22. The method of claim 21, wherein the capture handle sequence is configured to couple to a capture sequence comprised in a first of the plurality of nucleic acid barcode molecules.

23. The method of claim 22, wherein the capture handle sequence is configured to couple to the capture sequence by complementary base pairing.

24. The method of claim 23, further comprising determining a sequence of the barcoded nucleic acid molecule.

25. The method of claim 24, further comprising determining secretion of the analyte based on the determined sequence of the barcoded nucleic acid molecule.

26. The method of claim 25, wherein the cell is identified or characterized as secreting the analyte based on the determining secretion of the analyte.

27. A method of identifying or characterizing a cell as secreting an analyte comprising:
(a) providing a partition comprising,
a cell;
a detectable moiety; and
a reporter agent for detecting the analyte comprising a first aptamer comprising:
a first domain that binds the analyte; and
a second domain responsive to the analyte binding the first domain, wherein the second domain comprises a subdomain capable of uptake of the detectable moiety in response to the analyte binding the first domain, and
(b) detecting uptake of the detectable moiety by the subdomain.

28. The method of claim 27, wherein the cell is identified or characterized as secreting the analyte if uptake of the detectable moiety is detected.

29. A method of identifying or characterizing a cell as secreting an analyte comprising:
(a) providing a partition comprising,
a cell;
a detectable moiety; and
a reporter agent for detecting the analyte comprising a first aptamer comprising:
a first domain that binds the analyte; and
a second domain responsive to the analyte binding the first domain, wherein the second domain comprises a subdomain capable of release of the detectable moiety in response to the analyte binding the first domain, and
(b) detecting release of the detectable moiety by the subdomain.

30. The method of claim 29, wherein the cell is identified or characterized as secreting the analyte if release of the detectable moiety is detected.

31. The method of any of claims 16-30, wherein the first aptamer further comprises a lipophilic moiety that embeds the first aptamer in the cell membrane.

32. The method of any of claims 16-31, wherein the first aptamer further comprises a cell protein binding domain,
optionally, wherein the cell protein binding domain binds a cell membrane protein,
optionally, wherein the cell membrane protein is a cytoskeleton protein, a cluster of differentiation (CD) protein or beta-actin.

33. The method of any of claims 16-30, wherein the first aptamer further comprises a first linking sequence, and
wherein the reporter agent further comprises a second aptamer comprising: a second reporter oligonucleotide, a second linking sequence and a lipophilic moiety that embeds the second aptamer in the cell membrane.

34. The method of claim 33, wherein the second aptamer binds a cell surface protein,
optionally, wherein the cell surface protein is a cytoskeleton protein, a CD protein or beta actin.

35. The method of claim 34, wherein the first linking sequence is configured to couple to the second linking sequence, and
wherein the partition is subject to conditions to link the first and the second aptamer via the first linking sequence and the second linking sequence.

36. The method of claim 35, wherein the first linking sequence and the second linking sequence are configured to couple by complementary base pairing.

37. The method of any of claims 33-36, wherein the second reporter oligonucleotide comprises a second reporter barcode sequence and a capture handle sequence.

38. The method of claim 37 as it depends from any of claims 16-25, further comprising generating a second barcoded nucleic acid molecule in the partition, wherein the second barcoded nucleic acid molecule comprises a sequence of the second reporter oligonucleotide or reverse complement thereof and the partition-specific barcode sequence or reverse complement thereof, or
as it depends from any of claims 27-30, wherein the partition further comprises a plurality of nucleic acid barcode molecules comprising a partition-specific barcode sequence, and wherein the method further comprises generating a second barcoded nucleic acid molecule in the partition, wherein the second barcoded nucleic acid molecule comprises a sequence of the second reporter oligonucleotide or reverse complement thereof and the partition-specific barcode sequence or reverse complement thereof.

39. The method of claim 38, further comprising identifying the second aptamer as being embedded in the cell membrane by the generating the second barcoded nucleic acid molecule.

40. The method of any preceding claim, further comprising identifying or characterizing the cell as secreting a second analyte.

41. The method of any of any preceding claim, further comprising, prior to (a):
culturing the cell under conditions to stimulate secretion of the analyte.

42. The method of claim 41, wherein the conditions comprise culturing the cell in presence of:
(a) a cytokine, wherein the cytokine is not the analyte; or
(b) a feeder cell; or
(c) a small molecule drug,
optionally, wherein the small molecule drug is conjugated to an antigen that mediates transport of the small molecule drug into the cell,
optionally, wherein the antigen mediates transport into the cell via a flipase-mediated pathway or a phosphatase.

43. The method of any of any of claims 1-40, wherein the provided partition further comprises an agent to stimulate the cell to secrete the analyte.

44. The method of claim 43, wherein the agent is:
(a) a cytokine, wherein the cytokine is not the analyte; or
(b) a feeder cell; or
(c) a small molecule drug,
optionally, wherein the small molecule drug is conjugated to an antigen that mediates transport of the small molecule drug into the cell,
optionally, wherein the antigen mediates transport via a flipase-mediated pathway or a phosphatase.

45. The method of any of claim 26, 28, 30 or 40 wherein the cell identified or characterized as secreting the analyte is further characterized or identified as a cell of interest.

46. The method of claim 45, wherein the cell of interest is a B regulatory cell and the analyte comprises one or more of IL-10, CCL-3 or CCL-4.

47. The method of claim 45, wherein the cell of interest is a tumor cell and the analyte comprises a growth factor,
optionally, wherein the growth factor comprises IGF, PDGF, EGF or PDGF.

48. The method of any of claims 45-47, further comprising enriching for the cell of interest.

49. The method of any of claims 45-47, further comprising selecting the cell of interest for further analysis,
optionally, wherein the further analysis comprises a generating an expression profile for the selected cell of interest,
optionally, wherein the expression profile is a transcription profile.

50. The method of any of claim 11-15, 17-20, or 27-30, wherein the detectable moiety produces a detectable signal.

51. The method of any of claims, 12, 13, 17, 18, 27, or 28, wherein the detectable moiety produces a detectable signal and the detectable signal is amplified upon the uptake.

52. The method of any of claim 14, 15, 19, 20, 29 or 30, wherein the detectable moiety produces a detectable signal and the detectable signal is amplified upon the release.

53. The method of any preceding claim, wherein the partition is a droplet, optionally, wherein the partition is a well or a flow cell.

54. The method of any preceding claim, wherein the detectable moiety is a fluorescent dye or colorimetric dye.

55. The method of any of claims 1-44, wherein the cell is an immune cell, or a tumor cell, or is of a sample from a subject,
optionally, wherein the sample from the subject is a blood or biopsy sample.

56. The method of any of claims 1-44, wherein the cell is partitioned from a reaction mixture comprising a plurality of cells.

57. The method of any of claims 1-44, wherein the analyte comprises:
(a) a nucleic acid,
optionally, wherein the nucleic acid comprises a single-stranded nucleic acid or double-stranded nucleic acid, optionally, wherein the nucleic acid comprises DNA or RNA,
optionally, wherein the nucleic acid comprises the RNA and the RNA is messenger RNA (mRNA); or
(b) a protein; or
(c) a cytokine,
optionally, wherein the cytokine comprises TNFα, CD27, CD30, CD40, IFN-α, IFN-β, IFN-γ, IFN-α, IFN-γ, IL-1, IL-2, IL-4, IL-6, IL-10, IL-13, IL-17RA, IL-17RB, IL-17RC, IL17RD, IL-17RE, IL-22, M-CSF, GM-CSF, or a chemokine; or
(d) a growth factor,
optionally, wherein the growth factor is one of IGF, EGF, PDGF, FGF; or
(e) an antibody or antigen-binding fragment thereof.

58. The method of claim 1 for characterizing a secreted antibody, or antigen-binding fragment thereof,
wherein the cell is an antibody-secreting cell and the analyte is an antibody or antigen-binding fragment thereof,
wherein the partition further comprises a second reporter agent for detecting binding of the antibody or antigen-binding fragment thereof to a target antigen,
wherein the second reporter agent comprises the target antigen coupled to a third reporter oligonucleotide,
wherein the third reporter oligonucleotide comprises a third barcode sequence specific to the target antigen and a third capture handle sequence configured to couple to the capture sequence comprised in the first of the plurality of nucleic acid barcode molecules,
wherein the conditions, at (b), are further sufficient to: (iv) bind the second reporter agent, via the target antigen, to the antibody or antigen-binding fragment thereof, and
wherein the method further comprises:
(c) generating a first, a second and/or a third barcoded nucleic acid molecule:
wherein the first barcoded nucleic acid molecule comprises a sequence of the first reporter oligonucleotide or reverse complement thereof and the partition-specific barcode sequence or reverse complement thereof,
wherein the second barcoded nucleic acid molecule comprises a sequence of the second reporter oligonucleotide or reverse complement thereof and the partition-specific barcode sequence or reverse complement thereof, and
wherein the third barcoded nucleic acid molecule comprises a sequence of the third reporter oligonucleotide or reverse complement thereof and the partition-specific barcode sequence or reverse complement thereof.

59. The method of claim 58, wherein the first and the second linking sequences comprise complementary nucleic acid sequences.

60. The method of claim 58 or 59, wherein the first, second and third capture handle sequences couple to the capture sequence by complementary base pairing.

61. The method of any of claims 58-60 further comprising determining sequence of the second barcoded nucleic acid molecule.

62. The method of claim 61, wherein the determined sequence of the second barcoded nucleic acid molecule characterizes the antibody-secreting cell as secreting the antibody or antigen-binding fragment thereof.

63. The method of any of claims 58-62, further comprising determining sequence of the third barcoded nucleic acid molecule.

64. The method of claim 63, wherein the determined sequence of the third barcoded nucleic acid molecule characterizes the antibody or antigen-binding fragment thereof as binding the target antigen.

65. The method of any of claims 58-64, further comprising determining sequence of the first barcoded nucleic acid molecule.

66. The method of claim 65, wherein the determined sequence of the first barcoded nucleic acid molecules identifies the first aptamer as being embedded in the cell membrane.

67. The method of any of claims 58-66, wherein the first aptamer further comprises a cell protein binding domain,
optionally, wherein the cell protein binding domain binds to a cell membrane protein,
optionally, wherein the membrane protein is a cytoskeleton protein, CD protein or beta-actin.

68. The method of any of claims 58-67, wherein the second aptamer further comprises a detectable moiety binding domain responsive to the antibody or antigen-binding fragment thereof binding to the domain that binds the antibody or antigen-binding fragment thereof.

69. The method of claim 68, wherein the partition further comprises a detectable moiety,
wherein the detectable moiety binding domain uptakes the detectable moiety in response to the antibody or antigen-binding fragment thereof binding to the domain.

70. The method of claim 69, further comprising detecting uptake of the detectable moiety by the detectable moiety binding domain.

71. The method of claim 68, wherein the detectable moiety binding domain is bound to the detectable moiety and releases the detectable moiety in response to the antibody or antigen-binding fragment thereof binding to the domain that binds the antibody or antigen-binding fragment thereof.

72. The method of claim 71, further comprising: detecting release of the detectable moiety by the detectable moiety binding domain.

73. The method of any of claims 16-26, for characterizing a secreted antibody, or antigen-binding fragment thereof,
wherein the cell is an antibody-secreting cell and the analyte is an antibody or antigen-binding fragment thereof,
wherein the partition further comprises a second reporter agent for detecting binding of the antibody or antigen-binding fragment thereof to a target antigen,
wherein the second reporter agent comprises the target antigen coupled to a second reporter oligonucleotide,
wherein the first aptamer of the reporter agent comprises a lipophilic moiety that embeds the first aptamer in the cell membrane,
wherein, in the provided partition: (i) the lipophilic moiety of the first aptamer embeds the first reporter agent in the membrane of the antibody-secreting cell, (ii) the first domain of the first aptamer of the reporter agent binds the antibody or antigen-binding fragment thereof, and (iii) the target antigen of the second reporter agent binds the antibody or antigen-binding fragment thereof; and
wherein, at (b), a second barcoded nucleic acid molecule is generated, wherein the second barcoded nucleic acid molecule comprises a sequence of the second reporter oligonucleotide or a reverse complement thereof and the partition-specific barcode sequence or reverse complement thereof.

74. The method of claim 73, wherein the second reporter oligonucleotide comprises a second reporter barcode sequence specific to the target antigen and a second capture handle sequence.

75. The method of claim 74, wherein the second capture handle sequence is configured to couple to a second capture sequence comprised in a second of the plurality of nucleic acid barcode molecules.

76. The method of claim 75, wherein the second capture handle sequence is configured to couple to the second capture sequence by complementary base pairing.

77. The method of any of claims 73-76, further comprising determining a sequence of the second barcoded nucleic acid molecule.

78. The method of claim 77, further comprising characterizing the secreted antibody or antigen-binding fragment thereof as binding the target antigen based on the sequence of the second barcoded nucleic acid molecule.

79. The method of any of claims 27-30, for characterizing a secreted antibody, or antigen-binding fragment thereof,
wherein the cell is an antibody-secreting cell and the analyte is an antibody or antigen-binding fragment thereof,
wherein the partition further comprises: (i) a plurality of nucleic acid barcode molecules comprising a partition-specific barcode sequence, and (ii) a second reporter agent for detecting binding of the antibody or antigen-binding fragment thereof to a target antigen,
wherein the second reporter agent comprises the target antigen coupled to a reporter oligonucleotide,
wherein the first aptamer of the reporter agent comprises a lipophilic moiety that embeds the first aptamer in the cell membrane,
wherein, in the provided partition: (i) the lipophilic moiety embeds the first aptamer of the reporter agent in the membrane of the antibody-secreting cell, (ii) the first domain of the first aptamer of the reporter agent binds the antibody or antigen-binding fragment thereof, and (iii) the target antigen of the second reporter agent binds the antibody or antigen-binding fragment thereof; and
wherein the method further comprises:
(c) generating a barcoded nucleic acid molecule comprising a sequence of the reporter oligonucleotide or a reverse complement thereof and the partition-specific barcode sequence or reverse complement thereof.

80. The method of claim 79, wherein the reporter oligonucleotide comprises a reporter barcode sequence specific to the target antigen and a capture handle sequence.

81. The method of claim 80, wherein the capture handle sequence is configured to couple to a capture sequence comprised in a first of the plurality of nucleic acid barcode molecules by complementary base pairing.

82. The method of any of claims 79-81 further comprising determining a sequence of the barcoded nucleic acid molecule.

83. The method of claim 82, further comprising characterizing the secreted antibody or antigen-binding fragment thereof as binding the target antigen based on the sequence of the barcoded nucleic acid molecule.

84. The method of any of claims 73-83, wherein the first aptamer further comprises a cell protein binding domain.
optionally, wherein cell protein binding domain binds a cell membrane protein,
optionally, wherein the cell membrane protein is a cytoskeleton protein, a cluster of differentiation protein or beta-actin.

85. The method of claim 33, as it depends from claims 16-26, for characterizing a secreted antibody, or antigen-binding fragment thereof,
wherein the cell is an antibody-secreting cell and the analyte is an antibody or antigen-binding fragment thereof,
wherein the partition further comprises a second reporter agent for detecting binding of the antibody or antigen-binding fragment thereof to a target antigen,
wherein the second reporter agent comprises the target antigen coupled to a third reporter oligonucleotide,
wherein, in the provided partition: (i) the first aptamer and the second aptamer link via the first and the second linking sequences; (ii) the lipophilic moiety embeds the second aptamer in the membrane of the antibody-secreting cell, (iii) the first domain of the first aptamer binds the antibody or antigen-binding fragment thereof, and (iv) the target antigen of the second reporter agent binds the antibody or antigen-binding fragment thereof; and
wherein, at (b), a second barcoded nucleic acid molecule is generated, wherein the second barcoded nucleic acid molecule comprises a sequence of the third reporter oligonucleotide or a reverse complement thereof and the partition-specific barcode sequence or reverse complement thereof.

86. The method of claim 85, wherein the third reporter oligonucleotide comprises a third reporter barcode sequence specific to the target antigen and a third capture handle sequence configured to couple to a second capture sequence comprised in a second of the plurality of nucleic acid barcode molecules.

87. The method of claim 86, wherein the third capture handle sequence is configured to couple to the second capture sequence by complementary base pairing.

88. The method of any of claims 85-87, further comprising determining a sequence of the second barcoded nucleic acid molecule.

89. The method of claim 88, further comprising characterizing the secreted antibody or antigen-binding fragment thereof as binding the target antigen based on the sequence of the second barcoded nucleic acid molecule.

90. The method of any of claims 85-89, wherein the second reporter oligonucleotide comprises a second reporter barcode sequence and a second capture handle sequence.

91. The method of claim 90 further comprising: generating a third barcoded nucleic acid molecule in the partition, wherein the third barcoded nucleic acid molecule comprises a sequence of the second reporter oligonucleotide or reverse complement thereof and the partition-specific barcode sequence or reverse complement thereof.

92. The method of claim 91, further comprising determining sequence of the third barcoded nucleic acid molecule.

93. The method of claim 92, further comprising determining the second aptamer as being embedded in the cell membrane based on the determining the sequence of the third barcoded nucleic acid molecule.

94. The method of claim 33, as it depends from claims 27-30, for characterizing a secreted antibody, or antigen-binding fragment thereof,
wherein the cell is an antibody-secreting cell and the analyte is an antibody or antigen-binding fragment thereof,
wherein the partition further comprises: (i) a plurality of nucleic acid barcode molecules comprising a partition-specific barcode sequence, and (ii) a second reporter agent for detecting binding of the antibody or antigen-binding fragment thereof to a target antigen,
wherein the second reporter agent comprises the target antigen coupled to a reporter oligonucleotide,
wherein the first aptamer of the reporter agent comprises a lipophilic moiety that embeds the first aptamer in the cell membrane,
wherein, in the provided partition: (i) the first aptamer and the second aptamer link via the first and the second linking sequences; (ii) the lipophilic moiety embeds the second aptamer in the membrane of the antibody-secreting cell, (iii) the first domain of the first aptamer binds the antibody or antigen-binding fragment thereof, and (iv) the target antigen of the second reporter agent binds the antibody or antigen-binding fragment thereof; and
wherein the method further comprises:
(c) generating a barcoded nucleic acid molecule comprising a sequence of the reporter oligonucleotide or a reverse complement thereof and the partition-specific barcode sequence or reverse complement thereof.

95. The method of claim 94, wherein the reporter oligonucleotide comprises a reporter barcode sequence specific to target antigen and a capture handle sequence.

96. The method of claim 95, wherein the capture handle sequence is configured to couple to the capture sequence comprised in a first nucleic acid barcode molecule of the plurality of nucleic acid barcode molecules by complementary base pairing.

97. The method of any of claims 94-96, further comprising determining a sequence of the barcoded nucleic acid molecule.

98. The method of claim 97, further comprising characterizing the secreted antibody or antigen-binding fragment thereof as binding the target antigen based on the sequence of the barcoded nucleic acid molecule.

99. The method of any of claims 94-98, wherein the second reporter oligonucleotide comprises a second reporter barcode sequence and a second capture handle sequence.

100. The method of any of claims 98-99, further comprising: generating a second barcoded nucleic acid molecule, wherein the second barcoded nucleic acid molecule comprises a sequence of the second reporter oligonucleotide or reverse complement thereof and the partition-specific barcode sequence or reverse complement thereof.

101. The method of claim 100, further comprising identifying the second aptamer as being embedded in the cell membrane by the generating the second barcoded nucleic acid molecule.

102. The method of any of claims 85-101, wherein the second aptamer further comprises a cell protein binding domain,
optionally, wherein the cell protein binding domain binds a cell membrane protein,
optionally, wherein the cell membrane protein is a cytoskeleton protein, a cluster of differentiation protein or beta-actin.

103. The method of any of claims 85-102, wherein the first linking sequence and the second linking sequence are configured to couple by complementary base pairing.

104. The method of any of claims 58-103, further comprising, prior to (a):
culturing the antibody-secreting cell under conditions to stimulate secretion of the antibody or antigen-binding fragment thereof.

105. The method of any of claims 58-103, wherein the provided partition further comprises an agent to stimulate the antibody-secreting cell to secrete the antibody or antigen-binding fragment thereof.

106. The method of any of claims 58-105, further comprising generating an additional barcoded nucleic acid molecule,
wherein the additional barcoded nucleic acid molecule comprises a nucleic acid sequence encoding the antibody or antigen-binding fragment thereof or reverse complement thereof and the partition-specific barcode sequence or reverse complement thereof.

107. The method of claim 106, wherein an additional nucleic acid barcode molecule of the plurality of nucleic acid barcode molecules comprises an additional capture sequence configured to couple to an mRNA or DNA analyte encoding the antibody or antigen-binding fragment thereof.

108. The method of claim 107, wherein the additional capture sequence comprises a polyT sequence.

109. The method of claim 107, wherein the additional capture sequence is configured to couple to non-templated nucleotides appended to a cDNA reverse transcribed from the mRNA analyte.

110. The method of claim 109, wherein the non-templated nucleotides appended to the cDNA comprise a cytosine.

111. The method of claim 110, wherein the additional capture sequence configured to couple to the cDNA comprise a guanine.

112. The method of claim 111, wherein coupling of the additional capture sequence to the non-templated cytosine extends reverse transcription of the cDNA into the additional nucleic acid barcode molecule.

113. The method of any of claims 106-112, further comprising determining sequence of the additional barcoded nucleic acid molecule.

114. The method of claim 113, further comprising identifying the antibody or antigen binding fragment thereof based on the determined sequence of the additional barcoded nucleic acid molecule.

115. A partition comprising:
a cell; and
a reporter agent comprising a first aptamer comprising:
a first domain that binds an analyte; and
a second domain responsive to the analyte binding the first domain.

116. A kit comprising, instructions for use thereof and:
a reporter agent comprising a first aptamer comprising:
a first domain that binds an analyte; and
a second domain responsive to the analyte binding the first domain,
wherein the kit is for: detecting secretion of the analyte by a cell, or characterizing an analyte secretion profile of a cell, or identifying a cell as a cell of interest, or enriching for a cell as a cell of interest.

117. A method of identifying or characterizing a cell as secreting an analyte comprising:
(a) partitioning a reaction mixture, or a portion thereof, into a plurality of partitions, wherein the reaction mixture comprises:
a plurality of cells, and
a first reporter agent, wherein the first reporter agent comprises a first and a second aptamer,
wherein the first aptamer comprises a first reporter oligonucleotide, a first linking sequence, and a lipophilic moiety that embeds the first aptamer in the cell membrane,
wherein the second aptamer comprises a second reporter oligonucleotide, a second linking sequence, and a domain that binds the analyte;
wherein the first reporter oligonucleotide comprises a first reporter barcode sequence specific to the first aptamer and a first capture handle sequence,
wherein the second reporter oligonucleotide comprises a second reporter barcode sequence specific to the second aptamer and a second capture handle sequence,
wherein the first and the second linking sequences link the first and the second aptamers;
wherein a cell of the plurality of cells is coupled to the first reporter agent, said first reporter agent bound to the analyte, as a result of:
(i) the lipophilic moiety of the first aptamer embedding in the cell membrane, and
(ii) the secreted analyte binding to the domain that binds the analyte of the second aptamer;
wherein the partitioning provides a partition, comprising:
(i) the cell coupled to the first reporter agent, said first reporter agent bound to the analyte, and
(ii) a plurality of nucleic acid barcode molecules comprising a partition-specific barcode sequence; and
(b) generating barcoded nucleic acid molecules, wherein the barcoded nucleic acid molecules comprise a sequence of: (i) the first reporter oligonucleotide or a reverse complement thereof and the partition-specific barcode sequence or a reverse complement thereof, and/or
(ii) the second reporter oligonucleotide or a reverse complement thereof and the partition-specific barcode sequence or a reverse complement thereof.

118. The method of claim 117, wherein the first aptamer further comprises a cell protein-binding domain,
optionally, wherein the cell protein binding domain binds to a cell membrane protein,
optionally, wherein the cell membrane protein is a cytoskeleton protein, a cluster of differentiation (CD) protein or beta actin.

119. The method of claim 117 or 118, wherein the second aptamer further comprises a detectable moiety binding domain responsive to the analyte binding to the domain that binds the analyte.

120. The method of claim 119, wherein the partition further comprises a detectable moiety,
wherein the detectable moiety binding domain uptakes the detectable moiety in response to the analyte binding to the domain.

121. The method of claim 120, further comprising detecting uptake of the detectable moiety by the detectable moiety binding domain.

122. The method of claim 119, wherein the the detectable moiety binding domain is bound to the detectable moiety and releases the detectable moiety in response to the analyte binding to the domain that binds the analyte.

123. The method of claim 122, further comprising: detecting release of the detectable moiety by the detectable moiety binding domain.

124. The method of any of claims 117-123, further comprising determining sequence of the second barcoded nucleic acid molecule.

125. The method of claim 124, further comprising determining secretion of the analyte based on the determined sequence of the barcoded nucleic acid molecule.

126. The method of any of claims 117-125, for characterizing a secreted antibody, or antigen-binding fragment thereof,
wherein the cell is an antibody-secreting cell and the analyte is an antibody or antigen-binding fragment thereof,
wherein the partition further comprises a second reporter agent for detecting binding of the antibody or antigen-binding fragment thereof to a target antigen,
wherein the second reporter agent comprises the target antigen coupled to a third reporter oligonucleotide,
wherein the third reporter oligonucleotide comprises a third barcode sequence specific to the target antigen and a third capture handle sequence,
wherein the cell of the plurality of cells is coupled to the first reporter agent, said first reporter agent bound to the antibody or antigen-binding fragment thereof, said antibody or antigen-binding fragment thereof bound to the target antigen,
wherein, at step (b), the method further comprises generating a first further and/or second further barcoded nucleic acid molecule, wherein:
the first further barcoded nucleic acid molecule comprises a sequence of the third reporter oligonucleotide or reverse complement thereof and the partition-specific barcode sequence of a reverse complement thereof, and
the second further barcoded nucleic acid molecule comprises a sequence encoding the antibody or antigen-binding fragment thereof, or a reverse complement thereof, and the partition-specific barcode sequence or a reverse complement thereof.
